(19) United States
(12) Patent Application Publication
(10) Pub. No.: US 2026/0346039 A1
(43) Pub. Date: Nov. 5, 2026

(54) MAGNETIC PULSE THERAPY DEVICE WITH SOLENOID DISTAL-END FLUX ENHANCEMENT FOR PAIN TREATMENT

(71) Applicant: Innovator Corporation, Browns Point, WA (US)

(72) Inventor: David Kovanen, Browns Point, WA (US)

(73) Assignee: Innovator Corporation, Browns Point, WA (US)

(21) Appl. No.: 19/743,443

(22) Filed: Jul. 15, 2026

Related U.S. Application Data
(63) Continuation of application No. 19/560,084, filed on Mar. 8, 2026.
Application No. 19/560,084 is a continuation of application No. 30/011,294, filed on Jul. 2, 2025, now Pat. No. D1121169.
Application No. 30/011,294 is a continuation of application No. 29/959,319, filed on Aug. 24, 2024, now Pat. No. D1088255.
Application No. 19/560,084 is a continuation of application No. 30/011,285, filed on Jul. 2, 2025.
Application No. 30/011,285 is a continuation of application No. 29/959,319, filed on Aug. 24, 2024, now Pat. No. D1088255.
Application No. 19/560,084 is a continuation of application No. 19/096,292, filed on Mar. 31, 2025, now Pat. No. 12,599,777.
Application No. 19/096,292 is a continuation of application No. 19/072,219, filed on Mar. 6, 2025, now Pat. No. 12,415,087.
Application No. 19/072,219 is a continuation of application No. 18/905,994, filed on Oct. 3, 2024, now Pat. No. 12,377,281.
Application No. 18/905,994 is a continuation of application No. 18/432,044, filed on Feb. 4, 2024, now Pat. No. 12,268,891.
(63) Continuation of application No. 30/047,013, filed on Feb. 6, 2026.
Application No. 30/047,013 is a continuation of application No. 30/011,294, filed on Jul. 2, 2025, now Pat. No. D1121169.
Application No. 30/011,294 is a continuation of application No. 30/011,285, filed on Jul. 2, 2025.
(63) Continuation of application No. 30/047,011, filed on Feb. 6, 2026.
Application No. 30/047,011 is a continuation of application No. 30/011,294, filed on Jul. 2, 2025, now Pat. No. D1121169.
Application No. 30/011,294 is a continuation of application No. 30/011,285, filed on Jul. 2, 2025.
(60) Provisional application No. 63/844,359, filed on Jul. 15, 2025.
(60) Provisional application No. 63/844,338, filed on Jul. 15, 2025.
(60) Provisional application No. 63/923,939, filed on Nov. 24, 2025.
(60) Provisional application No. 63/923,922, filed on Nov. 24, 2025.
(60) Provisional application No. 63/925,789, filed on Nov. 26, 2025.
(60) Provisional application No. 63/935,087, filed on Dec. 9, 2025.
(60) Provisional application No. 63/964,132, filed on Jan. 20, 2026.
(60) Provisional application No. 63/972,436, filed on Jan. 30, 2026.

(30) Foreign Application Priority Data
Aug. 14, 2025 (CN) 202511137736.8

(51) Int. Cl. A61N 2/02; A61N 2/00

(52) U.S. Cl. CPC A61N 2/02; A61N 2/008

(57) ABSTRACT

A Magnetic Pulse Therapy Device (MPTD) includes a solenoidal applicator having a hollow treatment chamber configured to receive a foot, forefoot, hand, or other appendage. A segmented solenoid produces therapeutic magnetic pulses through the chamber. Distal-end flux-management structures improve Magnetic Flux Density and field organization near distal anatomical tissue, including distal digital and forefoot regions. The structures may include chamber-geometry-based solenoid elongation, increased distal longitudinal turn density, commingled Windings of separately energizable coil segments, axial flux guides, Flux-Guide Coils, a distal flux lens, a distal flux target, and integrated guide-target yokes. The lens reduces sidewall divergence, the target draws Core Flux through the distal opening, and the guides provide a lower-reluctance return path. Temperature-responsive optimization uses temperature sensing, thermal-budget determination, model data, and pulse-parameter control to increase or preserve delivered magnetic-flux dosage while maintaining acceptable temperature.

### CROSS-REFERENCE TO RELATED APPLICATIONS

| Application | | |
|---|---|---|
| No. | Date Filed | Title |
| Current | Herewith | MAGNETIC PULSE THERAPY DEVICE WITH SOLENOID |
| application | | DISTAL-END FLUX ENHANCEMENT FOR PAIN |
| | | TREATMENT |

is a continuation of:

| | | |
|---|---|---|
| 19/560,084 | Mar. 8, 2026 | MAGNETIC PULSE THERAPY APPLICATOR WITH |
| | | ANATOMICALLY-SHAPED FOOT TREATMENT |
| | | CHAMBER |

which additionally claims the benefit of provisional application(s):

| | | |
|---|---|---|
| 63/972,436 | Jan. 30, 2026 | DOSE-CONTROLLED PULSED ELECTROMAGNETIC |
| | | THERAPY SYSTEM USING PREDICTIVE THERMAL |
| | | MODELING AND ADAPTIVE PULSE SCHEDULING |
| 63/964,132 | Jan. 20, 2026 | ADVANCED MAGNETIC PULSE THERAPY DOSING |
| | | METHODOLOGY & SYSTEM |
| 63/935,087 | Dec. 9, 2025 | MAGNETIC PULSE THERAPY DEVICE AND METHODS |
| | | WITH PRE-HEATING FOR ENHANCED |
| | | NEUROMODULATORY EFFICACY |
| 63/925,789 | Nov. 26, 2025 | MAGNETIC PULSE THERAPY (MPTD) DEVICE WITH |
| | | NERVE CONE STEERING |
| 63/923,939 | Nov. 24, 2025 | MAGNETIC PULSE THERAPY (MPTD) DEVICE WITH |
| | | NERVE CONE STEERING |
| 63/923,922 | Nov. 24, 2025 | MAGNETIC PULSE THERAPY DEVICE WITH |
| | | TRANSVERSE SEGMENTED SOLENOID |
| 63/844,359 | Jul. 15, 2025 | MAGNETIC PULSE THERAPY DEVICE (MPTD) |
| | | SOLENOID DISTAL END FLUX ENHANCEMENTS |
| 63/844,338 | Jul. 15, 2025 | PLANAR PLATE APPLICATOR FOR MAGNETIC PULSE |
| | | THERAPY |

and is also a continuation-in-part of:

| | | |
|---|---|---|
| 19/096,292 | Mar. 31, 2025 | MAGNETIC PULSE THERAPY DEVICE (MPTD) HIGH- |
| Patent | Issue Date: | POWER APPLICATOR FOR FOOT |
| 12,599,777 | Apr. 14, 2026 | |

which is a continuation-in-part of:

| | | |
|---|---|---|
| 19/072,219 | Mar. 6, 2025 | MAGNETIC PULSE THERAPY DEVICE (MPTD) WITH |
| Patent | Issue Date: | DYNAMIC DIRECTIONAL FLUX CONTROL |
| 12,415,087 | Sep. 16, 2025 | |

which is a continuation-in-part of:

| | | |
|---|---|---|
| 18/905,994 | Oct. 3, 2024 | MAGNETIC PULSE THERAPY DEVICE (MPTD) FOR THE |
| Patent | Issue Date: | TREATMENT OF PAIN |
| 12,377,281 | Aug. 5, 2025 | |

which is a continuation of:

| | | |
|---|---|---|
| 18/432,044 | Feb. 4, 2024 | MAGNETIC PULSE THERAPY DEVICE (MPTD) FOR THE |
| Patent | Issue Date: | TREATMENT OF PAIN |
| 12,268,891 | Apr. 8, 2025 | |

the entire specification of each of which is incorporated herein by reference.

### TECHNICAL FIELD

This disclosure relates to medical devices for magnetic pulse therapy, and more particularly to Magnetic Pulse Therapy Devices (“MPTDs”) configured to treat pain or other symptoms by applying time-varying magnetic fields to human or animal tissue.

More particularly, this disclosure concerns an MPTD applicator having a solenoidal treatment chamber configured to receive a foot, forefoot, hand, or other appendage. Preferred embodiments are configured for treating foot pain, including pain in the distal digital and forefoot regions associated with peripheral neuropathy, diabetic peripheral neuropathy, chemotherapy-induced peripheral neuropathy, plantar fasciitis, or related lower-extremity conditions.

The solenoid, flux guides, Flux-Guide Coils, flux lens, flux target, guide-target member, and pulse-control features are disclosed as structures of a medical treatment applicator. Their purpose is to improve delivery of treatment-relevant magnetic pulses to distal anatomical tissue positioned near the end of an MPTD treatment chamber.

### BACKGROUND AND ENGINEERING PROBLEM

A Magnetic Pulse Therapy Device treats tissue by producing magnetic pulses that pass through the treatment anatomy. The relevant engineering variables include Magnetic Flux Density B, rate of change dB/dt, pulse duration, pulse repetition rate, cumulative magnetic dose, treatment-volume size, placement repeatability, and temperature rise. In a foot-treatment MPTD, those variables must be delivered to a three-dimensional anatomical volume rather than merely to a skin-facing surface.

The foot is an especially demanding treatment target. Pain associated with peripheral neuropathy commonly begins in, or is most severe in, the distal digital region and forefoot. Target tissues can include distal phalanges, digital nerves, plantar digital branches, dorsal sensory branches, metatarsal regions, vascular structures, the plantar surface, the dorsum, and deeper internal tissue. A foot applicator that under-treats the distal digital region may miss the region where treatment is most needed.

A solenoid is attractive for foot treatment because it places the appendage inside the region where the magnetic field is most organized. In a puck, loop, mat, or surface coil, the treatment anatomy is beside a field source and is treated largely by non-core or fringe flux. That field can be useful near the surface, but it generally decays rapidly with distance and is sensitive to spacing, angle, and placement. A solenoidal treatment chamber changes the geometry: the foot is placed within the core of the magnetic source.

The related MPTD family disclosed a segmented solenoid formed from a plurality of axially aligned Coils. When selected Coils are concurrently energized with coordinated magnetic polarity, their fields add within the chamber and form an Operative Solenoid. That architecture allows a low-voltage, home-suitable MPTD to produce treatment-relevant magnetic pulses across a useful foot volume without the high voltages, massive conductors, heavy cables, and thermal burden of clinical high-power systems.

The segmented solenoid solves one large problem but exposes another. A long solenoid has a comparatively uniform field near its central length, but every finite solenoid has end effects. Near an axial end, flux begins to diverge. A portion of the field that would otherwise travel longitudinally through the core turns outward through the sidewall region and returns through air. In a classroom solenoid this is merely expected end behavior. In an MPTD foot applicator, it occurs at the distal digital end of the treatment chamber, where peripheral neuropathy symptoms are often most important.

The distal-end problem therefore has two parts. First, the magnitude of useful axial Magnetic Flux Density falls near the distal opening because part of the field has already begun to leave the core. Second, the field becomes less directionally organized. A distal digital region that should receive strong longitudinal Core Flux instead receives a weaker mixture of longitudinal, radial, and fringing components. The user may not perceive this immediately because magnetic pulse therapy does not produce an instantaneous visible effect, but the deficiency is directly measurable.

The disclosed invention treats distal-end flux loss as a medical-device magnetic-circuit problem. The object is not simply to make a solenoid stronger in the abstract. The object is to preserve or increase treatment-relevant Magnetic Flux Density and field organization near distal anatomical tissue while maintaining a practical treatment chamber, low-voltage pulse architecture, acceptable thermal behavior, and repeatable foot positioning.

The term “distal” is used from the perspective of the inserted appendage. In a foot applicator, the distal end is the digital end of the chamber and the proximal end is the entry end. The disclosed structures are most easily understood in the foot context, because that is the primary commercial and therapeutic use, but the same principles can be used in a forefoot applicator, hand applicator, or other appendage-receiving MPTD applicator in which a treatment target lies near an axial end of a solenoid.

### DESCRIPTION OF RELATED ART AND DIFFERENCES FROM THE PRESENT INVENTION

Most MPTD applicators control flux primarily through Coil shape, Coil placement, and input power. A puck applicator uses a wound conductor inside a hand-held housing. A loop applicator places a loop around or near a body part. A mat or pad applicator distributes multiple coils across a surface. These devices can be useful in some settings, but they normally apply magnetic field from outside the treatment target and rely on non-core flux. They do not place the foot inside a longitudinal solenoidal core and therefore do not face, recognize, or solve the same distal-end core-flux problem addressed by this application.

High-power clinical systems often compensate for inefficient field geometry by using hundreds or thousands of volts and very high instantaneous currents. This approach can produce large peak Magnetic Flux Density, but it also creates heat, weight, cable, safety, cost, and clinical-supervision problems. The present invention proceeds in the opposite direction. It seeks to make better use of the flux generated by a solenoidal MPTD applicator, so that useful distal-end flux is improved without relying on brute-force electrical power.

U.S. Pat. No. 5,518,495 to Kolt and U.S. Pat. No. 5,842,966 to Markoll disclose cylindrical or bobbin-like solenoidal treatment structures. These references are relevant because they show that an appendage can be placed in or near a solenoidal field. They do not disclose a segmented solenoid designed to produce treatment-relevant Core Flux throughout a foot chamber at low voltage. More importantly for the present disclosure, they do not disclose distal-end flux management using an axial flux guide, distal flux lens, distal flux target, or integrated guide-target member.

U.S. Pat. No. 9,550,067 to Fischell discloses a foot-related magnetic treatment applicator using very high electrical parameters. That reference is relevant because it recognizes the value of treating a foot with a surrounding magnetic structure. It differs from the present invention because it relies on high-power operation and does not teach the low-reluctance distal-end return structures described here. It does not teach a segmented-solenoid distal end in which a flux lens, flux target, and return yoke cooperate to preserve Core Flux at the distal digital end of the treatment chamber.

U.S. Pat. No. 9,849,302 to Fischell discloses a sandal-like arrangement using multiple coils near the foot. That approach is a surface or sandal-field approach, not a hollow treatment chamber with an axial core. It must address placement, heating, and flux cancellation problems associated with large nearby coils. It does not disclose the present magnetic circuit in which the treatment zone lies within a core and the distal-end structures are arranged to extend, collect, and return solenoidal flux.

U.S. Pat. No. 10,589,117 to Fischell discloses an integrated foot treatment device using a shelf-like applicator. It improves user handling compared with a hand-held clinical applicator, but it remains materially different from the present invention. The field is not managed as distal-end Core Flux in an appendage-receiving solenoid. The present invention uses the hollow treatment chamber itself as the treatment geometry and then improves the distal end of that solenoid.

U.S. Pat. No. 11,305,130 to Shukla illustrates reinforcement from multiple coils, including figure-eight style field interaction. That type of design can produce useful local field concentration, but it does not create a longitudinal appendage-receiving solenoid with a distal aperture. It therefore does not address the particular problem of solenoid end-effect fringing near the distal phalanges, nor does it teach using a distal flux target and axial return yoke to pull Core Flux through the end of a chamber.

U.S. Pat. Nos. 10,369,373 and 11,273,317 to Leung disclose mechanically reoriented coils that change field presentation by moving or rotating sources. Mechanical reorientation is a different solution space. The present invention uses a fixed solenoidal applicator and shapes flux with magnetic-circuit structures and pulse control. It is therefore compatible with a simple home-use form factor in which the user inserts a foot and does not manipulate or aim the applicator.

U.S. Patent Application Publication Nos. 2014/0249354 A1 and 2015/0328476 A1 to Anderson disclose high-permeability cores for concentrating magnetic flux. Iron-core devices exploit a familiar electromagnet principle: a ferromagnetic core conducts flux better than air. The present invention uses that principle selectively but does not simply insert an iron core into the treatment region. The chamber remains usable by the appendage, and high-permeability structures are placed around and beyond the treatment zone to shape the distal end without occupying the space where the foot belongs.

U.S. Patent Application Publication No. 2024/0278031 A1 to Babico et al., assigned to Regenesis Biomedical, Inc., discloses an applicator having emitter coils and a ferromagnetic field director. The field director is configured to direct magnetic field away from an unused side of the applicator and toward the patient. Babico is relevant because it uses high-permeability material to redirect magnetic field, but it addresses a planar or substrate-backed applicator problem rather than a finite-solenoid distal-end problem.

Babico differs from the present invention in both the field source and the field problem. Babico concerns emitter coils on or near a substrate and a backing field director that redirects field from an unused side. It does not disclose a foot-receiving segmented solenoid having a longitudinal hollow treatment chamber. It does not disclose axial Core Flux traveling through the chamber and then weakening at a distal digital opening. It also does not disclose a distal flux lens, distal flux target, axial flux guide, or L-shaped guide-target member configured to draw solenoidal Core Flux through the distal end and return external flux toward the proximal end.

### CN '815/'524 Diabetic-Foot Magnetic Therapy Reference

Chinese Patent Publication CN112494815A and corresponding Chinese Utility Model Patent CN216319524U disclose related subject matter directed to a diabetic-foot magnetic-therapy appliance. For convenience, the common disclosure of those Chinese documents is discussed here as the CN '815/'524 Reference.

The CN '815/'524 Reference deserves specific discussion because it is visually closer than a mat, puck, wand, or ordinary surface applicator. It discloses a foot-receiving cavity and multiple electromagnetic components positioned around selected regions of the foot. Visual similarity, however, is not magnetic equivalence. A foot cavity containing coils is not automatically a solenoidal MPTD treatment chamber, and the presence of material at a distal or proximal end is not automatically flux guidance.

The CN '815/'524 Reference discloses an outer housing, an inner housing, a first cavity inside the inner housing for receiving a foot, and a second cavity between the inner and outer housings for receiving electromagnetic components. It also discloses a control module, temperature sensors, and a current sensor. The electromagnetic components are divided into first coils at positions corresponding to the instep and forefoot, and second coils at positions corresponding to the distal digits and heel. The second coils have ferromagnetic cores.

In the disclosed embodiment, the first coil group includes three side-by-side coils identified as coils I 61, II 62, and III 63. The second coil group includes three coils at the bottom of the heel, a fourth second coil at an upper heel region, and a fifth second coil 75 at the distal digital region. The reference states that the first coils are coreless and produce weaker magnetic fields, while the second coils have iron cores and produce stronger magnetic fields. It further states that the first coils generate a magnetic field generally parallel to the foot surface, and that the second cored coils generate a magnetic field generally perpendicular to the foot surface.

That arrangement is best understood as a set of local perimeter field sources. The reference assigns different local electromagnets to different anatomical regions and gives those electromagnets different strengths and directions. It does not describe the foot as being placed inside a coherent air-core solenoid treatment volume. It does not disclose a longitudinal measurement path through the chamber. It does not map field strength along such a path. It does not describe the local distal digital-region, heel-region, and forefoot-region coils as longitudinal segments of a common Operative Solenoid.

The distal coil 75 in the CN '815/'524 Reference is especially different from the present distal flux lens or flux target. Coil 75 is an active cored electromagnet. It has its own winding, current direction, pole pattern, inductance, hysteresis behavior, and local field. A distal flux lens or flux target of the present disclosure is instead a passive high-permeability member positioned with respect to the solenoid axis, distal aperture, and return path so that existing solenoidal Core Flux is encouraged to pass through the distal treatment region before returning externally.

If a cored electromagnet like CN coil 75 were placed at the distal end of a solenoidal core-flux applicator without a deliberate field map, it would not inherently enhance distal-end Core Flux. Depending on its polarity, timing, core geometry, and saturation state, it could attract some local flux, oppose the axial field, create cancellation, increase radial side fringing, shunt flux into a local core, or act as a magnetic obstruction or flux plug. The CN reference does not teach measuring whether coil 75 increases longitudinal distal Magnetic Flux Density in the treatment chamber. It teaches that a cored local coil produces a stronger local field than an uncored local coil.

The proximal and heel-region cored coils in the CN '815/'524 Reference are likewise not axial flux guides or return yokes. A return yoke in the present disclosure is arranged to receive flux after that flux has served the treatment region and to conduct it along a lower-reluctance external path toward the proximal region. The CN heel coils and associated ferromagnetic cores are local electromagnets intended to produce local perpendicular magnetic fields at the heel. They are not disclosed as passive return paths, are not arranged as a distal-to-proximal flux guide, and are not shown as preserving longitudinal solenoidal Core Flux.

The CN '815/'524 Reference also follows a different electrical design path. It expressly discusses making a field stronger by increasing turn count, including a comparison between 100 turns and 400 turns, and by adding ferromagnetic cores. That statement is reasonable for a same-current or steady-field comparison, but it points away from the low-voltage pulsed MPTD architecture disclosed here. For comparable geometry, inductance increases approximately with the square of turn count. A 400-turn coil can therefore have about sixteen times the inductance of a 100-turn coil, slowing current rise under a fixed low-voltage drive. In a short magnetic pulse, slower current rise can reduce dB/dt even if steady-state ampere-turn capability is larger.

The CN '815/'524 Reference refers generally to dynamic magnetic fields, but it does not disclose magnetic pulses in the engineering sense used in this application. It does not identify pulse initiation, pulse width, pulse repetition rate, pulse count, pulse waveform, rise time, fall time, bipolar reversal, capacitor-supported pulse discharge, branch drivers, or same-test measurement of Magnetic Flux Density at a specified elapsed time. The presence of an MCU, temperature sensors, and a current sensor does not supply those missing pulse-control features; the MCU is disclosed for monitoring and cutoff, not for forming a controlled low-voltage high-current MPTD pulse.

The CN '815/'524 Reference therefore does not disclose the present distal-end flux enhancements. It does not disclose solenoid elongation to move the end-effect region beyond distal anatomical tissue. It does not disclose distal longitudinal turn-density shaping in a low-inductance segmented solenoid. It does not disclose a passive distal flux lens that reduces sidewall escape. It does not disclose a passive distal flux target that draws solenoidal Core Flux through the distal aperture. It does not disclose axial flux guides or an L-shaped guide-target member forming a return-yoke path for external flux.

The engineering distinction is not semantic. In the present invention, the distal-end structures are evaluated by what they do to treatment-region B(t), longitudinal distal Magnetic Flux Density, radial divergence, and thermal behavior under the same-test energization condition. A mere object, coil, core, plate, or magnetic source at the distal or proximal end of a foot cavity does not meet that function unless it participates in the solenoidal flux path in the disclosed way. The CN '815/'524 Reference places local field sources around a diabetic foot; the present invention improves distal delivery of therapeutic magnetic pulses in a solenoidal MPTD treatment chamber.

U.S. Patent Application Publication No. 2010/0185041 to Lee describes a circumferential magnetic field and related vector-potential concepts. It is not directed to the same problem. The present invention is not based on producing a circumferential therapeutic field around a cylinder. It is based on preserving and steering longitudinal Core Flux in a finite solenoid, especially where that flux would otherwise diverge at the distal end of the treatment chamber.

The distinction from the prior art can be summarized technically. Prior devices use stronger coils, different coil shapes, coil backing plates, iron cores, mechanical movement, or local field overlap. The present invention uses the existence of a solenoidal treatment chamber as the starting point and then modifies the magnetic circuit at the distal axial end. That solution is possible because the useful treatment flux is organized Core Flux inside the solenoid. A planar pad or puck does not have a comparable distal-core-flux end effect to correct.

### TERMINOLOGY AND MEASUREMENT CONVENTIONS

The following terminology is used to make the engineering disclosure readable and to harmonize this application with related applications in the MPTD family. The definitions are intended to be consistent with ordinary electrical and magnetic engineering usage unless the text provides a contextual definition for this family of devices.

An “MPTD” is a Magnetic Pulse Therapy Device. The device produces time-varying magnetic fields, or magnetic pulses, using electrically energized conductors. This application uses MPTD terminology throughout. References that use different industry terminology are discussed only to identify and distinguish the prior art.

Magnetic Flux Density is denoted by B and is measured in tesla, with millitesla abbreviated mT. In this application, a statement that an applicator produces a specified Magnetic Flux Density should be read with the relevant pulse timing, sensor position, and energization condition in mind, because a pulsed inductive load does not reach its final current or field instantaneously.

A “Coil” is an electrical unit that is energized and controlled as a unit and that includes one or more Windings. A Coil has electrical terminals or equivalent electrical connection points through which a driver circuit energizes the Coil. A Coil can contain a single Winding, multiple series Windings, multiple parallel Windings, or a physically distributed conductor pattern, provided the grouped conductor structure is energized as a unit.

A “Winding” is a wound portion of conductor that follows at least part of a path around a core, chamber, mandrel, or air space. The term is descriptive of the conductor geometry and does not by itself determine whether the conductor is separately controlled. A Winding can be a portion of a Coil, a complete Coil, or one of several conductor paths that collectively form a Coil.

A “Turn,” “wrap,” “loop,” or “wrapping” refers to one encircling traversal, or substantially one encircling traversal, of a conductor around a core, chamber, or other region. A Turn need not be a perfect geometric circle. In the illustrated applicator, the Turn follows the non-circular cross-sectional contour of the treatment chamber.

A “Solenoid” is a conductor arrangement having a purposeful axial length and producing a magnetic field through a core or chamber when energized. A solenoid can be formed by one continuous helical Winding or by a plurality of Coils arranged along a longitudinal axis. A solenoid used for treatment preferably has enough longitudinal extent to create a meaningful region of Core Flux rather than merely a local loop field.

A “Segmented Solenoid” is a solenoid formed by a plurality of Coils or Coil segments arranged so that their magnetic fields intentionally interact along a longitudinal extent. The Coils need not be identical. They can differ in size, number of Turns, spacing, wire gauge, winding direction, driver connection, or pulse timing, provided the selected Coils cooperate to form a useful solenoidal field in at least one operating mode.

“Commingled Windings” are winding portions of adjacent or nearby Coil segments that are both present at common longitudinal positions or otherwise physically intermingled along at least part of the solenoid length while the Coil segments remain electrically separate or separately energizable. Commingled Windings may be deliberately produced by spin winding, automated winding, semi-automated winding, hand-assisted winding, tapered-core winding, or another process that allows conductor portions to nest, fan, drift, or overlap across adjacent segment zones.

A “longitudinal overlap region” is a region along the proximal-distal direction of the solenoid in which winding portions of two separately energizable Coil segments occupy common longitudinal positions. The overlap region can occur along only one side of the solenoid, such as a side corresponding to an upper boundary of the treatment chamber, or can extend around a larger portion of the winding path. The term does not require a full Turn of one Coil segment to overlap a full Turn of another Coil segment.

An “Operative Solenoid” exists when a selected combination of Coils cooperates to produce Core Flux along a treatment-relevant longitudinal region. A practical test is the same-test energization comparison used in the related family: under corresponding measurement conditions, a selected concurrently energized Coil combination produces greater Magnetic Flux Density at corresponding points along a longitudinal measurement path than any one Coil of that selected combination produces alone at those same points. The relationship does not require a simple arithmetic sum; real fields are affected by geometry, end effects, sensor orientation, and coupling.

A “selected combination” means the group of Coils chosen for a particular pulse, measurement, or operating mode. The selected combination need not include every installed Coil. A device can use one selected combination to form an Operative Solenoid and other combinations for localized emphasis, thermal balancing, diagnosis, comfort adjustment, or Dynamic Directional Flux Control.

A “same-test energization condition” means that the selected combination and the individual Coils being compared are measured under corresponding conditions. The comparison should use the same nominal supply voltage or corresponding current condition, the same pulse initiation convention, the same commanded pulse duration or specified elapsed time, the same sensor position and orientation, the same fixture, and the same environmental condition. The purpose is to test magnetic cooperation rather than test artifacts.

A “longitudinal measurement path” is a path used to characterize treatment-region Magnetic Flux Density along the proximal-distal direction of the treatment chamber. In a foot applicator, the path can extend through a distal digit, forefoot, metatarsal, midfoot, plantar, dorsum, or chamber-center region. The path can be straight, curved, angled, piecewise straight, or defined by an array of corresponding measurement points. The important feature is that it has a defined longitudinal extent and repeatable sensor positions.

A “Reference Distal Point” or “RDP” is a treatment-chamber-geometry reference point used to determine whether a solenoid is elongated distally. The RDP is not determined by measuring a particular user's foot. In foot embodiments, the RDP may be selected from chamber-defined candidate points, such as a distal-stop-derived candidate point when present, height-derived candidate points, and, in some embodiments, proximal-stop-derived candidate points, as described in greater detail below with respect to FIGS. 26A-26C.

For distal-end placement measurements, the treatment chamber can have a distal chamber extent defined by the distal-most longitudinal position of the portion of the applicator that defines the user-receiving treatment chamber, measured along the distal insertion direction. If the chamber includes a closed distal wall or toe stop, the distal chamber extent may be the distal-most position of that wall or stop. If the chamber is open distally, the distal chamber extent may be defined by the distal aperture, distal rim, distal perimeter, or distal-most chamber-defining structure. This convention allows distal placement to be measured even when the treatment chamber does not have a closed distal end.

For distal ferromagnetic flux-enhancement structures, a distal reference position can be the more proximal of the distal chamber extent and the distal-most winding extent of the solenoid. A Flux Lens, Flux Target, axial Flux Guide, Guide-Target Member, or combination of those structures is considered a distal flux-enhancement structure when at least a portion of the structure, such as a distal portion, is positioned distally of a reference plane perpendicular to the distal insertion direction and located 15 mm proximal of that distal reference position. This definition captures structures located at or near the distal end of the chamber, including structures partly beyond the chamber and structures that extend longitudinally along an exterior side region of the solenoid.

The “distal digital region” of a foot includes the toes, distal phalanges, associated digital nerves, distal plantar and dorsal digital branches, and adjacent soft tissue. The term is used because the medical treatment target is anatomical tissue, not merely a geometric end of a product. Where this specification uses the word “toe” for readability, it refers to the distal digital or phalangeal region unless context requires otherwise.

A “treatment chamber” and a “treatment zone” are related but distinct structures and are given distinct reference numerals. The treatment chamber 225 is the physical space into which a foot, forefoot, hand, or other appendage is received; it is the appendage-receiving volume defined by the applicator core structure 220 and bounded by the chamber-defining structure. The treatment zone 120 is the active portion of the treatment chamber 225, namely the region within the chamber through which treatment-relevant Core Flux is produced when a selected combination of Coils 105 is energized. The treatment zone 120 is at least a portion of, and is frequently smaller than, the treatment chamber 225; that is, the treatment zone is a subset of the treatment chamber. Accordingly, the Coils 105 at least partially encircle the treatment chamber 225 and, when energized, form the treatment zone 120, such that the treatment zone 120 is at least a portion of the treatment chamber 225. This terminology is used consistently throughout this disclosure: the Coils encircle the chamber, and the Coils form the zone. The two terms are not interchangeable, and each is identified by its own reference numeral.

“Core Flux” is magnetic flux traveling through the area encircled by a Coil or by the selected Coils forming an Operative Solenoid. In the present applicator, the treatment chamber occupies the core, so the foot is placed directly into the region where Core Flux is produced. Core Flux is generally more spatially organized and more longitudinally aligned than non-core flux produced around the outer perimeter of a surface coil.

“Non-Core Flux” is magnetic flux outside the core region or field that leaves the sides, edges, or outer perimeter of a Coil arrangement before becoming useful treatment flux. In a planar pad or puck, much of the available treatment field is non-core flux. In a solenoidal applicator, some non-core flux is unavoidable because all flux returns through some external path, but the useful treatment objective is to maintain as much organized Core Flux as practical through the treatment zone.

“Coherence” in this application does not mean optical or radio-frequency phase coherence. It means the degree to which magnetic flux in the treatment zone remains directionally aligned, spatially organized, and resistant to premature radial divergence. A coherent distal field has a strong longitudinal component and relatively reduced radial sidewall escape. The term is used because it describes the engineering quality of the treatment field in the chamber.

A “Flux Guide” is a high-permeability structure placed to conduct or guide magnetic flux along a desired path. Accepted engineering names for such a structure include magnetic yoke, return yoke, field guide, flux return, flux conductor, magnetic return path, and keeper, depending on geometry and use. In this application, axial flux guides are elongated high-permeability members arranged along the outside of the solenoid to provide a lower-reluctance return path from the distal region toward the proximal region.

A “Flux-Guide Coil,” “guide-bias winding,” or “active guide winding” is an auxiliary Winding disposed on, around, adjacent to, or electromagnetically coupled to a Flux Guide, guide-target member, L-shaped member, U-shaped member, bar guide, plate guide, or other longitudinal high-permeability structure outside the treatment chamber. A Flux-Guide Coil can be energized independently of the treatment Coils to magnetically bias, energize, assist, reset, or otherwise control the magnetic state of the guide structure. The term includes a pre-bias winding, active return-yoke winding, keeper winding, auxiliary yoke coil, or equivalent conductor arrangement.

A “Flux Lens” is a high-permeability distal structure that shapes the field at or near the distal opening. It is not a lens in the optical sense. It is a magnetic field shaper, pole-piece structure, distal yoke, flux concentrator, or perimeter field guide arranged to reduce premature radial escape and bias flux toward the distal aperture. A flux lens can be a ring, frame, frustum, segmented ring, plate array, or other high-permeability structure that surrounds or partly surrounds the distal opening.

A “Flux Target” is a high-permeability structure positioned beyond or adjacent to the distal end of the treatment chamber to draw Core Flux toward and through the distal opening. Accepted alternative names include magnetic target, collector plate, pole piece, terminal yoke, keeper plate, end plate, and flux collector. In this application, the Flux Target is not placed to occupy the treatment chamber; it is placed to influence the distal field and receive flux after it exits or approaches the distal end.

A “Guide-Target Member” is an integrated high-permeability member that performs both guide and target functions. A U-shaped strip that travels along one side, wraps around the distal end, and returns along another side is one example. An L-shaped member having a base plate along the applicator and an upright distal end plate is another example. The same physical piece can be called a yoke, return yoke, combined target and guide, magnetic keeper, or integrated flux return, depending on the feature being discussed.

An exterior side region of the solenoid includes a lower or inferior exterior region, a plantar-facing exterior region, a medial exterior region, a lateral exterior region, a superior exterior region, or another exterior region outside the treatment chamber along which a Flux Guide or guide-target portion can extend. Thus, a side region is not limited to a left or right side; a lower or bottom return-yoke region can also be an exterior side region for purposes of an axial Flux Guide.

“Relative permeability” is the ability of a material to support magnetic flux relative to free space. Ferromagnetic materials such as low-carbon steel, iron, silicon steel, nickel-iron alloy, and ferrite have much higher relative permeability than air while they remain in a useful unsaturated region. Actual permeability depends strongly on material composition, heat treatment, stress, frequency, field strength, geometry, and saturation state.

“Reluctance” is the magnetic-circuit opposition to flux and is analogous in a limited way to resistance in an electric circuit. For a simple magnetic path, reluctance is proportional to path length and inversely proportional to permeability and cross-sectional area. Thus, increasing permeability or cross-section, or shortening the path, reduces reluctance. This analogy is useful for understanding the disclosed structures, although real magnetic fields are distributed rather than confined to ideal wires.

Magnetic Flux Density is preferably measured using an AlphaLab MagWave fast magnetic waveform sensor, including a MagWave B-Probe, connected to a digital oscilloscope or equivalent waveform recorder. This arrangement allows the engineer to record B(t), peak B, B at a specified elapsed time, rise behavior, decay behavior, and dB/dt for a pulse. Alternative measurement methods are acceptable when they have adequate bandwidth, sensitivity, range, calibration, probe geometry, and time resolution for the pulse being measured.

Thermal performance is preferably measured with a thermal imaging camera during prototype, validation, and design-verification testing. Where the applicator is equipped with embedded temperature sensors, distal-mounted LM75-type digital sensors or equivalent sensors are preferably thermally coupled to distal Coils, Windings, winding-retention structures, or adjacent thermally representative structures using thermal paste or another thermally conductive interface. Thermal images and embedded sensors are complementary: the camera reveals spatial hot spots, while embedded sensors support closed-loop control in the product.

### SUMMARY OF THE INVENTION

The invention enhances delivery of therapeutic magnetic pulses to distal anatomical tissue in a solenoid-based MPTD treatment chamber. In a preferred foot embodiment, the enhancement improves Magnetic Flux Density and field organization in the distal digital and forefoot regions, where pain associated with peripheral neuropathy is often clinically important. The invention is a medical-device flux-management architecture, not a generic solenoid accessory.

The enhancement system uses one or more of several cooperating structures and control methods: solenoid elongation beyond a treatment-chamber-geometry reference point, controlled commingling or longitudinal overlap of separately energizable coil-segment Windings, increased distal longitudinal turn density, axial flux guides, actively energized Flux-Guide Coils, a distal flux lens, a distal flux target, and integrated guide-target members. Temperature-responsive magnetic-flux optimization, including pulse-duration control, guide-coil timing control, polarity control, waveform control, segment-selection control, forced-return control, model-based thermal-budget control, and thermal feedback, further allows the applicator to use available thermal headroom for increased distal output when conditions permit.

Solenoid elongation moves the natural end-effect region distally relative to the treatment chamber. In preferred foot embodiments, elongation is determined by comparing a distal-most winding extent of the solenoid to a Reference Distal Point selected from treatment-chamber geometry rather than from the actual anatomy of a particular user. This makes the elongation criterion definite for an applicator that may receive feet of different sizes and that may be open distally.

Increased distal longitudinal turn density changes the local ampere-turn distribution. Instead of treating every axial location as identical, the distal region receives additional conductor density or shorter longitudinal pitch. This increases local magnetomotive force and helps maintain useful field strength where a finite solenoid would normally weaken. Because added Turns also increase inductance and heat, the geometry is selected to improve distal treatment-region behavior without destroying the pulse rise-time advantage of the segmented solenoid.

Controlled commingling of adjacent separately energizable coil segments is another flux-enhancement feature. Conventional segmented-solenoid construction often favors sharply separated segment coils, separate bobbins, stacked bobbins, or physical dividers. In the disclosed MPTD applicator, however, the magnetic flux acts directly on the appendage-receiving treatment chamber rather than on a movable armature that requires precise longitudinal positioning. Commingled or overlapping winding portions can therefore improve longitudinal field coherence, reduce inter-segment flux aberrations, smooth the axial ampere-turn distribution, and improve manufacturability while preserving separately energizable coil segments.

Axial flux guides provide a lower-reluctance external return path. In an air-core solenoid, flux that has left one axial end must return to the opposite end through air or surrounding space. That return path is long and high reluctance. High-permeability guides positioned along the applicator give part of that external return flux a preferred path. When properly spaced and sized, the guides reduce uncontrolled external spreading and support a stronger, more organized field through the treatment chamber. The guides can be passive or can be actively energized by one or more Flux-Guide Coils.

A Flux-Guide Coil energizes a longitudinal high-permeability return structure rather than the hollow treatment chamber. The Flux-Guide Coil can supply auxiliary magnetomotive force to the guide, magnetically bias or pre-magnetize the guide before a main treatment pulse, remain energized during at least part of the treatment pulse, be energized after the treatment pulse to shape field decay, or be placed in a disconnected, high-impedance, freewheel, clamp, or reset state according to the desired operating mode. In a preferred implementation, the Flux-Guide Coil is separately controlled by a controller or microprocessor and can be driven from the same nominal low-voltage supply as the treatment Coils, such as a 24 VDC supply, or from a different guide-coil driver.

The distal flux lens acts near the distal opening. Its function is to reduce sidewall escape and shape the field so that flux remains useful through the end of the treatment chamber. A flux lens is especially helpful where the solenoid terminates near the distal phalanges. It provides a magnetic boundary condition at the distal perimeter, encouraging field lines that would otherwise leave radially through the sidewall to continue toward the distal aperture.

The distal flux target provides a high-permeability destination beyond or adjacent to the distal aperture. It draws Core Flux toward the distal end and receives flux after it passes through or near the distal opening. When coupled to axial flux guides, the target becomes the distal collector of a return-yoke system. The target has enough cross-section to avoid localized saturation and is positioned with an appropriate gap so that it draws flux through the treatment region rather than short-circuiting the field before it reaches tissue.

The preferred practical embodiment is an integrated L-shaped guide-target member. A base plate extends along the bottom or another exterior side of the applicator from the proximal region toward the distal region, and an upright end plate extends across or near the distal end. The upright portion functions as a flux target, while the base portion functions as an axial return yoke. A single bent piece of low-carbon steel can therefore provide a significant distal-end improvement at low cost and with simple assembly.

The enhancements are individually useful and mutually reinforcing. Distal turn-density shaping supplies more local magnetic drive. Commingled or overlapping Windings smooth the axial ampere-turn distribution and can eliminate winding-free discontinuities that otherwise create leakage paths. The lens shapes the aperture. The target draws the field through the distal treatment region. The guide returns the field. A Flux-Guide Coil can actively assist or time-control the magnetic state of the guide. The integrated member reduces part count and improves magnetic continuity. Temperature-responsive pulse control uses available thermal headroom to increase distal output, reduce delivery time, or improve total magnetic-flux dosage when the hardware can safely accept it. The result is a distal-end therapeutic flux architecture rather than a collection of unrelated add-ons.

Temperature-responsive magnetic-flux optimization is likewise disclosed as a flux-enhancement feature rather than as merely a temperature cut-off. In a compact tapered solenoid applicator, the distal winding region will often be the hottest and therefore the region that limits the permissible treatment protocol. A fixed protocol must normally assume a high starting temperature and a correspondingly small thermal budget, which can substantially derate the delivered magnetic-flux dose. By measuring the actual applicator temperature, preferably including the distal Coil or a thermally coupled distal structure, the controller can use available thermal headroom to increase delivered dose, reduce treatment time, or increase the number of usable treatment sessions while remaining within a temperature limit.

### BRIEF DESCRIPTION OF THE DRAWINGS

FIG. 1 is a perspective view of a segmented solenoid applicator having axially aligned Coils arranged around a core that forms a treatment zone.

FIG. 2A depicts a spin-wound solenoid winding assembly having commingled winding portions from multiple separately energizable coil segments.

FIG. 2B illustrates the solenoid winding assembly of FIG. 2A disposed around an applicator core structure that defines a treatment chamber, with a human foot distally inserted and with the solenoid elongated beyond the treatment chamber reference region.

FIG. 3 is a side cross-sectional view of a segmented solenoid with a foot profile in the treatment zone, illustrating distal end-effect divergence of magnetic flux.

FIG. 4 is a side cross-sectional view of a segmented solenoid elongated beyond the distal reference region so that the natural end-effect region is displaced distally of the relevant treatment-chamber reference point.

FIG. 5 is a side cross-sectional view of a segmented solenoid having increased distal longitudinal turn density that still has distal end-effect divergence of magnetic flux.

FIG. 6 is a perspective view of a segmented solenoid applicator with exterior axial flux guides and a segmented distal flux lens.

FIG. 7 is a side cross-sectional view showing magnetic flux lines when axial flux guides and a distal flux lens are present.

FIG. 8 is a side cross-sectional view showing increased distal longitudinal turn density, exterior axial flux guides, a distal flux lens, and reduced end-effect divergence.

FIGS. 9A and 9B are perspective and sectional perspective views of an applicator with exterior axial flux guides, a distal flux lens, and increased distal longitudinal turn density.

FIGS. 10A and 10B are perspective and sectional perspective views of an applicator with increased distal longitudinal turn density, exterior axial flux guides, a distal flux lens, and a distal flux target plate.

FIG. 11 is a side cross-sectional view of an applicator with a distal flux target plate, distal target gap, flux lens, increased distal longitudinal turn density, and exterior axial flux guides, with flux lines illustrating the improved distal-end flux path.

FIG. 12 is an exploded perspective view of an applicator using bar-shaped axial flux guides, increased distal longitudinal turn density, a distal flux lens, and a distal flux target plate.

FIG. 13 is an exploded perspective view of an applicator using plate-shaped axial flux guides, a distal flux lens, and a distal flux target plate.

FIG. 14 is a perspective view of an applicator in which a strip, film, or plate provides a U-shaped guide-target structure.

FIG. 15 is a perspective view corresponding to FIG. 14 with magnetic flux lines added.

FIG. 16 is an exploded perspective view of the U-shaped guide-target embodiment with a distal target plate and guide strip.

FIG. 17 is an exploded perspective view of an applicator using an integrated L-shaped guide-target member.

FIG. 18 is a perspective view of the FIG. 17 components in assembled relationship.

FIG. 19 is a cross-sectional view of the L-shaped guide-target embodiment with magnetic flux lines.

FIG. 20 is a top view of a guide-target embodiment, illustrating the relationship among the treatment zone, the distal end, an axial flux-return path, and a guide-target member.

FIG. 21 is an assembled applicator view showing overlapping, commingled, or irregular Windings along the segmented solenoid.

FIG. 22 is an exploded perspective view of an applicator using an axial flux guide having one or more Flux-Guide Coils positioned on or around the flux guide to actively energize, magnetically bias, or precondition the flux guide during operation.

FIG. 23 is an internal side view illustrating wire-management features and electromagnetic coil-wiring routing within the rigid housing structure and coil supports of a segmented solenoid applicator.

FIG. 24 is a perspective view of a dual-foot, dual-chamber MPTD applicator as viewed from the distal end, with portions exposed to show internal winding assemblies, driver electronics, energy-storage capacitors, temperature sensors, and wire-management structures.

FIG. 25 is a Magnetic Flux Density Curve in millitesla over time for representative Coils of an MPTD applicator.

FIG. 26A illustrates a measurement model for determining solenoid elongation using treatment-chamber geometry, including candidate reference points relative to a proximal point of engagement, a treatment-chamber distal candidate point, and height-derived candidate points. In the illustrated example, several reference points align at a candidate point locus.

FIG. 26B illustrates the measurement model of FIG. 26A in a non-perspective cross-sectional view.

FIG. 26C is an illustrative table for a representative applicator showing chamber height and calculated relative distal distance for height-derived candidate points in one preferred embodiment.

FIG. 27 illustrates a patient using a planar plate applicator for a pulsed magnetic therapy session.

FIG. 28 illustrates a patient using two planar plate applicators for a pulsed magnetic therapy session.

FIG. 29 illustrates a patient using a planar plate applicator configured for treating two feet simultaneously during a pulsed magnetic therapy session.

FIG. 30 is a perspective view showing a single-foot treatment configuration.

FIG. 31 is a side view corresponding to FIG. 30, further illustrating the alignment of an inserted foot with the inclined top plate and flat bottom plate.

FIG. 32 is a perspective view of the internal structure, showing the top and bottom planar plates and the central magnetic core, excluding housing, electronics, and coils for clarity.

FIG. 33 is a detailed perspective view of the coils shown wound around the core between the two planar plates.

FIG. 34 is another perspective view of the coils shown wound around the core between the two planar plates.

FIG. 35 is a side view of the coils wound around the core and between the top and bottom plates.

FIG. 36 is an exemplary embodiment of a planar plate applicator with top and bottom plates replaced by side plates.

FIG. 37 is a perspective view of the planar plate applicator of FIG. 36.

FIG. 38 is a front-left perspective view of the internal structure.

FIG. 39 is back perspective view of the internal structure.

FIG. 40 is a graph or simulation output (if applicable) illustrating measured or simulated flux density across the treatment zone, correlating distance from the distal end with the resulting magnetic field strength.

FIG. 41 is an electrical schematic showing an exemplary driver circuit for powering the coil(s), including CPU, drivers, polarity switching, and coil gating.

FIG. 42 is an exemplary block diagram of the pulse generation system and feedback loop, indicating control logic, safety circuitry, and pulse sequencing architecture.

### DETAILED DESCRIPTION

The detailed description proceeds from the baseline solenoid to the enhanced distal-end system. This order is intentional. The enhancement structures make sense only after the reader understands the field behavior of the segmented solenoid and why the distal digital end of a foot chamber is the hardest region to treat well.

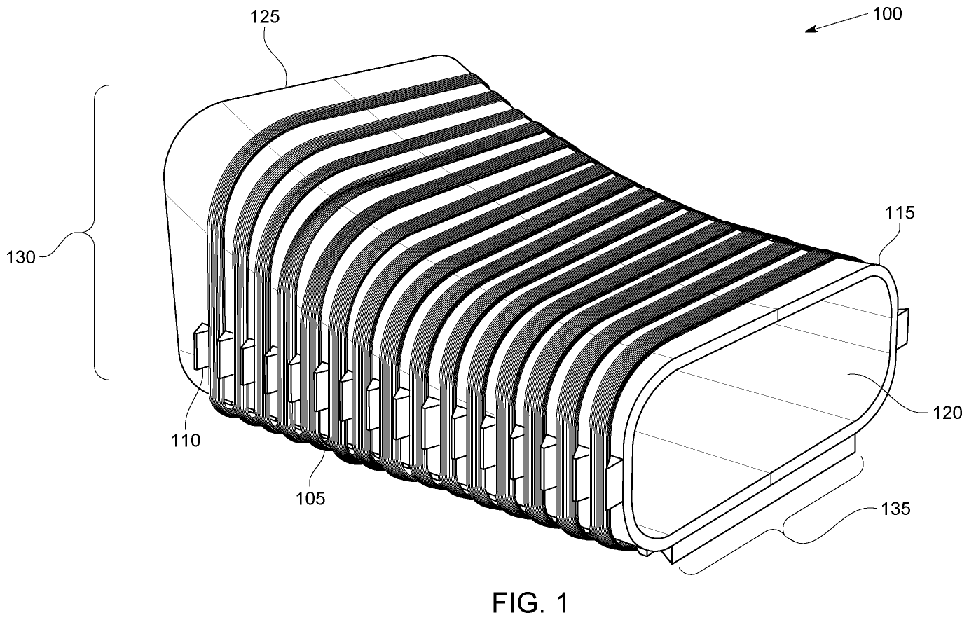

FIG. 1 illustrates an applicator 100 having a plurality of axially aligned Coils 105 separated by coil separators 110. The Coils collectively surround a hollow applicator core 115 that defines the treatment chamber; when a selected combination of Coils 105 is energized, the region of the chamber within the energized Coils forms treatment zone 120. A foot or other appendage is inserted through entry point 125 at proximal axial end 130 and advances toward distal end 135. In representative low-voltage embodiments, each Coil is wound from copper conductor, such as 18 AWG to 24 AWG wire, with the number of Turns selected for the available driver voltage, desired dB/dt, heat limits, and required Magnetic Flux Density.

The baseline structure is a segmented solenoid rather than a single continuous helix. Each Coil can be driven independently or in selected groups. When the driver energizes a selected combination of Coils with coordinated magnetic polarity, the Coils magnetically couple and form an Operative Solenoid. The treatment chamber is thereby placed within the core of the solenoid. This is the essential departure from surface-field applicators: the foot is placed inside the useful field rather than next to a field source.

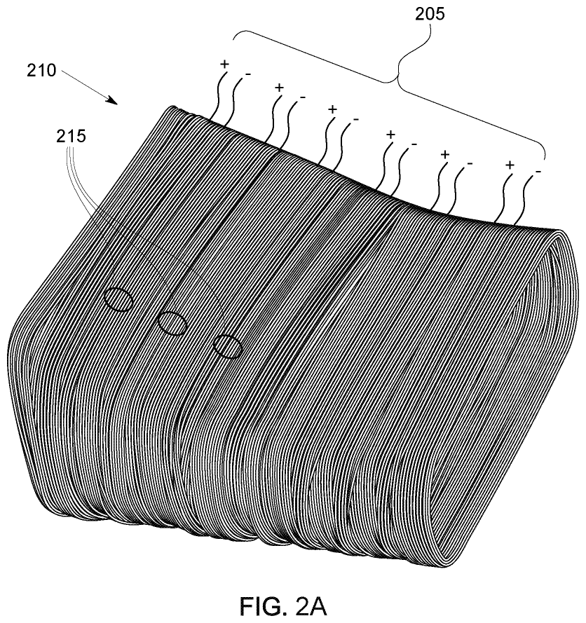

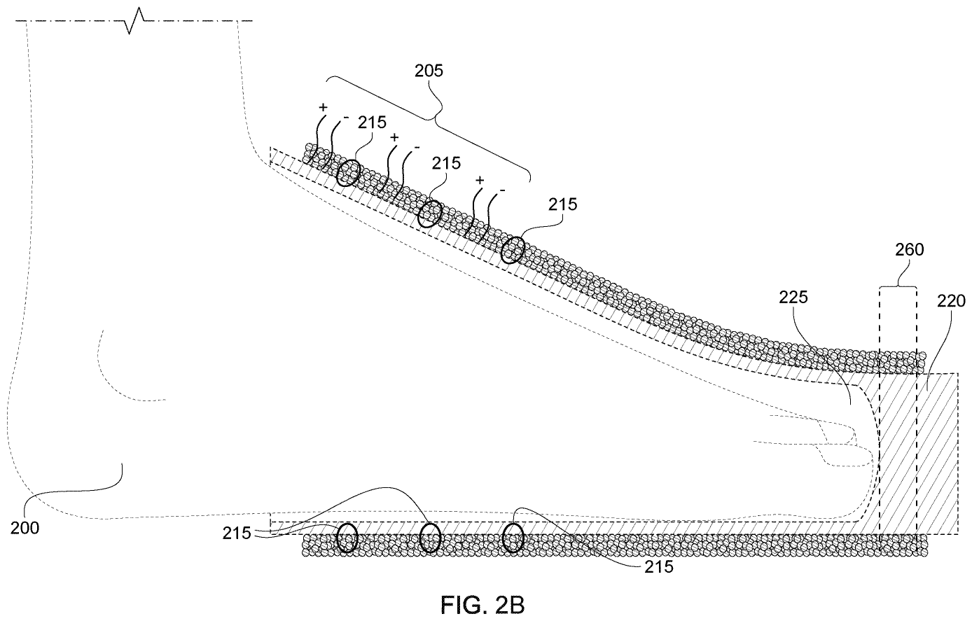

FIG. 2A illustrates a spin-wound solenoid winding assembly 210 before or apart from final housing integration. The winding assembly 210 includes a plurality of separately energizable coil segments 205 arranged along the proximal-distal direction. Adjacent coil segments 205 include commingled winding portions 215 that occupy common longitudinal positions within one or more longitudinal overlap regions. The commingled winding portions 215 are not uncontrolled loose wire; they are winding portions produced by a winding process and retained by the geometry and retention features of the applicator so that the coil segments remain separately energizable while the physical winding boundary between adjacent segments is magnetically smoother than a hard bobbin-to-bobbin boundary.

FIG. 2B illustrates the winding assembly 210 disposed around an applicator core structure 220. The applicator core structure 220 defines a treatment chamber 225 and supports the winding assembly 210 outside the user-facing chamber, with commingled winding portions 215 of adjacent coil segments 205 visible along the winding mass. A foot 200 is shown inserted distally into the treatment chamber 225. The winding assembly 210 extends distally beyond a reference region of the treatment chamber, forming an elongated distal winding portion 260. The same figure also illustrates how a tapered or sloped applicator core structure 220 can cause Windings of adjacent coil segments to nest and commingle while still being located by molded support features, retainers, dividers, barbs, or channels such as features 110 (FIG. 1).

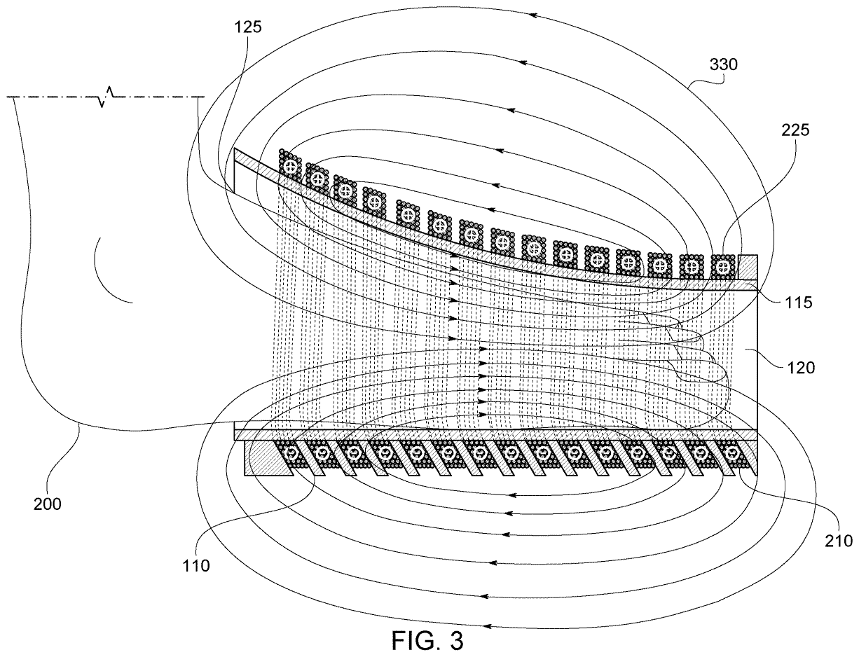

FIG. 3 shows the problem that remains. Magnetic flux 330 travels through treatment zone 120 but begins to diverge near distal end 135. In the side view, some field lines turn outward through the sidewall before reaching the distal-most end of the treatment region. In a foot applicator, that end corresponds to the distal phalanges. The field weakening is therefore not an academic artifact; it occurs at the anatomical region where peripheral neuropathy symptoms commonly begin and where focused treatment is especially valuable.

The field pattern in FIG. 3 can be understood using ordinary solenoid physics. In the central portion of a sufficiently long solenoid, the field inside the core is comparatively organized, and the external field is comparatively weaker. Near an end, however, the boundary condition changes. Flux cannot simply continue forever along the axis. It must close its path and return. Because the air outside the solenoid is available everywhere, some flux begins to leave before the geometric end of the Coil structure. The result is fringing, reduced axial field, and increased radial components.

The disclosed invention improves the magnetic circuit seen by this distal field. The goal is not to eliminate all external return flux; that would violate the closed-loop nature of magnetic flux. The goal is to make the useful portion of the field stay in the treatment chamber longer, pass through the distal treatment region more strongly, and return through intentional high-permeability structures rather than through uncontrolled air paths.

### Solenoid Elongation

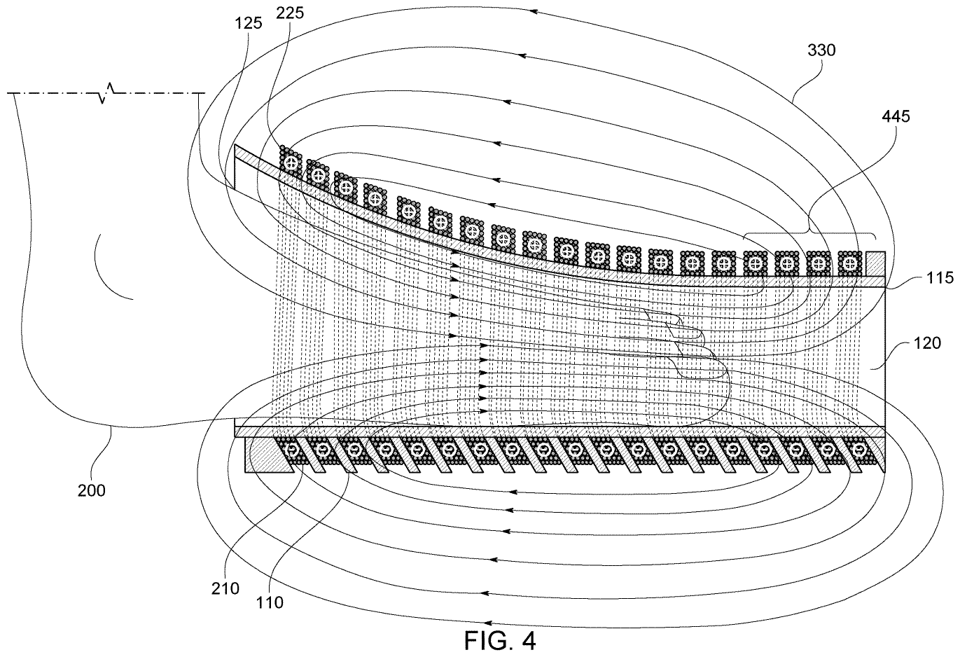

FIG. 4 illustrates the first and most direct solution: the solenoid is made longer than the treatment-chamber reference region used for distal treatment. Additional distal Coils 445 are positioned beyond the Reference Distal Point described below. The natural axial end-effect region still exists, but it is shifted distally of the RDP so that the relevant distal portion of the treatment chamber is not located exactly where the field begins to collapse.

In this disclosure, solenoid elongation can be defined by applicator geometry rather than by the actual anatomy of a particular user. The solenoid has a distal-most winding extent defined by the distal-most longitudinal position occupied by any Winding of the solenoid. In representative embodiments, the solenoid is elongated when the distal-most winding extent is positioned at least a selected distance distal of the RDP. A useful threshold is at least 10 mm distal of the RDP, although larger or smaller distances can be used when selected for treatment-chamber packaging, field uniformity, and measured distal Magnetic Flux Density.

The RDP is selected as the proximal-most point among a set of chamber-defined candidate points. Selecting the proximal-most candidate is conservative because it prevents a device from being characterized as elongated merely because one optional distal or proximal structure produces a more distal reference. The candidate points are based on the treatment chamber and chamber-engagement geometry, not on a measured foot in the chamber.

A first candidate is a Distal-Stop Derived Candidate Point. This candidate exists when the treatment chamber includes a distal-most structure configured to limit distal movement of the toes of a received foot. If the treatment chamber is open distally, or if the distal structure does not limit distal toe movement, this distal-stop-derived candidate may not exist and is not used for the RDP determination.

A second category includes Proximal-Stop Derived Candidate Points. In some applicators, one or more proximal portions of the treatment chamber may engage a medial ankle region, medial malleolar region, ankle-adjacent region, or lower-leg region and thereby limit further insertion of the foot. Each such proximal point of engagement is considered, and a corresponding candidate point is located 150 mm distal of that proximal point of engagement along the distal insertion direction. When this candidate category is used, and when several proximal points of engagement could contact the user, all corresponding proximal-stop-derived candidate points are included in the RDP candidate set.

The proximal-stop-derived candidate category need not be used in every RDP determination. In one embodiment, the RDP candidate set includes a distal-stop-derived candidate point when present and height-derived candidate points, without including proximal-stop-derived candidate points. In another embodiment, the RDP candidate set further includes the 150 mm proximal-stop-derived candidate points. In each case, the RDP is the proximal-most candidate point in the candidate set being applied.

A third category includes Height-Derived Candidate Points. These candidates account for cases in which the dorsum surface of a typical adult human foot would be the limiting engagement surface, but the calculation remains based on treatment-chamber geometry. For each corresponding longitudinal position along the treatment chamber between the proximal and distal ends of the chamber, the chamber height H is measured in millimeters at the longitudinal centerline of the chamber. A height-derived candidate point is then located distally of that corresponding longitudinal position by a distance D, in millimeters, where D=2.188H−37.1. The formula therefore uses chamber height as a geometric proxy and does not require measuring or relying on the actual anatomy of a particular foot.

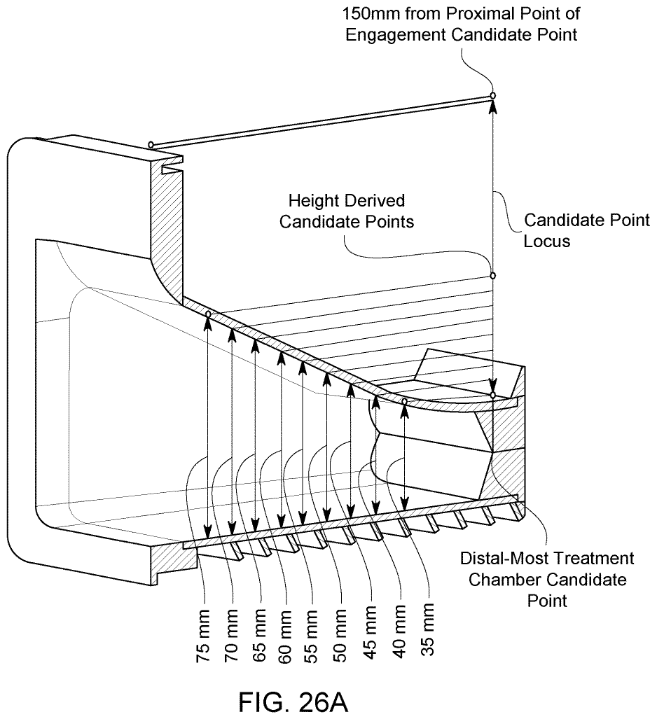

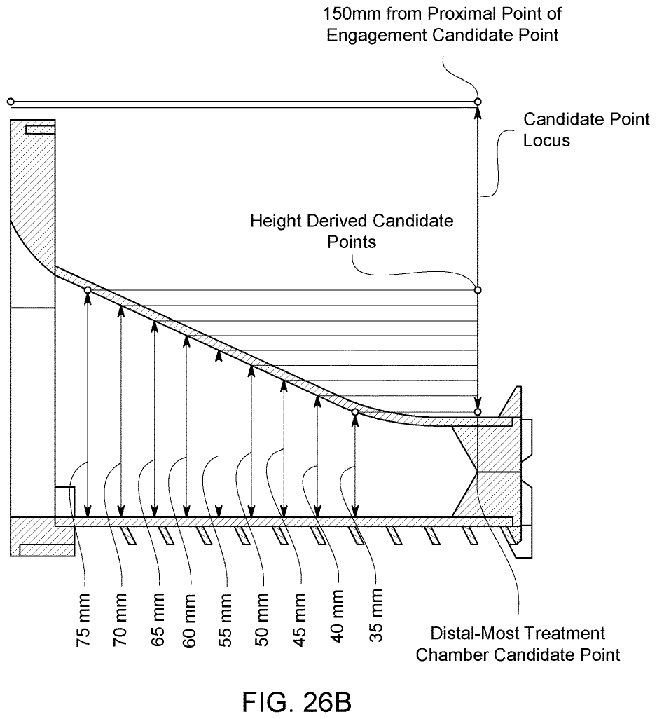

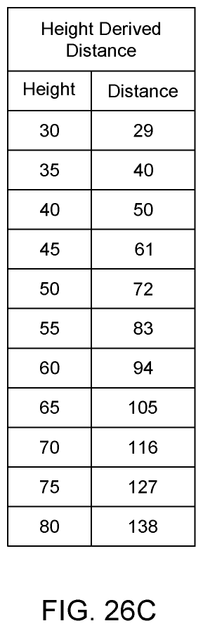

FIG. 26A illustrates the RDP measurement model in perspective. FIG. 26B illustrates the same model in a non-perspective cross-sectional view. FIG. 26C provides an illustrative table for a representative applicator, showing chamber heights and calculated relative distal distances for height-derived candidate points. In the illustrated example, the distal-stop-derived candidate, a proximal-stop-derived candidate, and selected height-derived candidates happen to align near a candidate point locus. That alignment is not required; when the candidate points differ, the RDP is the proximal-most candidate point among them.

Elongation is robust because it does not rely on subtle material properties. Its cost is length, added Coils, added driver resources, and added conductor resistance. In a compact forefoot product, this cost can be significant. The preferred design therefore treats elongation as one available tool rather than as the only answer. A small amount of elongation combined with flux lensing, targeting, external guiding, distal turn-density shaping, or commingled winding construction often provides a better cost-volume-performance tradeoff than elongation alone.

In representative embodiments, the active Coil set extends at least one Coil pitch beyond the RDP. Other embodiments extend two, three, four, or more Coil pitches beyond the RDP. The distal extension need not be an integer number of complete Coils. It can be provided by any number of additional Turns, Windings, partial winding portions, complete Turns, complete Coils, partial Coil segments, or combinations of those structures positioned distal of the RDP. The selected elongation distance is driven by measured distal Magnetic Flux Density, dB/dt, thermal behavior, driver resources, and treatment-chamber packaging rather than by an arbitrary number of added segments or by the dimensions of a particular user's foot.

### Distal Longitudinal Turn-Density Shaping

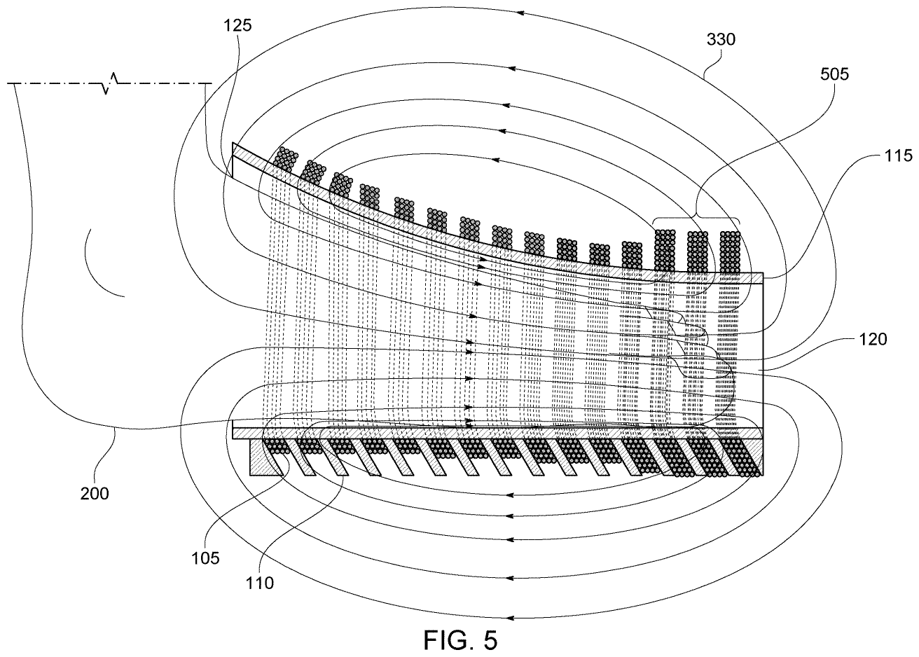

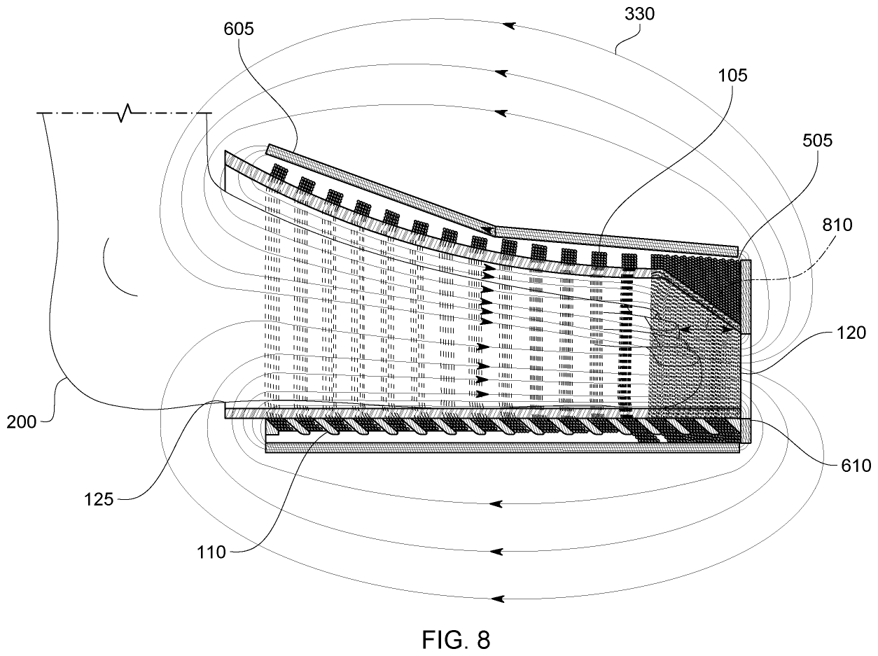

FIG. 5 and FIG. 8 show Longitudinally Increased Coil Density (“LICD”) 505 near distal end 135. LICD means that a distal winding-density region has a greater number of winding Turns per unit longitudinal length than a proximal reference region, preferably when the distal winding-density region and proximal reference region are measured over equal longitudinal lengths. The point is not merely to add copper. The point is to increase local ampere-turns per unit length where the finite solenoid would otherwise lose axial field. In a simple approximation, Magnetic Flux Density inside a long air-core solenoid is proportional to ampere-turns per unit length. Increasing distal turns therefore increases the local magnetomotive force available to support the distal field.

In density-defined embodiments, the distal winding-density region can have a longitudinal length of at least 25 mm measured along the distal insertion direction, and the proximal reference region can have the same longitudinal length as the distal winding-density region while being proximal to and non-overlapping with it. In representative embodiments, the longitudinal winding density of the distal winding-density region is at least 20% greater than the longitudinal winding density of the proximal reference region. Each winding density can be determined from complete Turns and portions of Turns of one or more Coils that occupy the respective region, divided by the longitudinal length of that region.

The design must be balanced because added Turns also increase inductance. A Coil driven from a fixed voltage does not instantly reach its final current; the current rise is governed by the electrical time constant and driver characteristics. Adding turns can increase the final ampere-turn capability but slow the current rise and reduce dB/dt during the portion of the pulse that matters. Thus, a useful LICD arrangement is not simply the maximum possible number of Turns. It is a geometry that increases distal field support while preserving a fast enough magnetic pulse.

In FIG. 5, layered distal Windings increase distal conductor density within the LICD region 505. In FIG. 8, triangular or tapered distal winding packing 810 places additional conductor near the distal sidewall while reducing unnecessary mutual inductance. The geometry can be implemented by shorter distal Coils, added overlapping Windings, different wire gauge, different pitch, or additional localized Windings. The engineering test is whether the distal treatment region receives improved B(t), peak B, or dB/dt under the relevant same-test condition without unacceptable heating.

LICD also complements the flux lens and target. The lens and target influence where the flux prefers to travel, but they do not create magnetomotive force by themselves. Distal turn-density shaping supplies more local excitation, while the high-permeability members give that excitation a better magnetic circuit. The combination is therefore more effective than either feature alone.

### Commingled Windings and Segment-Length Control

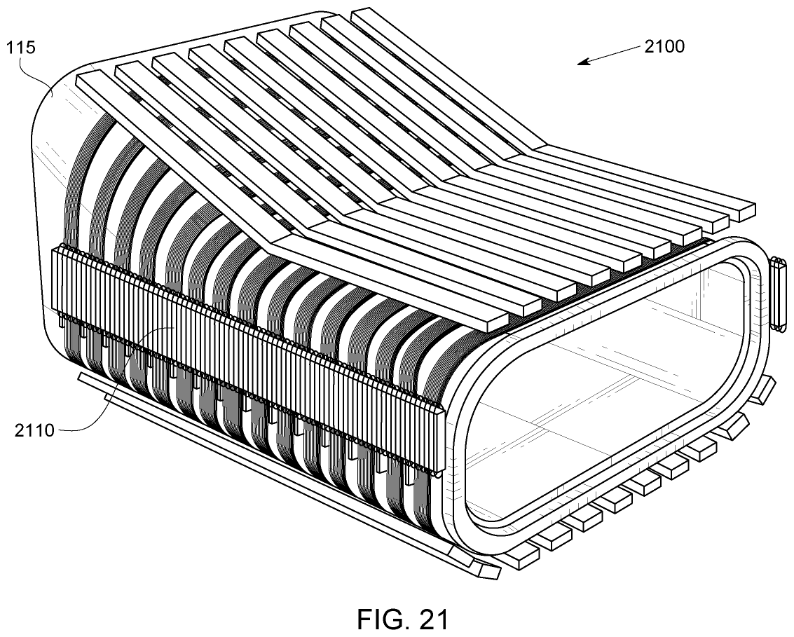

FIG. 21 depicts an assembled applicator 2100 in which separately energizable coil segments 2110 include overlapping, irregular, or commingled Windings along the solenoid length. FIGS. 2A and 2B further illustrate a spin-wound implementation in which adjacent coil segments 205 have commingled winding portions 215. This construction is a deliberate MPTD segmented-solenoid enhancement rather than merely a tolerance condition or manufacturing imperfection.

A conventional segmented solenoid is often imagined as a series of sharply separated coils. The coils may be wound on separate bobbins, divided by separator plates, stacked as isolated modules, or retained in precisely bounded winding channels. Such construction can be useful and remains within the broader disclosure. It is especially understandable in linear actuators, where discrete segment coils are commonly used to position or accelerate a movable armature and where a hard axial field boundary can help define a longitudinal force position.

The disclosed MPTD applicator has a different functional requirement. The magnetic flux acts directly on tissue disposed in the treatment chamber rather than on a movable ferromagnetic armature that must be positioned at a discrete axial location. In this environment, a sharply separated boundary between adjacent coil segments, although a fully usable construction within this disclosure, is less preferred for field smoothness. Depending on geometry, such a boundary can present a local step or dip in the longitudinal ampere-turn distribution, an abrupt field-gradient change, a localized axial field variation, or an inter-segment flux transition in the treatment chamber that commingling reduces.

Controlled commingling reduces these effects. Adjacent separately energizable coil segments can remain electrically and functionally distinct while their winding portions are both present at common longitudinal positions. The overlap may be limited to a portion of a turn path, such as the side of the solenoid corresponding to an upper boundary of the treatment chamber, or may extend around a larger portion of the solenoid. The disclosure does not require an entire Turn of one Coil segment to overlap an entire Turn of another Coil segment.

The longitudinal overlap region can be selected by design. Representative continuous overlap regions include at least 3 mm, at least 5 mm, at least 10 mm, or another value selected for field smoothness, conductor placement, manufacturability, and desired segment resolution. In some embodiments, at least four sequential coil segments each overlap with an adjacent coil segment along a side of the solenoid corresponding to the upper boundary of the treatment chamber.

An actual commingling zone can be defined where Windings of two adjacent separately energizable Coil segments occupy overlapping longitudinal positions. The actual commingling zone can have a longitudinal extent of at least 3 mm, at least 5 mm, at least 10 mm, or another selected extent measured along the longitudinal direction of the solenoid. In some embodiments, within the actual commingling zone, at least one winding portion of one Coil segment is positioned at least 3 mm distal of at least one winding portion of the adjacent Coil segment, demonstrating true longitudinal interpenetration rather than mere end-to-end contact.

Commingling can also occur through the thickness of the winding mass. For example, within an actual commingling zone, a winding portion of one Coil segment can be positioned between winding portions of an adjacent Coil segment when measured through the winding-mass thickness outward from the treatment chamber. Such interleaving can occur while the Coil segments remain electrically separate and separately energizable.

Magnetic advantages of commingled winding portions 215 include smoothing the axial ampere-turn distribution, reducing field discontinuities at segment boundaries, improving longitudinal flux coherence, reducing radial sidewall escape caused by segment-boundary gaps, improving magnetic coupling between adjacent selected coil segments, smoothing transitions into distal winding-density regions, reducing sensitivity to turn-placement tolerance, and allowing compact longitudinal packing without sacrificing separately energizable control.

Controlled commingling is a deliberate and unexpected performance enhancement rather than a mere tolerance condition. In a segmented solenoid, the transition between adjacent coil segments can otherwise produce a local disturbance in the longitudinal field. Commingling blends and softens the flux transition between adjacent coil segments so that the longitudinal field varies smoothly across each segment boundary. Expressed in measurement terms, when a magnetic probe is passed along the length of the solenoid, a commingled construction presents no spikes or dips in Magnetic Flux Density at the coil-segment boundaries: there is neither a gap where the field falls off between segments nor a sharp local reinforcement where segments meet. Commingling therefore produces higher-quality flux along the treatment zone. This benefit is in addition to the distal flux enhancements described herein—including solenoid elongation, distal longitudinal turn-density shaping, the flux lens, the flux target, and the axial flux guides—and does not degrade coil performance. In particular, commingling does not reduce the effectiveness of Dynamic Directional Flux Control (DDFC), because DDFC depends on electrical segmentation and controlled polarity rather than on a physically sharp boundary between adjacent Windings. Commingling further distinguishes the present segmented solenoid from the CN '815/'524 Reference, which discloses discrete local perimeter coils assigned to separate anatomical regions and does not disclose Windings of two or more separately energizable coil segments that intermingle at common longitudinal positions to blend the longitudinal field of a common Operative Solenoid.

Commingling is particularly useful where a distal winding-density region transitions from a lower-density region to a higher-density region. Rather than requiring an abrupt change in conductor density, the Windings can transition gradually. This can reduce local field nonuniformity, improve use of available chamber length, and allow conductor density to increase toward the distal region without creating a magnetic step change at a single axial boundary.

The construction also distributes conductor transitions and heat more gradually. A sharply separated segment boundary can concentrate wire crossovers, lead exits, tension changes, and mechanical stress at a small location. A commingled transition can spread those changes over a longitudinal interval, which can reduce localized conductor stacking, reduce localized thermal concentration, and improve winding stability during repeated pulsing and thermal cycling.

The stair-stepped construction problem is significant enough to deserve separate treatment. In a layered or stair-stepped solenoid, each step creates a sidewall discontinuity, and the vertical or sloped surfaces between steps often leave gaps large enough for magnetic flux to exit the winding mass instead of remaining organized through the treatment chamber. The leakage is commonly most pronounced at the portion superior to the treatment chamber, where the winding stack steps away from the chamber wall and where no continuous tapered sidewall keeps the return path disciplined. The gaps therefore allow flux to leak, become Non-Core Flux, and return through air before contributing usefully to distal treatment. A benefit of the tapered treatment-chamber core structure is that it removes or materially reduces these sidewall stair-step leakage paths, so the Windings can follow a smoother chamber-supporting contour rather than presenting open step gaps to the field.

In preferred superior-side constructions, the winding mass is continuous along the superior side region across an interface between adjacent Coil segments, without a winding-free discontinuity between adjacent portions of the winding mass. The avoided discontinuity includes a winding-free discontinuity formed by a stair-step transition between axially adjacent winding stacks, including vertical or inclined step faces that may not appear as an open slot in a top plan view. This statement concerns gaps or discontinuities in the winding mass between segment regions and does not exclude the normal microscopic interstitial spaces between adjacent round or insulated conductors.

Controlled commingling does not defeat Dynamic Directional Flux Control. DDFC depends on electrical segmentation, controlled polarity, and selected energized or unpowered segment states, not on a visually sharp physical boundary between adjacent Windings. DDFC can perform well with commingled Windings, including operating patterns in which even one unpowered coil segment is positioned between regions energized with opposing flux polarities. The unpowered segment can still provide an electrical and magnetic transition interval while the commingled physical construction avoids unnecessary hard-boundary field harshness.

Segment length also affects performance. Shorter Coils concentrate magnetomotive force over a shorter length but increase the number of segments and can increase mutual inductance and driver complexity. Longer Coils simplify construction but reduce spatial control. In a representative low-voltage foot applicator, segment lengths around 10 mm to 25 mm are practical, with shorter or longer lengths selected for particular regions. The distal region can use different segment length than the proximal region because the distal treatment problem is different.

Commingled Windings, distal longitudinal turn-density shaping, and segment-length control are conductor-side shaping tools. Flux guides, lenses, and targets are magnetic-path shaping tools. The disclosed applicator uses both categories. The conductor tools decide where magnetomotive force is produced and how smoothly it changes along the chamber. The magnetic-path tools decide where flux is encouraged to travel after it is produced.

### Spin-Wound Tapered-Core Construction

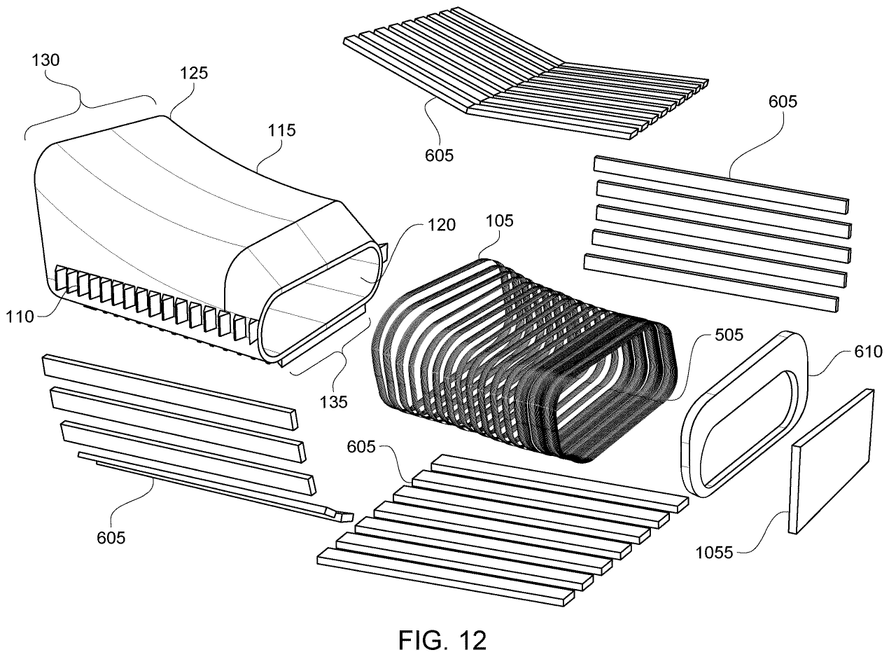

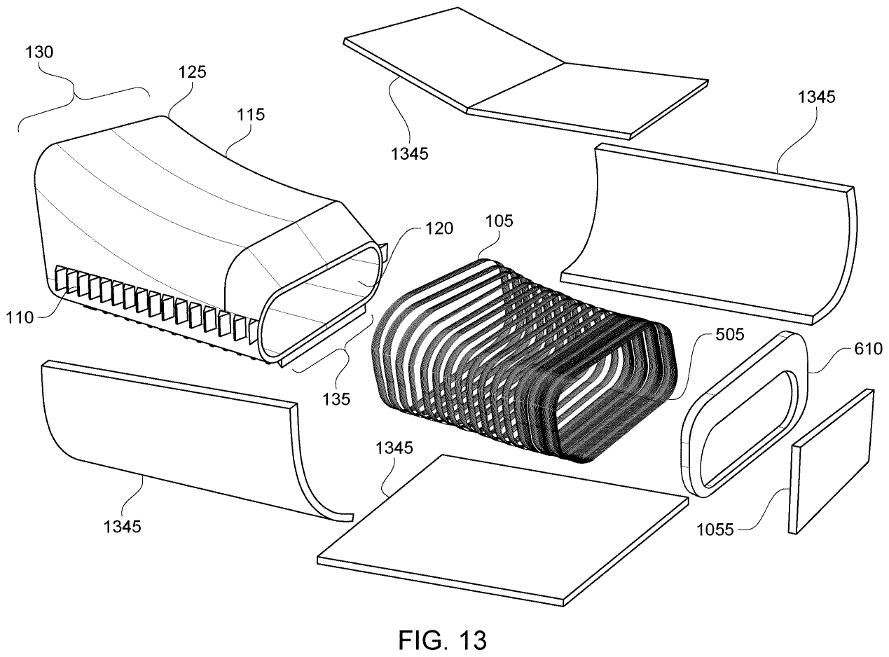

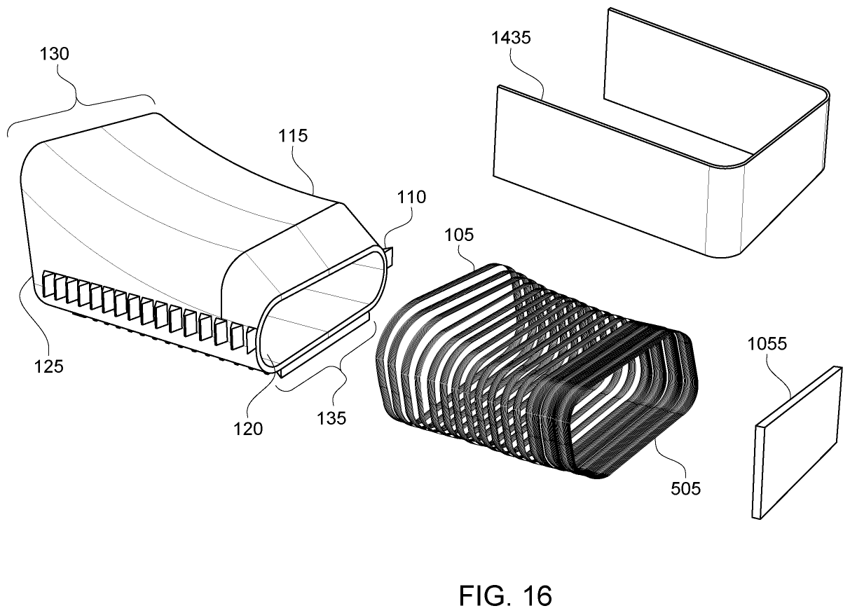

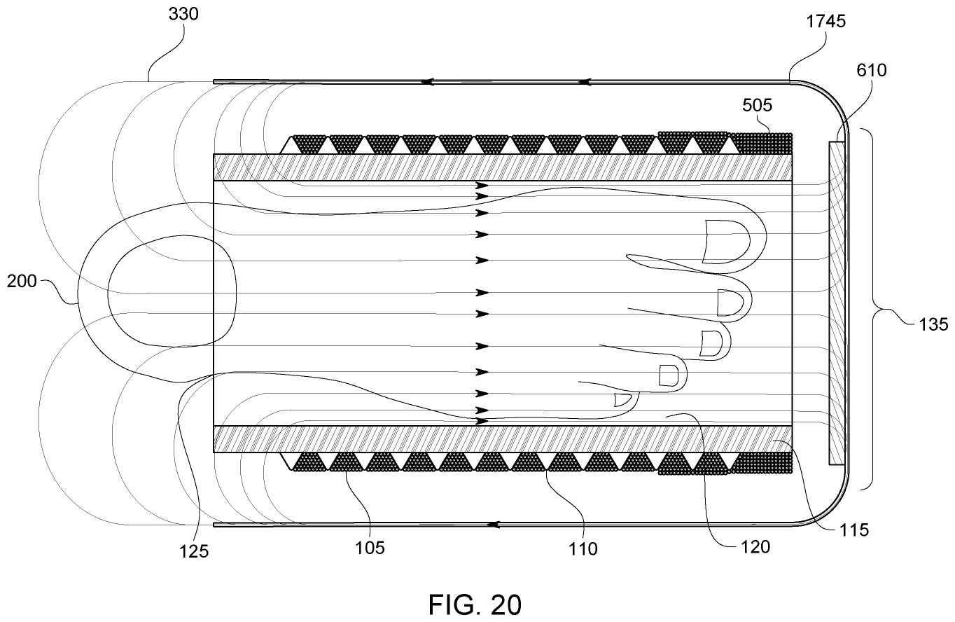

A preferred way to produce controlled commingling is to spin wind the coil segments on a tapered or sloped applicator core structure. Suitable sloped core structures are illustrated, for example, in FIG. 2B, FIG. 12, FIG. 13, and FIG. 16. The resulting wound assembly can resemble the assembled winding constructions shown in FIG. 20, FIG. 21, or related assembled applicator views, depending on the presence of flux guides, guide-target members, and production wire-management features.

Tapering of the chamber-supporting core is not merely a packaging detail. The superior winding-support portion can follow the rising dorsal or instep profile of a received foot so that Windings remain close to the treatment target over a long proximal-distal extent. This closeness improves magnetic coupling to the treatment region, and the gradual taper can produce a desirable proximal-to-distal winding and field gradient instead of a series of abrupt geometric steps.

In an objective taper definition, a superior winding-support portion can extend along a taper-measurement extent of at least 100 mm measured along the distal insertion direction. Over that taper-measurement extent, a chamber height measured at a longitudinal centerline between an inferior boundary of the treatment chamber and a superior boundary of the treatment chamber can be at least 30 mm throughout the extent and can increase by at least 40 mm from a distal end of the extent to a proximal end of the extent. Viewed in a longitudinal cross-section through the longitudinal centerline, the superior boundary of the treatment chamber can define a curved profile over at least 50 mm of the taper-measurement extent, and the local rate of chamber-height increase along the curved profile can be nondecreasing when moving proximally.

In one preferred process, winding begins at the distal end. The treatment-chamber core structure is slid onto a similarly shaped spindle, mandrel, or fixture and is rotated. A spool of insulated copper conductor, typically about 20 AWG in representative embodiments, supplies wire while the core rotates. The first coil segment, Coil 1, is spun wound at the distal end. The starting and ending conductor portions are tucked into or retained by plastic retainers, dividers, support channels, sidewall barbs, or wire-management structures 110.

After Coil 1 is wound, Coil 2 is wound in a similar manner. The process proceeds coil by coil toward the proximal end, such as through Coil 11 in a preferred eleven-coil embodiment. Each coil segment has its own electrical start and finish and remains separately energizable even though its winding portions can overlap, nest, or commingle with winding portions of adjacent coil segments.

The tapered chamber geometry assists the process. Each subsequent coil starts at its designated starting region, which can be indicated by retainers, dividers, channels, or barbs 110. Because the treatment-chamber core is tapered, the Windings tend to slide, settle, and pack distally against previously wound turns. As additional Windings build up, the conductor layers naturally compact against each other while still progressing proximally from segment to segment. Later coil Windings increasingly lock earlier Windings in place as the taper and the conductor stack cooperate.

This self-packing behavior produces controlled commingling without requiring a machine to place each turn with motor-style precision. The Windings tend to locate themselves in the correct lower support channel and between the sidewall barbs or retainers 110, even at production winding speeds. On the upper side of the chamber, the conductors can flatten, spread, and build into a compact winding mass that is superior to the treatment chamber and that has fewer hard longitudinal gaps than a stair-stepped discrete-coil construction.

Conventional coil-design practice often gives great attention to perfectly adjacent turn placement. That practice may be valuable in motors, high-frequency coils, linear actuators, and other devices in which local inductance, capacitance, impedance, armature force, or radio-frequency behavior is critical. In the disclosed low-frequency pulsed MPTD applicator, repeated high-precision magnetic-field measurements have shown negligible practical flux-performance advantage from tedious visually perfect turn placement when compared with high-speed spin-wound winding placement that preserves the intended electrical segmentation and chamber geometry.

The observed practical result can be opposite the conventional expectation. A stair-stepped coil construction—one in which sharply separated layered segments present visually clean boundaries—is characterized by sidewall steps and inter-step gaps through which flux can leak or short-circuit through the sidewall region before contributing usefully to the treatment chamber. The vertical or inclined face at each step, together with the gap associated with it, is a property of the stair-step geometry itself: on a distal-to-proximal probe sweep, such faces and gaps tend to produce flux anomalies that appear as spikes or dips in the longitudinal field. A tapered core structure closes these stair-step leakage paths. The tapered surface can initially create winding-migration challenges if the turns are wound as isolated, non-overlapping segments.

The tapered, spin-wound construction also improves the quality of Core Flux by promoting a more continuous winding mass. Eliminating winding-free gaps between Coil segments and eliminating stair-step faces reduces leakage and flux anomalies; even relatively small winding-free discontinuities can provide local paths for flux to leave the core region and become Non-Core Flux before it has contributed usefully to the treatment region.

Controlled spin winding with overlapping coil segments solves that manufacturing and magnetic problem. The winding portions lock to each other, remain tight, resist distal migration, resist unwinding or unfurling during handling and storage, and reduce audible vibration from loose conductors. The structure therefore improves manufacturing speed and winding stability while also reducing sidewall flux leakage paths. Sidewall barbs, lower support channels, and aligned support structures 110 further make the process repeatable by guiding each coil into the correct region during high-speed winding.

The mechanical benefit is related to the magnetic benefit. As winding proceeds from distal to proximal, later proximal Windings can layer over portions of earlier distal Windings, mechanically interlocking the winding mass. This interlocking helps the wound mass remain tight, resists distal sliding on the taper, reduces looseness during handling and pulsing, and allows spin winding to be fast and economical without requiring separate hard bobbin boundaries between adjacent Coil segments.

The construction also avoids the tooling burden of highly separated segmented coils. A discrete-segment design can require individual bobbins, separator plates, stacked coil modules, or carefully molded winding compartments. If those features are integrated into an irregular applicator core, the entire core may need to be handled by a complex winding machine that either encircles the non-cylindrical core or spins the core itself while managing imbalance and loose conductors. Controlled commingling allows a simpler spin-winding operation, faster cycle time, fewer precise start-stop boundaries, fewer handling problems, and less rework.

The resulting tapered, spin-wound, commingled construction can provide higher measured treatment-chamber Magnetic Flux Density B in representative embodiments, reduced sidewall leakage, less pulsing noise from Windings, less winding migration, less manufacturing rework, improved storage and handling stability, and improved thermal behavior relative to perfectly separated stair-stepped coil segments. The improvement is not uncontrolled winding placement. Rather, for this MPTD, the relevant precision is chamber geometry, electrical segmentation, winding retention, and controlled overlap, rather than visually perfect isolated turns.

### Magnetic-Circuit Principles for the High-Permeability Structures

The flux guide, flux lens, and flux target are all based on the same magnetic-circuit principle. Air has low permeability and therefore high reluctance. Ferromagnetic materials such as low-carbon steel, pure iron, silicon steel, nickel-iron alloy, and ferrite can have much lower reluctance while they remain below saturation. A high-permeability member placed at the right location changes the field distribution because flux tends to follow paths that reduce total magnetic reluctance.

The common magnetic-circuit relation is useful: flux is driven by magnetomotive force and opposed by reluctance. Reluctance increases with path length and decreases with permeability and cross-sectional area. This means that a long air return path is expensive magnetically, while a short, adequately sized steel or ferrite return path is cheaper. The analogy is not perfect because magnetic fields occupy space and are not confined to wires, but it is a good design guide.

High permeability is not unlimited. If a guide or target is too thin or has a narrow bend, the flux density inside that part can approach saturation. When saturation occurs, incremental permeability collapses and the member no longer behaves like an effective low-reluctance path. Thus, the cross-section, bend geometry, material selection, and air gaps are functional features. The best member is not necessarily the highest-permeability material; it is the member that remains effective, manufacturable, thermally acceptable, and unsaturated during the intended pulse regime.

Conductive ferromagnetic members also experience eddy currents when exposed to rapidly changing flux. Eddy currents can reduce field penetration into the member, delay the intended magnetic response, and create heat. In many representative MPTD embodiments, the field is in the tens of millitesla and the pulses are in the microsecond range, so a practical low-carbon steel member can still perform well. For higher dB/dt or thicker members, slots, laminations, insulated layers, ferrite, powdered magnetic material, or reduced conductive loop area can be used to reduce eddy-current effects.

### Axial Flux Guides

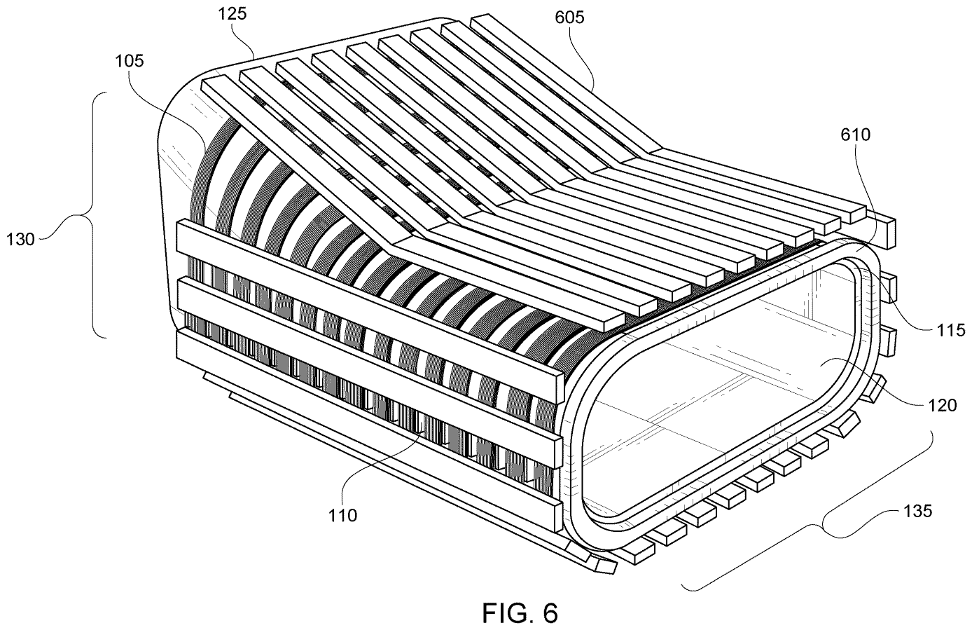

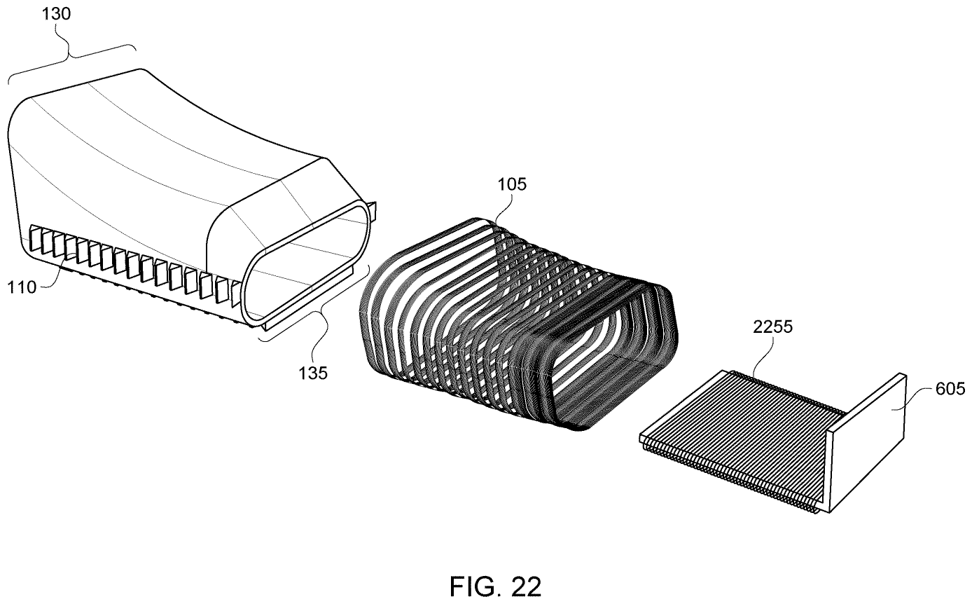

FIG. 6 shows axial flux guides 605 positioned along the outside of the applicator, together with a segmented distal flux lens 610, described below, arranged about the distal aperture. FIG. 22 shows a related embodiment in which at least one axial flux guide 605 carries or is electromagnetically coupled to a Flux-Guide Coil 2255. An axial flux guide 605 may likewise carry or be electromagnetically coupled to a Flux-Guide Coil 2255 in a bar-guide or side-guide arrangement. In an unenhanced solenoid, flux leaving the distal region returns to the proximal region through air and surrounding structures. That return path is diffuse and high reluctance. Axial flux guides create preferred external paths. They do not make the external field disappear, but they collect and organize part of the return field and reduce uncontrolled spreading.

In the illustrated implementation, the guides extend generally between distal end 135 and proximal end 130 along the outside of the Coils 105 and separators 110, with the functional return path running from distal to proximal after flux is collected near the distal region. They are spaced from the treatment chamber so that they guide return flux without becoming an internal iron core that would occupy the space needed by the foot. Suitable guide forms include bars, strips, rods, plates, laminations, stamped members, ferrite pieces, low-carbon steel members, silicon-steel laminations, or composite magnetic members.

An exterior side region of the solenoid can be a lower or inferior side region, a medial side region, a lateral side region, a superior side region, or another exterior region outside the treatment chamber. In a foot applicator, medial and lateral are defined relative to a foot received in the chamber, while a lower side region can include the underside or base region used by an L-shaped guide-target member. Thus, the term exterior side region is not limited to left and right sidewalls and includes bottom or lower return-yoke locations.

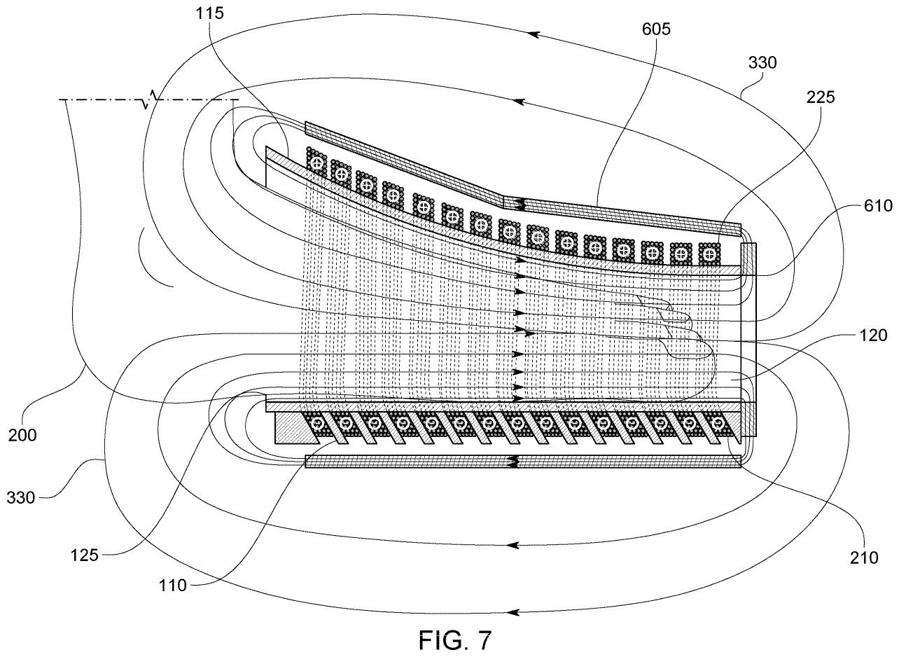

FIG. 7 shows the placement compromise by showing flux lines that would otherwise diverge from the distal sidewall being captured by correctly positioned guides and then carried along an exterior longitudinal return path, with a distal flux lens 610 also present at the distal end. A guide placed too far from the solenoid has little effect because the return field does not couple strongly to it. A guide placed too close, or placed in a way that bridges the wrong regions, can shunt field away from the treatment chamber. The correct position is an engineering compromise: close enough to provide a lower-reluctance return path, yet spaced and oriented so that the main treatment field remains in the hollow treatment chamber.

Bar-shaped guides, as shown in FIG. 12, are simple and leave air spaces between adjacent guide members. Those air spaces can reduce eddy-current loops and allow mechanical flexibility in assembly. In representative sidewall embodiments, two or more separate guides on each lateral side of the solenoid are preferred over one large sidewall plate, and guide widths in the range of about 7.5 mm to about 15 mm, including 15 mm or less, keep eddy-current effects minimal in compact pulse designs. Plate-shaped guides 1345, as shown in FIG. 13, provide greater cross-sectional area and lower magnetic reluctance, but they can create larger conductive loops and require more attention to eddy currents and saturation. The choice between bars and plates therefore depends on pulse duration, dB/dt, desired flux return, heat, cost, and manufacturability.

The axial guides also provide a convenient interface for other distal-end structures and for active guide energization. A distal flux target can collect flux at the distal opening, and the axial guides can carry that collected flux back toward the proximal region. A Flux-Guide Coil can be added to one guide, several guides, all guides, or an integrated guide-target member when active guide participation is desired. This division of labor is useful: the target handles the difficult end-turning region, the guides handle the long return path, and the guide coils can control how strongly or when that return path participates.

### Flux-Guide Coils and Active Guide Energization

FIG. 22 depicts an embodiment in which a Flux-Guide Coil 2255 is disposed on or around a longitudinal high-permeability member located external to the hollow treatment chamber and treatment zone 120. The Flux-Guide Coil 2255 can be applied to any suitable longitudinal ferromagnetic structure, including an axial flux guide 605, a bar-shaped guide, a plate-shaped guide 1345, a U-shaped guide-target member 1435, an L-shaped guide-target member 1745, the base portion of an L-shaped member, a side guide, a bottom guide, or another high-permeability return-yoke structure.

The purpose of the Flux-Guide Coil 2255 is broader than pre-biasing. The coil can actively energize the flux guide so that the external return path participates more strongly, earlier, or in a selected polarity relationship during a magnetic pulse. In one operating mode, the coil supplies auxiliary magnetomotive force to the guide and thereby assists movement of flux between the distal end region and the external longitudinal return path. In another operating mode, the coil magnetically biases or pre-magnetizes the guide before the treatment Coils are energized, so that the guide begins the main pulse at a selected magnetic operating point rather than at an uncontrolled residual state.

The magnetic effect of guide energization is selected empirically and is not assumed to be beneficial merely because current is present. A useful bias or active guide current can increase coupling of the guide to the desired return path, improve early-pulse guide participation, reduce magnetic lag associated with domain motion or hysteresis, increase distal Magnetic Flux Density, reduce radial divergence, or improve repeatability of B(t). Excessive guide excitation can instead move the guide toward saturation, reduce incremental permeability, increase heat, increase eddy-current loss, or pull flux away from the treatment chamber. For that reason, guide-coil current, timing, polarity, and duty cycle are design variables rather than fixed requirements.

The Flux-Guide Coil 2255 can be implemented as a single Winding around one guide, multiple Windings around respective guides, a distributed Winding along a plate, a bobbin-supported Winding surrounding a guide, a Winding captured by a housing channel around a guide, or separate guide coils driven independently. In a cost-effective embodiment, the Flux-Guide Coil 2255 is positioned on the base of an L-shaped guide-target member 1745 or on two bar-shaped axial flux guides located on opposite sides of the applicator exterior. Other embodiments energize only a distal portion, only a proximal portion, or selected intermediate portions of a longitudinal guide.

The Flux-Guide Coil 2255 is preferably electrically separate from the treatment Coils 105 and separately controlled by a controller or microprocessor. It can be driven from the same nominal supply used by the treatment Coils, such as a 24 VDC supply, or from a different voltage or current source. Suitable guide-coil drivers include low-side switches, half bridges, H-bridges, current-regulated drivers, capacitor-supported pulse drivers, pulse-width-modulated drivers, or other circuits capable of controlling current magnitude, timing, and polarity.

The controller can energize a Flux-Guide Coil 2255 before a selected treatment pulse, concurrently with the treatment pulse, after the treatment pulse, during only a portion of a pulse, during selected pulses in a pulse train, or during a reset interval. The controller can also de-energize or turn off the Flux-Guide Coil 2255 between at least some magnetic pulses. A pre-bias interval can be used to establish a selected magnetic state in the guide before the main solenoidal field rises. A concurrent interval can actively reinforce guide participation during the treatment pulse. A post-pulse interval can shape collapse of the return flux, remove residual magnetization, reverse a prior bias, or prepare the guide for an opposite-polarity pulse.

Polarity control is important in embodiments that reverse solenoid polarity, use bipolar pulses, or use Dynamic Directional Flux Control. The guide-coil polarity can track the polarity of the selected treatment Coils, oppose it, remain fixed for a selected interval, alternate from pulse to pulse, or be driven through a demagnetizing or reset sequence. The correct relationship is determined by the desired magnetic path. In a simple full-solenoid operating mode, the guide coil will commonly be driven so that the guide assists longitudinal return flux. In a directional mode, the guide coil can be disabled or driven differently so that it does not force flux into an unwanted return path.

The guide-coil driver can provide several electrical states. During active drive, the driver forces current through the Flux-Guide Coil 2255. During controlled freewheel or recirculation, current already present in the guide coil is allowed to continue and decay through a selected path, which can maintain guide bias for part of the pulse or control voltage transients. During active clamp or dynamic braking, the driver accelerates current decay. During an open-circuit or high-impedance state, the guide coil is substantially disconnected so that induced current in the unpowered winding is reduced. This distinction matters because a low-impedance closed or freewheel path can allow transformer-like induced current that opposes a changing magnetic flux and can load, damp, or alter the main solenoid pulse.

The Flux-Guide Coil 2255 is therefore not merely an added inductor. It is an actively controlled magnetic-path element. Its usefulness is evaluated by comparing distal B(t), radial divergence, pulse rise behavior, heat, and guide temperature with the guide coil open, passively present, pre-biased, concurrently energized, freewheeling, reset, and disabled. The controller can select among those states according to treatment mode, thermal headroom, desired distal emphasis, polarity sequence, and whether the applicator is operating as a full Operative Solenoid or in a directional flux-control mode.

### Distal Flux Lens

FIG. 8 further illustrates flux lens 610 at the distal end. The lens is a high-permeability field-shaping structure located distally with respect to the treatment chamber and around or near the distal opening of treatment zone 120. Its job is to improve the boundary condition at the end of the solenoid. Without the lens, field lines near the distal sidewall can turn outward early because air is available in every direction. With the lens, the distal perimeter offers a lower-reluctance structure that biases the field toward a more organized distal exit.

The term lens is used because the structure reshapes field distribution, not because it refracts magnetic flux in the same way glass refracts light. In engineering terms, the flux lens is a distal pole-piece or yoke-like field shaper. It can be a ring, an oval frame, a rectangular frame, a segmented perimeter member, a frustum, a set of individual plates, or another high-permeability member arranged at the distal perimeter.

The lens preferably does not block the treatment chamber. The purpose is not to replace the air-core treatment region with iron. The purpose is to improve the distal perimeter so that useful flux remains present through the distal digital end. The lens can surround the opening fully or partially, depending on the desired field and the need to avoid excessive shunting. For a foot chamber, an incomplete or segmented lens can be used to accommodate anatomy, housing geometry, thermal expansion, cleaning, or manufacturing.

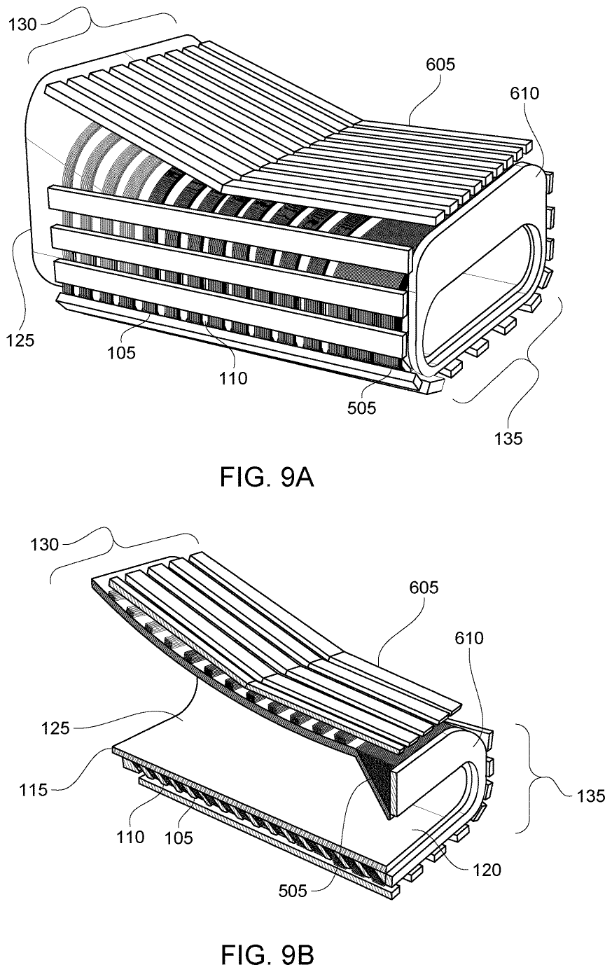

A flux lens is particularly useful when paired with distal turn-density shaping. The increased distal ampere-turns provide stronger local drive. The lens then reduces premature radial sidewall escape from that stronger distal field. Together they move the applicator from a simple finite solenoid toward a deliberately shaped distal magnetic circuit. FIGS. 9A and 9B illustrate, in perspective and sectional perspective respectively, an applicator combining exterior axial flux guides 605, a distal flux lens 610, and increased distal longitudinal turn density (LICD) 505, before addition of a distal flux target.

### Distal Flux Target

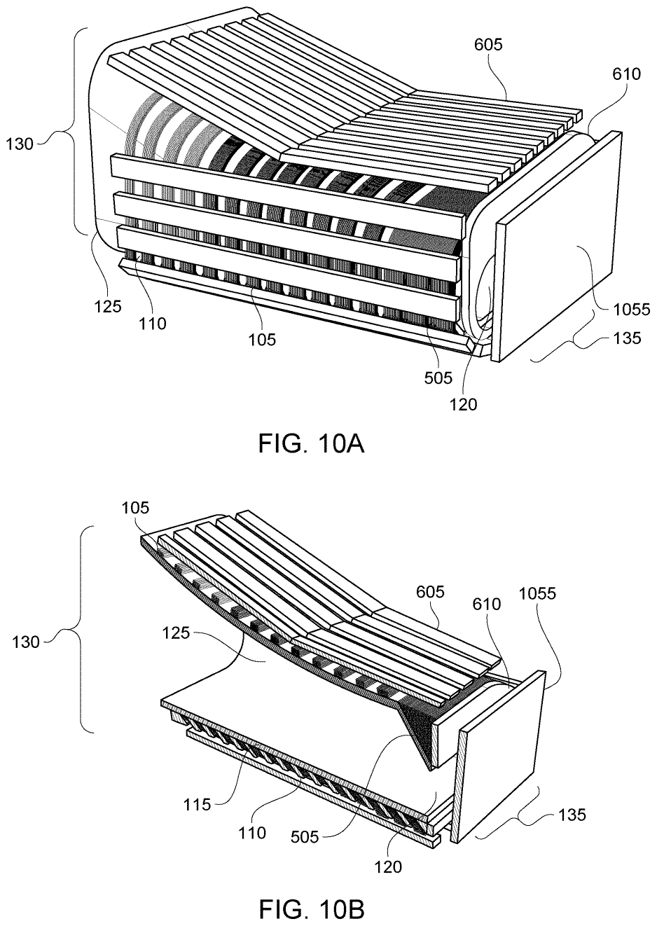

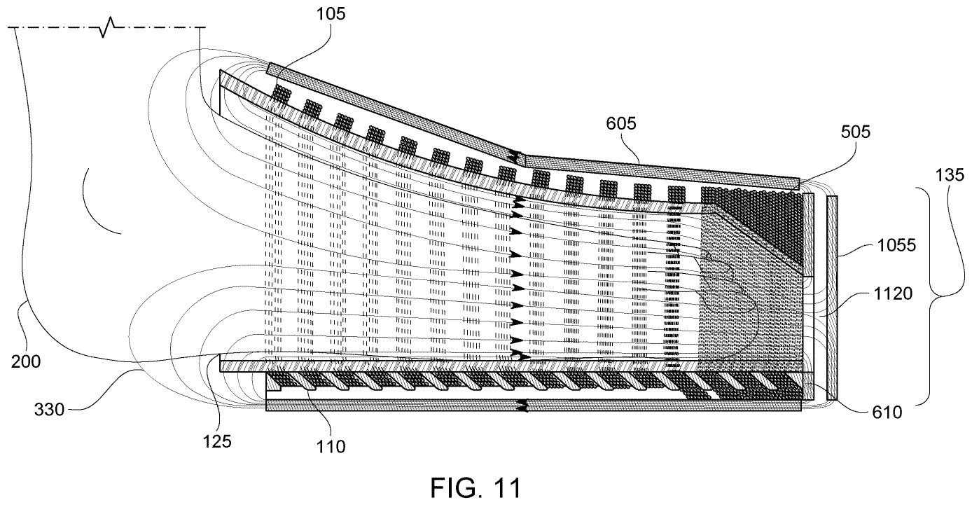

FIGS. 10A, 10B, and FIG. 11 add flux target plate 1055 beyond or adjacent to distal end 135. The target is a high-permeability destination for flux emerging from the distal region. When a flux lens is present, the target is preferably positioned distally of the flux lens so that the lens shapes the distal aperture and the target receives flux beyond that aperture. In the absence of a target, flux leaving the distal end and distal sidewall spreads into air and begins its uncontrolled return. With a target, the flux sees a lower-reluctance body at the distal end and is drawn toward that body. This tends to pull useful Core Flux farther through the distal digital region and reduces the amount of distal field that leaves prematurely through the sidewall before it returns externally.

The target works best when it is not merely a thin cosmetic sheet. A low-carbon steel target about 3 mm thick is a good practical thickness in representative compact embodiments, although other thicknesses can be used. The target should have enough cross-sectional area to conduct the intercepted flux without localized saturation. The target also benefits from a geometry that spreads flux into the axial flux guides or integrated guide-target member. A target that is too small or too narrow can saturate locally at the point where the flux enters the return path, especially near bends or corners.

The distal target gap 1120 between the distal opening and the target is functional. If the target is too far away, coupling is weak and the target has little influence. If the target is too close or too large, it can shunt flux into itself before the field usefully penetrates the distal treatment tissue. Representative embodiments use a gap selected by measurement, with gaps of about 1 mm to about 15 mm being useful in compact designs, and gaps of about 3 mm to about 5 mm being effective in some prototypes.

The target and lens perform different but related functions. The lens shapes the field at the distal perimeter and discourages sidewall escape. The target provides a destination beyond the distal end and encourages longitudinal continuation through the end of the chamber. In a strong embodiment, the lens and target are both present: the lens keeps the field organized near the aperture, and the target draws the field through that aperture.

### U-Shaped Guide-Target Member

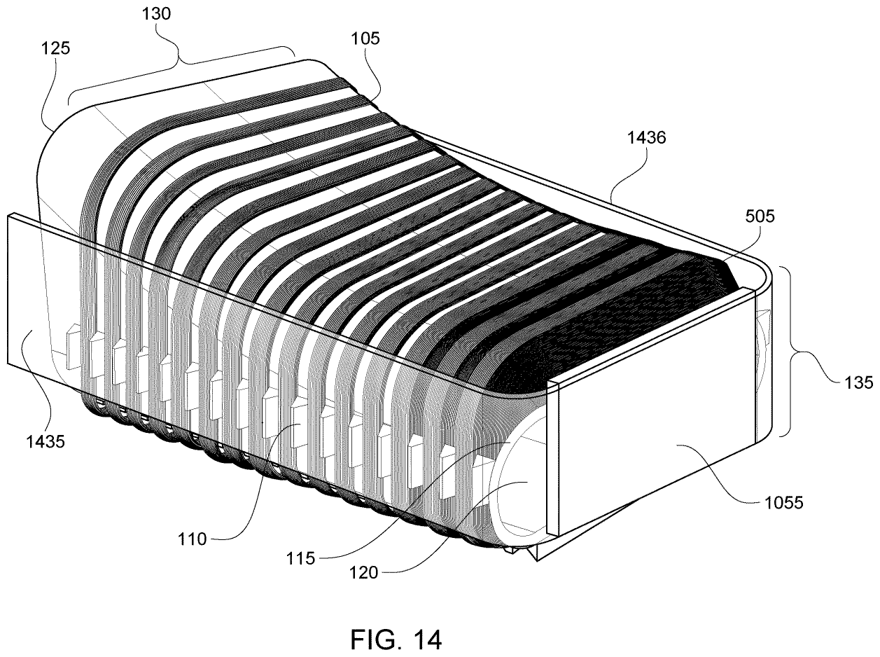

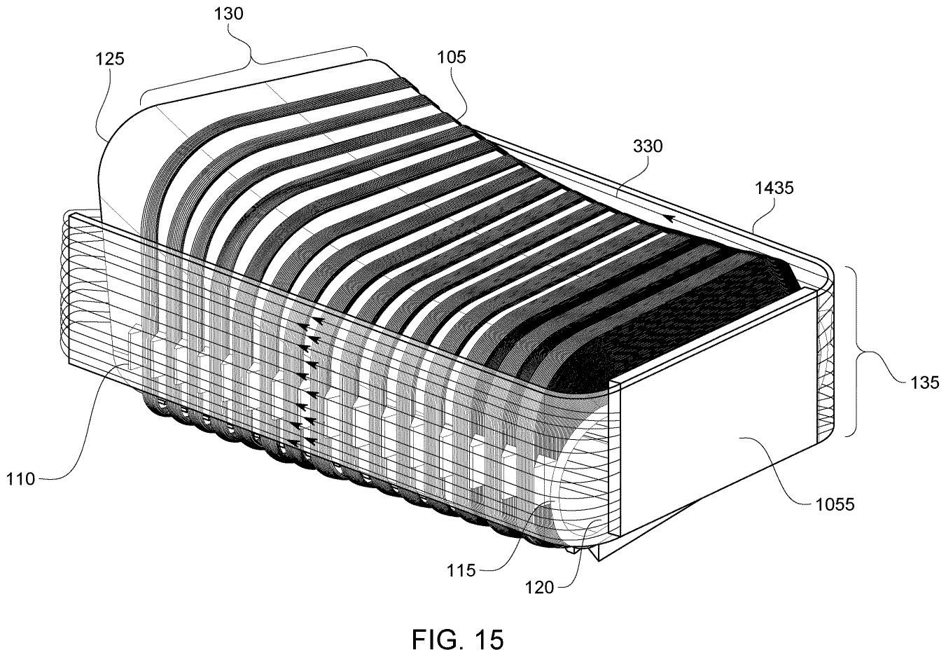

FIGS. 14 through 16 depict a U-shaped guide-target member 1435. A high-permeability strip, film, tape, or plate travels along one exterior side of the applicator, bends around the distal end, and returns along the opposite side. In the illustrated arrangement of FIG. 14, the U-shaped guide-target structure includes a lower guide strip portion 1435, an upper guide strip portion 1436, and a distal flux target plate 1055 positioned at the distal end; FIG. 16 shows the U-shaped guide-target member 1435 as a one-piece bent member exploded away from the distal target plate 1055. The distal bend or end region functions as a target, and the side portions function as axial flux guides. This creates a continuous magnetic return structure around the distal end.

The U-shaped member must be designed with saturation and eddy currents in mind. Pure iron can be one of the best materials magnetically in some low-field comparisons, but the additional performance often does not justify the extra cost in production when low-carbon steel performs adequately. A thin pure-iron tape has high permeability at low field but can saturate at bends or narrowed regions because the flux is concentrated into a small cross-section. A wider or thicker target portion at the distal end spreads the flux before it enters the side portions. Slots or laminations can also reduce eddy currents when pulse rise time is fast.

In some embodiments, the U-shaped member is formed as a flexible or semi-flexible strip retained by the housing. In other embodiments, it is stamped or bent from steel sheet. In still other embodiments, it is assembled from separate pieces joined magnetically or mechanically near the distal end. A small physical joint does not defeat the function if the joint provides adequate magnetic coupling for the intended pulse.

### L-Shaped Guide-Target Member

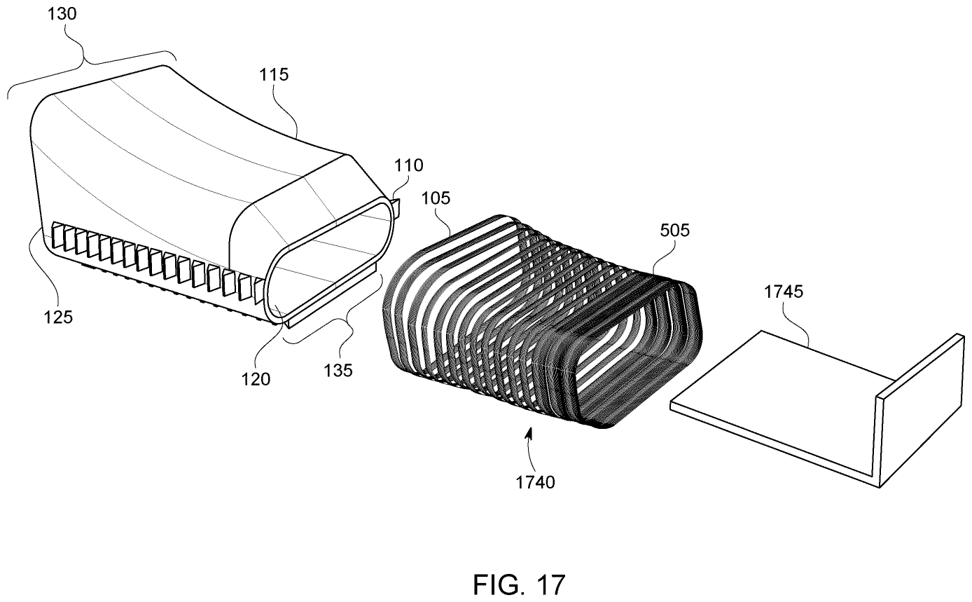

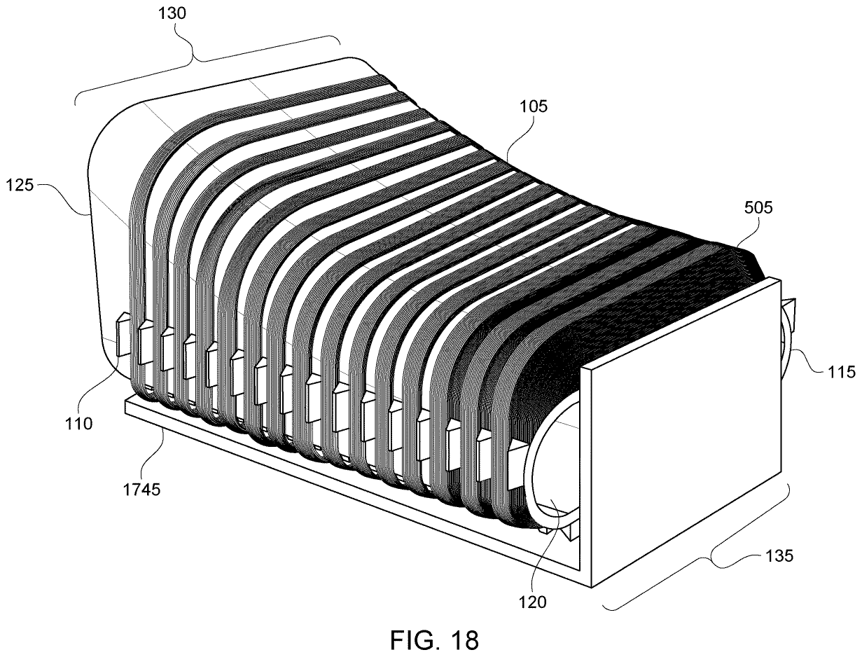

FIGS. 17 through 20 depict a preferred practical embodiment using an L-shaped flux guide and target 1745. The member includes a base plate extending along the underside or exterior side of solenoid 1740 and an upright end plate extending upward near distal end 135. The upright plate functions as the distal flux target and, when a flux lens is present, is positioned distally of that flux lens. The base plate functions as the axial return yoke. Together they form a simple, low-cost, high-impact magnetic-circuit member.

The L-shaped member is particularly attractive for manufacturing. In one representative implementation, the member is made from AISI 1018 low-carbon steel flat stock about 3 mm thick and about 50 mm wide. The stock is bent approximately 90 degrees to form the upright end plate. Other thicknesses, widths, materials, and bend geometries can be used. Useful materials include low-carbon steel, pure iron, silicon steel, ferrite, nickel-iron alloy, laminated steel, and powdered or composite magnetic material.

The L-shaped member is preferably spatially separated from Coil conductors and positioned by molded housing features, posts, channels, tabs, adhesive, overmolding, fasteners, or a combination of these. It does not need to carry electrical current. Its function is magnetic.

Cold forming is preferred for simple low-carbon steel implementations because it avoids unnecessary variation in magnetic properties from heating, cooling, oxidation, scale formation, or stress change. The disclosure should not be read as requiring a sharp bend. A radiused bend, segmented bend, welded joint, bolted joint, or assembled plate-and-base structure can also perform the function. The important point is magnetic continuity and adequate cross-section through the bend region.

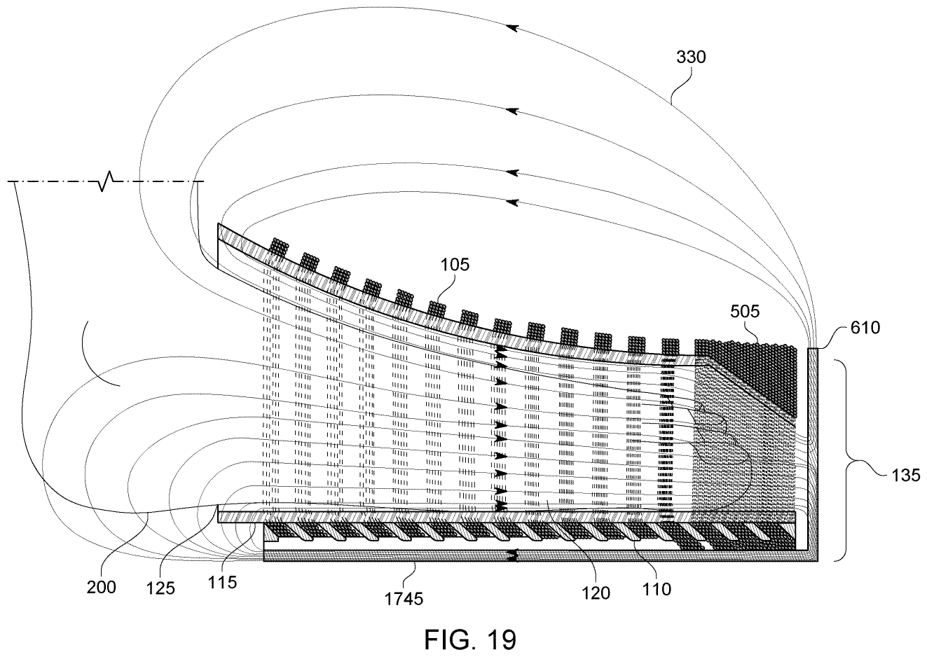

FIG. 19 shows the L-shaped member beneath the applicator and rising at the distal end near flux lens 610. The foot profile 200 enters through entry point 125 and advances toward treatment zone 120. The upright distal plate is positioned so that distal Core Flux is encouraged to continue through the distal digital region before entering the high-permeability member. The base then provides the return path toward the proximal region.

FIG. 20 shows the same concept from above. The foot enters through entry point 125, and the L-shaped member lies beneath the applicator and bends upward at distal end 135. When combined with LICD 505 and flux lens 610, it provides an integrated distal flux enhancement that is mechanically simple enough for production and magnetically meaningful enough to support multiple structural combinations.

FIG. 22 shows an embodiment in which a Flux-Guide Coil 2255 is positioned on or around an axial flux guide 605, illustrated as a bent plate-style guide member. In this arrangement, the guide coil can energize the base portion, the bent plate portion, or a transition region of the guide structure, and the same construction can be applied to an L-shaped guide-target member 1745 or other guide-target member. The coil remains external to the treatment chamber and can be insulated from the guide-target member by a bobbin, coating, tape, polymer sleeve, potting material, or molded housing feature.

### Temperature-Responsive Magnetic-Flux Optimization, Magnetic Flux Density Curves, and Thermal Headroom

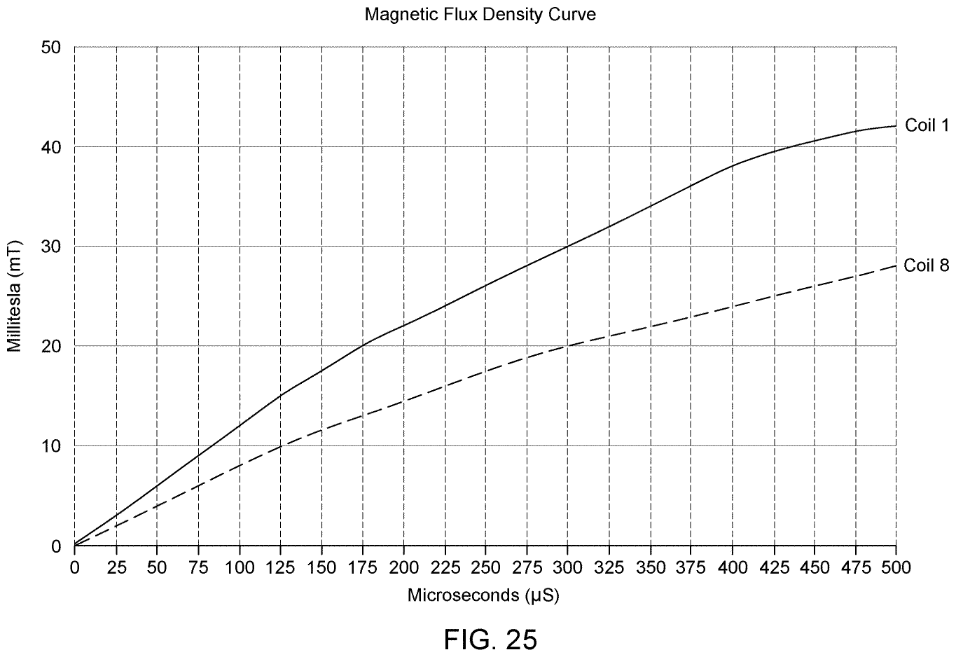

FIG. 25 depicts a Magnetic Flux Density Curve for representative Coils, with Magnetic Flux Density on the y-axis in millitesla and time on the x-axis in microseconds. In the illustrated example, the solid curve represents Coil 1 and the dashed curve represents Coil 8. The curve illustrates why a single static flux number is incomplete for a pulsed MPTD. An inductive Coil does not produce its final field instantly. Magnetic Flux Density rises as current rises, and the useful treatment quantity can depend on peak B, B at a specified time, dB/dt, pulse width, pulse polarity, repetition rate, and the thermal state of the device.

For example, a representative Coil can reach about 20 mT after one pulse duration and a higher Magnetic Flux Density after a longer pulse duration. A longer pulse can therefore increase distal-end field strength without changing the hardware. The cost is heat and, in some conditions, reduced incremental dB/dt as the Coil current approaches its practical limit. Pulse duration is therefore a control variable, not merely a therapy setting.

A controller can store or determine a Magnetic Flux Density Curve for each relevant Coil or Coil group. The curve can be programmed from manufacturing calibration data, measured during production, derived from a fixture measurement, or updated using diagnostic routines. A preferred calibration method uses an AlphaLab MagWave B-Probe connected to a digital oscilloscope to capture B(t) for representative pulse durations and Coil combinations.

The controller can also store or determine guide-coil timing and current data for a Flux-Guide Coil 2255. A guide-coil control table or curve can specify pre-bias duration, guide-coil current, polarity, overlap with treatment Coil energization, freewheel duration, clamp timing, reset polarity, and thermal limits. The guide-coil command need not have the same pulse width or waveform as the treatment Coils because its function is to control the external magnetic return path rather than directly encircle the treatment zone.

More generally, the controller can store or access model data representing relationships among pulse timing, pulse duration, pulse repetition frequency, rest interval, pulse waveform, selection of Windings, Winding groups, taps, Coils, or Coil segments to be energized, forced-return enablement at the end of a pulse, Flux-Guide Coil operating states, Magnetic Flux Density delivered in the treatment chamber, and heat generation in the applicator. The model data can be a lookup table, family of calibrated B(t) curves, heat-rise model, thermal time-constant model, empirical map, finite-element-derived model, production calibration dataset, or an adaptive model updated during use.

The controller can then select one or more pulse parameters based on objective conditions. When the device is cool, the controller can use longer distal pulses, higher pulse frequency, shorter rest intervals, selected distal Coil activation, selected pulse waveform, selected forced-return behavior, or occasional longer distal pulses to increase distal-end Magnetic Flux Density. As temperature rises, it can shorten pulse duration, reduce repetition rate, change Coil selection, change waveform, alter a forced-return state, change a Flux-Guide Coil state, increase rest intervals, reduce intensity, or terminate treatment if the remaining dose cannot be delivered within the available thermal envelope.

Temperature-responsive magnetic-flux optimization differs from a simple cutoff thermostat. A thermostat merely stops operation or changes state after a temperature threshold is reached. In the disclosed MPTD, temperature data is used affirmatively to determine available thermal budget and to select or update pulse parameters so that therapeutic magnetic flux delivered to the treatment chamber is increased, preserved, or delivered more efficiently within the available thermal operating margin.

In a tapered solenoid applicator, the distal winding region often runs hottest because the distal region may include increased winding density, tighter packing, poorer local convection, and distal flux-enhancement structures that concentrate useful magnetic activity. That distal temperature can become the limiting factor for the whole protocol. A fixed protocol must be safe when the applicator starts at a high expected ambient temperature and therefore must assume a reduced thermal budget. In representative products, this conservative derating can reduce deliverable magnetic-flux dose by about 20% to about 25%, and in some cases more, compared with a protocol that uses the actual measured starting temperature and real-time temperature history.

The thermal budget can be determined from a temperature limit, the measured starting temperature, later temperature data, expected heat generation, heat capacity, cooling rate, ambient conditions, elapsed treatment time, and the desired dose. Because the upper temperature is fixed by safety, comfort, consumer-product requirements, or medical-equipment requirements, a cooler starting Coil temperature leaves more thermal headroom. The controller can therefore use the measured starting temperature and later temperature readings to select a higher total dose, a shorter treatment time, a higher session throughput, or a combination of those outcomes while still maintaining the sensed temperature at or below the limit.

Parameters selected or updated by the controller can include pulse timing, pulse duration, pulse repetition frequency, rest interval, pulse waveform, energized Winding selection, energized Winding-group selection, tap selection, energized Coil or Coil-segment selection, forced-return enablement at the end of a pulse, active discharge, active clamping, polarity reversal, and the operating state of any Flux-Guide Coil. The controller can follow a precomputed plan, adapt the plan during treatment using real-time temperature data, or combine a stored plan with dynamic correction. In each case, the feature is used to optimize magnetic-flux delivery, target magnetic-flux dosage, treatment time, or session availability within a thermal budget, rather than merely to shut the device off after overheating occurs.

Not every pulse needs to be lengthened. For a device operating at a repetition rate such as 50 pulses per second, a smaller number of longer pulses per second can be assigned to distal Coils while other pulses remain at the normal duration. This allows the device to deliver enhanced distal treatment without converting the entire session into a high-heat operating mode. The same concept can be used with DDFC or other sequencing modes.

Not every operating mode benefits from active guide energization. In full-solenoid modes, a Flux-Guide Coil 2255 can be used to assist longitudinal return flux and increase distal-end field organization. In modes that intentionally redirect flux to variable points of opposition, the same guide coil can be left open, disabled, reset, reversed, or timed differently so that it does not impose an unwanted axial return path.

Thermal control is especially important because the user may have reduced temperature sensation in the foot. The device should monitor temperatures that represent the hottest or most safety-relevant locations. Distal-mounted LM75-type sensors thermally coupled to distal Coils or adjacent winding-retention structures are useful in production when embedded sensors are included. Thermal imaging is preferred during development because it reveals whether the assumed sensor locations correspond to actual hot spots.

### Material Selection, Saturation, and Eddy Currents

A useful guide, lens, or target is selected for the pulse environment, not for a catalog permeability number alone. Low-carbon steel such as AISI 1018 has proven to be a good choice because it is low cost and performs adequately for many MPTD flux-management functions. Pure iron can have higher low-field permeability but can be more expensive and may offer little practical benefit if the geometry, air gaps, or saturation dominate performance.

Ferrite offers high resistivity and therefore lower eddy-current loss, but ferrite is brittle and often has lower saturation flux density than steel. Silicon steel laminations can be excellent when eddy currents matter, but they add manufacturing complexity. Nickel-iron alloys can offer high permeability but can be expensive and sensitive to stress or heat treatment. The disclosed invention does not depend on one exact material; it depends on a high-permeability path that remains effective under the intended pulses.

Saturation must be evaluated in the magnetic member itself, not merely in the free-space treatment field. A treatment chamber field of tens of millitesla can correspond to a much higher flux density inside a narrow guide because the guide concentrates flux into a smaller cross-sectional area. Bends, corners, holes, and narrow necks are common saturation locations. Increasing cross-section at these regions or using a target plate to distribute flux before it enters a guide reduces localized saturation.

Eddy currents are also geometry-dependent. A continuous conductive plate exposed to rapidly changing perpendicular flux can support circulating currents. Those currents oppose the field change and generate heat. In sidewall guide structures, keeping individual guide widths in about the 7.5 mm to 15 mm range, and preferably 15 mm or less, reduces conductive loop area and keeps eddy currents minimal in representative pulse designs. For that reason, two or more spaced sidewall flux guides are often better than one large sidewall plate, even when the total magnetic cross-section is similar. Slots, gaps, laminations, ferrite, powdered magnetic materials, or orientation changes can also reduce eddy-current effects. In a compact MPTD, the design choice depends on whether measured B(t) and thermal rise indicate that eddy currents are materially affecting performance.

Active guide energization introduces an additional saturation variable. The magnetic operating point of a guide or guide-target member depends on the solenoidal field, the Flux-Guide Coil 2255, remanent magnetization, air gaps, material B-H behavior, and prior pulse history. A small bias can be useful if it places the guide in a favorable incremental-permeability region or provides cooperating magnetomotive force, but a large bias can reduce incremental permeability by moving the material toward saturation. The controller can therefore limit guide-coil ampere-turns, alternate polarity, apply a reset pulse, or place the guide coil in a high-impedance state between pulses.

The preferred engineering workflow is therefore empirical as well as analytical. A candidate guide-target geometry is built, the distal field is measured with a fast magnetic probe and oscilloscope, thermal images are taken under representative pulse protocols, and the member is adjusted for cross-section, gap, slotting, or placement. This is the fastest way to determine whether a particular member is guiding flux or merely adding mass.

### Measurement and Validation of Distal-End Improvement

The improvement produced by the disclosed structures is measurable. A baseline applicator is first measured without the distal-end enhancement being tested. The enhanced applicator is then measured under the same-test energization condition. The measurement fixture preferably holds the sensor at repeatable positions in the treatment chamber and at repeatable distal positions near the distal digital region, the distal opening, and just beyond the distal opening.

For distal-end evaluation, useful measurements include longitudinal B(t) along the treatment chamber, radial B(t) from the chamber toward the sidewall, peak B at the distal treatment location, B at a specified elapsed time such as 175 microseconds, 300 microseconds, or 500 microseconds, dB/dt during the rise interval, and time-integrated flux change. Comparing the longitudinal and radial components helps determine whether the structure has actually improved distal coherence or merely increased a local stray field.

The preferred magnetic measurement system is an AlphaLab MagWave fast magnetic waveform sensor, including a MagWave B-Probe, connected to a digital oscilloscope. The oscilloscope should record enough bandwidth and sample rate to capture the relevant rise time and pulse shape. The probe axis should be identified. If a three-axis method is used, the longitudinal, radial, and transverse components should be recorded or converted into a defined coordinate system.

Alternative measurement methods include Hall-effect sensors, search coils, calibrated gaussmeter probes, three-axis magnetometers, magnetic-field cameras, and numerical field models validated by physical measurement. These alternatives are acceptable when the pulse bandwidth, amplitude range, spatial resolution, and timing accuracy are sufficient. A slow sensor that reports only an averaged field is not suitable for validating microsecond pulse behavior.

A practical validation sequence is as follows. The baseline solenoid is measured along a longitudinal path from the proximal region through the distal digital region. The same path is measured with axial flux guides installed. The same path is then measured with the flux lens, the flux target, and the integrated guide-target member in selected combinations. The point is not to prove that every feature is always required. The point is to determine how each feature changes B(t), radial divergence, and distal-end field organization.

When a Flux-Guide Coil 2255 is included, validation preferably compares several electrical states of the guide coil, including open circuit, passive unenergized presence, pre-bias, concurrent energization, post-pulse reset, freewheel or recirculation, and active clamp. These measurements determine whether the guide coil improves treatment-region B(t) or merely adds coupled inductance, heat, or damping. The comparison is preferably made under the same-test energization condition used for the corresponding treatment Coil or Coil group.

Thermal validation is performed under representative or worst-case pulse protocols. A thermal imaging camera is used to identify hot spots on the distal Coils, guide-target members, housing surfaces, and user-facing chamber surfaces. If embedded LM75-type sensors are present, their readings are logged and correlated to thermal images. The controller can then use the embedded readings with confidence because the sensor locations have been correlated to actual hot regions.

### Interaction With Dynamic Directional Flux Control

Dynamic Directional Flux Control, or DDFC, is disclosed in the related MPTD family. DDFC changes field orientation by energizing selected solenoid segments with controlled polarity relationships and leaving or creating gaps through which flux can be redirected. The present invention is not primarily a DDFC invention. It is a distal-end flux-enhancement invention. Nevertheless, the two technologies interact beneficially when the active guides are controlled for the selected mode rather than treated as always-on structures.

DDFC works better when sufficient flux is available in the region being redirected. Near the distal end of an unenhanced solenoid, the available field is weaker and less organized because of end effects. The distal flux enhancements disclosed here increase the usable magnetic field near the distal digital region. That improved field can then be redirected by DDFC modes more effectively than a weak, already-diverging field.

The enhancement structures also give DDFC a better magnetic environment near the distal aperture. A flux lens can reduce uncontrolled sidewall escape. A flux target can draw field through the end. Axial guides can provide a defined return path. A Flux-Guide Coil can further control the guide state. However, DDFC can intentionally create opposing solenoidal regions rather than a single distal-to-proximal return path. At a DDFC opposition point, the distal-facing and proximal-facing ends of the opposing solenoids can present the same magnetic polarity. In that condition, powering a longitudinal guide may offer little or no benefit and can be neutral or counterproductive. If the applicator is intended to operate exclusively in such DDFC modes, powered flux guides may be omitted or normally disabled. Passive, unpowered flux guides can still be useful with DDFC. Flux that leaks through the sidewall at or near the opposition point is attracted to the high-permeability guide, and the guide can conduct the collected flux proximally and distally away from the opposition point, reducing uncontrolled sidewall return paths without imposing a powered longitudinal guide field. The controller can therefore enable, disable, open-circuit, freewheel, reverse, or reset the Flux-Guide Coil according to the selected DDFC pattern.

Commingled coil-segment Windings can improve the magnetic environment in which DDFC operates because they reduce hard inter-segment discontinuities while preserving the separately energizable segment structure used by the controller. Thus, a DDFC pulse pattern can still select adjacent or spaced coil segments, reverse polarity in selected segments, and leave one or more intermediate segments unpowered, while the commingled winding geometry reduces unnecessary local field aberrations at physical segment boundaries.

### Representative Construction Examples

One representative applicator uses a plurality of Coils 105 arranged around treatment chamber 225, with each Coil separated by a separator 110. The Coils use copper wire in the range of about 18 AWG to about 24 AWG and are driven from a low-voltage driver, such as a 24 VDC driver using half-bridge switching. The exact wire size, number of Turns, and Coil length are selected to produce the desired pulse rise behavior and thermal performance.

In a preferred tapered-core embodiment, the core structure includes molded winding-retention features 110 aligned with intended coil divisions. The features 110 can include dividers, wire retainers, sidewall barbs, lower channels, lead-routing pockets, strain-relief structures, or other plastic structures that guide start and finish conductors and hold the wound coil mass. These features allow adjacent coil segments to commingle where magnetically useful while preventing uncontrolled winding migration or conductor loosening.

In a preferred eleven-coil embodiment, each coil segment is spin wound sequentially from distal to proximal on the tapered chamber core using about 20 AWG insulated conductor. Other embodiments use different wire gauges, coil counts, or winding sequences. The core can be rotated on a shaped spindle while the supply wire is tensioned and guided. Because the taper and retention structures cause the Windings to pack and lock against each other, the process can be faster and more repeatable than winding isolated bobbins and subsequently stacking or assembling them onto an irregular chamber core.

A representative distal turn-density enhancement uses shorter or overlapping distal Coils near distal end 135. The distal Coils have increased conductor density per unit longitudinal length relative to more proximal Coils. The increase is selected to improve distal Magnetic Flux Density while preserving acceptable dB/dt and heat generation. The increase can be continuous, stepped, triangular, layered, or localized.

A representative axial guide set uses low-carbon steel bars or plates extending along at least part of the applicator exterior. Each guide has a proximal region and a distal region. The guides are positioned outside the Coil Windings and outside the treatment chamber, with sufficient insulation and spacing to prevent electrical contact with the Coils. The guide cross-section is selected so that the guide remains below substantial saturation during the intended pulse.

A representative active guide embodiment uses one or more Flux-Guide Coils 2255 wound around or otherwise coupled to a selected axial guide, pair of axial guides, plate guide, or L-shaped guide-target member. The Flux-Guide Coil can use insulated copper wire selected for the expected duty cycle and can be driven by a microprocessor-controlled branch driver from the same nominal 24 VDC supply used for the treatment Coils or from a different guide-coil supply. The driver preferably supports at least two states, active drive and high impedance, and can additionally support bidirectional polarity, current regulation, freewheel, active clamp, or reset.

A representative flux lens uses a high-permeability frame around the distal opening. The frame can be a continuous ring-like member or a plurality of separate plates arranged around the distal aperture. A spacing of a few millimeters from adjacent Coil structures is useful in some constructions, but the final gap is selected by measurement.

A representative flux target uses a low-carbon steel plate positioned beyond distal end 135 with a gap from the treatment chamber. In compact embodiments, the plate can be about 1 mm to about 10 mm thick, with about 3 mm to about 5 mm being practical for low-carbon steel. The plate area is large enough to intercept the desired distal flux and couple it to axial return guides without excessive saturation.

A preferred integrated guide-target member is an L-shaped low-carbon steel member. The base plate lies under the applicator and extends from the distal region toward the proximal region. The upright plate stands near the distal end and functions as the target. A representative member is formed from AISI 1018 steel flat stock about 3 mm thick and about 50 mm wide. Other dimensions are used when the treatment chamber, Coil geometry, pulse width, and measured field require them.

In a preferred active-guide construction, the Flux-Guide Coil 2255 is placed on the base plate of the L-shaped member or around two side bar guides because those locations provide useful coupling to the external return path without occupying the treatment chamber. The guide-coil Windings are insulated from the ferromagnetic member and from the treatment Coil conductors. The housing can include channels, ribs, bobbin features, or overmolded portions that set the guide-coil position and prevent motion during repeated applicator use.

The L-shaped member can be retained by the housing without separate fasteners if the housing includes molded channels, ribs, stops, posts, or clamping surfaces. In another construction, the member is bonded, screwed, riveted, overmolded, potted, or captured between housing components. The member is preferably positioned before the Coil-supporting core is inserted into the housing, so assembly is repeatable.

### Production Dual-Foot Embodiment and Control Hardware

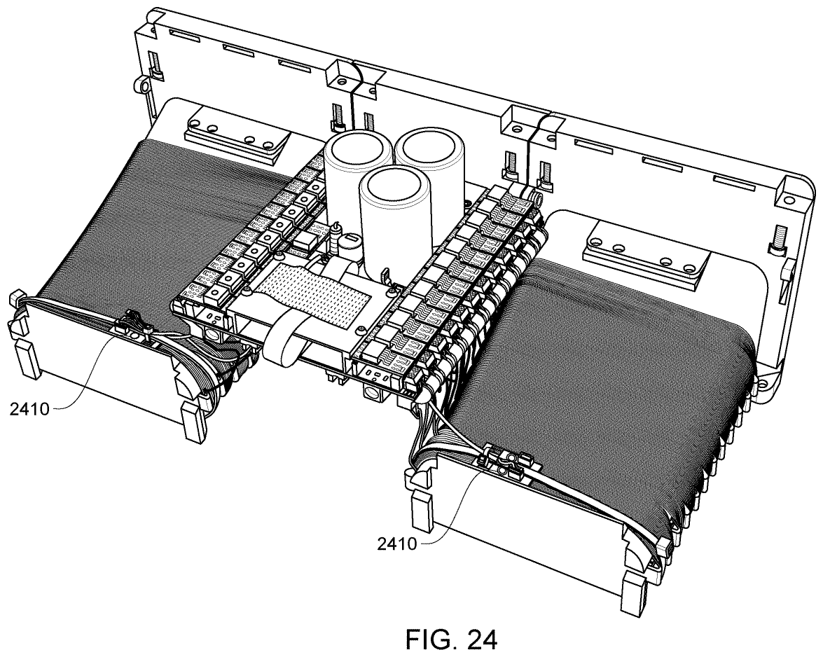

FIG. 24 illustrates a production-oriented dual-foot, dual-chamber MPTD applicator as viewed from the distal end with internal components exposed. The applicator includes a left treatment chamber and applicator core assembly and a right treatment chamber and applicator core assembly, each carrying a solenoid winding assembly disposed around the respective chamber assembly.

The production embodiment of FIG. 24 also illustrates a central electronics region positioned between or near the two chamber assemblies. Driver boards, branch drivers, switching circuits, or driver arrays can be located in the central electronics region and can energize selected coil segments of the left and right winding assemblies. The drivers can also energize Flux-Guide Coils when active guide energization is included.

In one production embodiment, the pulse-energy supply includes three energy-storage capacitors, each having a capacitance of about 15,000 microfarads, visible in the central electronics region of FIG. 24. The capacitors can be coupled to the low-voltage driver circuitry to support high-current magnetic pulses without requiring a high-voltage clinical-style pulse source. Other numbers, values, and arrangements of capacitors can be used according to pulse energy, recharge time, thermal limits, cost, and packaging.

The embodiment of FIG. 24 can include a left LM75-type digital temperature sensor 2410 thermally coupled to the left winding assembly or an adjacent thermally representative structure, and a right LM75-type digital temperature sensor 2410 thermally coupled to the right winding assembly or an adjacent thermally representative structure. Separate left and right temperature sensing allows the controller to determine chamber-specific thermal budgets and to increase, maintain, redistribute, or reduce magnetic-flux delivery by changing pulse duration, pulse repetition rate, rest interval, energized segment selection, waveform, forced-return state, guide-coil state, or treatment termination independently or globally if either chamber approaches a thermal limit.

FIG. 24 also illustrates production wire-management channels, molded guides, harness-routing structures, clips, or strain-relief structures. These structures route conductor leads from the wound coil segments to the drivers, keep leads from crossing user-facing chamber surfaces, reduce conductor motion during handling, and reduce the chance that a wound segment will loosen, unfurl, or be displaced before final housing closure. Additional winding retention or guide structures can cooperate with features 110 on the chamber core.

Prototype or development views, including FIG. 20 and similar assembled winding views, may omit some production wire-management channels, molded guides, routing features, and retention structures for clarity or because they illustrate hand-built prototypes. FIG. 24 shows how the same flux-enhancement and commingled-winding principles can be implemented in a production dual-foot assembly with driver electronics, capacitor-supported pulse delivery, temperature sensing 2410, and integrated wire management.

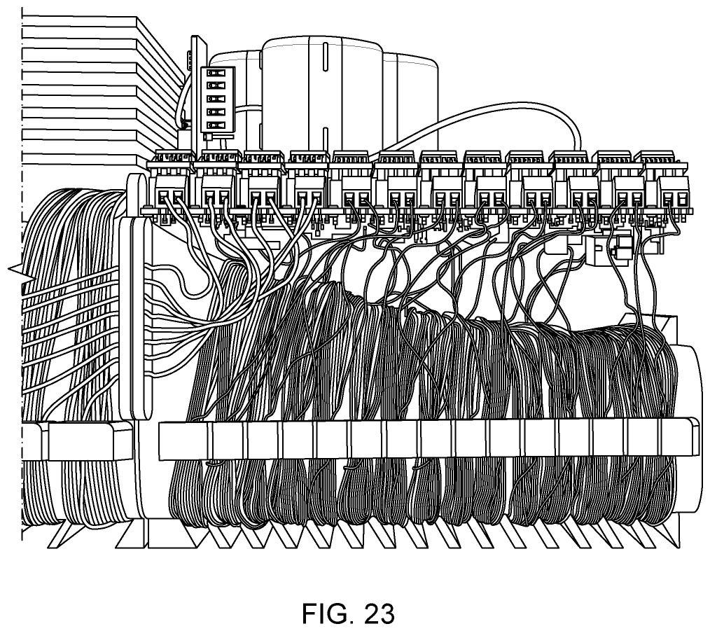

FIG. 23 illustrates an internal side view of a segmented solenoid applicator, showing electromagnetic coil-wiring routing and wire-management features within the rigid housing structure and coil supports. A rigid housing and coil-support structure supports the wound coil segments and defines datum surfaces, channels, and standoffs that position the winding assembly. Electromagnetic coil wiring, including the lead conductors that connect each separately energizable coil segment to its driver, is routed along the interior of the housing structure by wire-management features. The wire-management features can include molded guides, harness channels, clips, lead-routing pockets, strain-relief structures, and comparable features that keep the coil-segment leads off the user-facing chamber surfaces, reduce conductor motion during handling and pulsing, and maintain repeatable lead placement from unit to unit. The wire-management features cooperate with the winding-retention features 110 on the applicator core structure so that the coil segments remain seated and separately energizable after assembly.

### Alternative Embodiments and Design Freedom

The disclosed structures are modular. An embodiment can use solenoid elongation without a flux target. Another embodiment can use a flux lens and target without separate axial guide bars because an integrated L-shaped member supplies the return path. Another embodiment can use axial guides alone to improve external return flux. These structural combinations are independently useful and can be implemented according to the desired product configuration.

The treatment chamber can be sized for a whole foot, forefoot, both forefeet, a hand, or another appendage. The distal-end problem is most visible in a distal digital-region foot applicator, but the same field behavior occurs whenever the treatment target lies near the end of a finite solenoid. A hand embodiment, for example, can use a distal target near the fingertips.

The high-permeability structures can be continuous or discontinuous. They can be symmetric or asymmetric. Symmetric structures tend to preserve a balanced field. Asymmetric structures can intentionally bias field toward the plantar surface, dorsum, medial side, lateral side, or another anatomical region. Asymmetry can be useful when target nerves are closer to one chamber surface.

The guide-target member can be L-shaped, U-shaped, C-shaped, J-shaped, plate-like, frame-like, segmented, laminated, slotted, or assembled from multiple pieces. The term guide-target member describes function, not a single geometry. What matters is that the member both influences distal flux and provides or couples to a return path. When active guide energization is used, a Flux-Guide Coil can be placed on any magnetically useful portion of that member or omitted from portions where active excitation is not helpful.

The controller can operate all distal Coils with the same pulse duration, or it can use longer pulse durations only in selected distal Coils. It can use longer pulses only at the beginning of a session, only after confirming available thermal headroom, or only in a subprotocol intended to emphasize distal digital-region treatment. It can also use shorter high-dB/dt pulses when the therapy objective favors rise rate rather than peak B.

The controller can also account for manufacturing variation. If calibration shows that a particular applicator has stronger or weaker distal response, the stored Magnetic Flux Density Curve can be used to adjust pulse duration, Coil selection, or repetition rate. This allows the device to deliver more repeatable distal dosing despite variation in winding tension, material permeability, housing dimensions, and sensor placement.

The controller can also account for mode-dependent guide usefulness. A full-solenoid treatment mode can use active guide energization to assist distal-to-proximal return flux. A directional mode can disable or alter the Flux-Guide Coil so that the guide does not defeat a desired local opposition path. A calibration table can store different guide-coil timing, polarity, and driver-state commands for different treatment protocols.

### Technical Advantages

The disclosed distal-end architecture provides several technical advantages in the MPTD treatment chamber. It increases useful Magnetic Flux Density in the distal digital or distal treatment region, reduces premature radial divergence through the sidewall, gives returning flux a lower-reluctance path, permits active energization or timing control of selected flux guides, and improves the predictability of DDFC near the distal end when active guides are coordinated with the DDFC pattern. These are therapeutic-delivery advantages: they help the medical device deliver stronger and better organized magnetic pulses to distal foot tissue without relying on clinical high-power brute force.

The architecture also preserves the central advantage of the related segmented-solenoid MPTD: the appendage remains inside the core of the magnetic source. A high-permeability guide is not placed where the foot belongs. The foot still occupies the treatment chamber. The magnetic members modify the surrounding reluctance environment so that the chamber field becomes more useful.

The distal enhancements are especially favorable in low-voltage systems because low-voltage systems have limited excess field available to waste. A high-power clinical device can sometimes tolerate inefficient field geometry by overpowering the problem. A low-voltage home-suitable MPTD cannot. Improving magnetic efficiency and distal field organization is therefore not ornamental; it is central to obtaining useful distal treatment without turning the device into a high-power clinical machine.

### Why the Solenoid Architecture Makes the Enhancements Possible

The present distal-end structures are best understood as improvements to the solenoidal applicator of an MPTD, not as general magnetic accessories. A planar coil, puck, or pad does not have a longitudinal treatment core that terminates at a distal digital chamber end. It has a surface field that already spreads from the coil face and perimeter. A backing plate can recover field from an unused side of such a planar applicator, but that is a different medical-device problem. The present applicator begins with organized Core Flux inside a hollow treatment chamber and then corrects the finite-solenoid end effect at the chamber terminus.

Because the segmented solenoid produces a field that is already directionally organized, a distal target or lens can influence a large portion of the useful treatment field. In a randomly oriented non-core field, placing a plate near one edge can collect some local leakage but does not pull an organized longitudinal field through a treatment chamber. In the present design, the distal-end structures interact with a coherent field volume. This is why a relatively simple high-permeability member, properly placed, can measurably improve Magnetic Flux Density delivered to the distal digital region.

The solenoid also creates a natural distinction between useful flux and return flux. Useful flux is the flux traveling through the treatment chamber where the foot is located. Return flux is the flux outside the chamber completing the magnetic circuit. The disclosed structures improve both sides of that distinction. The lens and target help keep useful flux traveling through the distal chamber. The axial guides and base portion of an L-shaped member improve the return path after the flux has served its treatment function.

Active guide energization is also meaningful because the solenoid distinguishes treatment Core Flux from external return flux. A Flux-Guide Coil on an external guide does not replace the treatment Coils and does not occupy the chamber. Instead, it controls the magnetic state of the return-path structure that receives flux after the field has served the treatment region. This separation allows the guide to be energized, disabled, or reset without changing the basic appendage-receiving geometry.

### Longitudinal Coil Packing and Diminishing Returns

The segmented solenoid allows field strength to be increased by adding longitudinal segments, but only within practical limits. If Coils are placed closer and closer together, the device eventually reaches a point where additional conductor primarily increases mutual inductance, resistance, heat, driver cost, and assembly complexity rather than useful distal Magnetic Flux Density. The optimum is therefore not maximum packing density. It is the density at which the selected Coils cooperate as an Operative Solenoid while preserving the desired pulse rise time and thermal behavior.

For a low-voltage MPTD, this issue is significant. The driver has limited voltage available to force current through an inductive load. A longer pulse can allow current to rise further, but longer pulses consume thermal headroom. More Turns can increase magnetomotive force after the current rises, but they also increase inductance and slow that rise. Larger wire can reduce resistance and heating, but it occupies more space and can reduce the number of Turns that fit in the available geometry. The design is therefore a multi-variable tradeoff among ampere-turns, inductance, resistance, dB/dt, temperature rise, chamber size, and manufacturing cost.

Distal turn-density shaping is useful because it does not treat the entire solenoid as if every axial position has the same need. The central region of the chamber already benefits from neighboring Coils on both sides. The distal end does not. Adding conductor density or shortening Coil pitch near the distal region corrects a local weakness rather than globally increasing the burden on the entire device. This is an engineering-efficient use of added conductor.

The same logic applies to solenoid elongation. Adding a distal extension moves the end effect, but each added Coil consumes length, conductor, driver resources, and power budget. A compact foot applicator benefits from using just enough elongation to move the worst end effect away from the distal phalanges and then using magnetic-path enhancements to do the remaining work. This combined approach is more compact than a long solenoid and more efficient than simply increasing drive power.

### Field Sequence During an Enhanced Pulse

A single pulse through an enhanced applicator can be followed as a sequence. At pulse initiation, or before pulse initiation when active guide energization is used, the controller applies commanded drive states to the selected treatment Coils and any selected Flux-Guide Coil 2255. Coil current begins to rise according to the inductive load, resistance, driver limit, and any current-control strategy. The rising current produces magnetomotive force around the treatment chamber. Neighboring selected Coils magnetically couple, and Core Flux forms along the chamber axis.

In the central region of the chamber, the field resembles the useful field of the baseline segmented solenoid. In the distal region, however, the enhanced structures change what would otherwise happen. Distal turn-density shaping supplies additional local magnetomotive force. The distal flux lens presents a high-permeability perimeter condition that discourages early radial escape. The distal target presents a high-permeability destination beyond the distal aperture. If a Flux-Guide Coil has pre-biased or actively energized a guide, the external return path can participate earlier or more strongly during the same interval. The field therefore has a stronger tendency to continue through the distal treatment region before returning externally.

After the flux reaches the distal region and couples to the target or guide-target member, the return path becomes important. The flux does not terminate at the target. It must close its path. The axial guides or base portion of the L-shaped member provide a lower-reluctance route toward the proximal region. A Flux-Guide Coil 2255 can add controlled magnetomotive force to that route, maintain a selected guide bias during part of the pulse, or be placed in a high-impedance state when active guide participation is not desired. This return path reduces the need for the flux to spread widely through air. A more disciplined return path makes the treatment chamber field more predictable and reduces wasted external field.

As the pulse continues, the current and B(t) may rise further. If the pulse is short, the benefit is dominated by early dB/dt and the field at the specified elapsed time. If the pulse is longer, peak Magnetic Flux Density can increase, but heat and saturation become more important. If the guide-target member approaches saturation, its incremental benefit decreases. If eddy currents are significant, the member can delay or oppose part of the field change. These effects are why the preferred design uses both magnetic measurement and thermal measurement rather than relying only on static geometry.

When the pulse ends or reverses, the field collapses or changes polarity. The passive high-permeability structures remain useful during the opposite polarity portion of a bipolar pulse because magnetic reluctance is generally polarity-symmetric in the absence of permanent magnetization effects. A Flux-Guide Coil 2255 can be commanded to reverse polarity, freewheel, clamp, reset, demagnetize, or open depending on whether guide current should persist into the next pulse. Hysteresis, eddy current, saturation, and coupled-winding behavior should be verified for the selected material and pulse protocol.

### Tuning the Distal Gap and Member Geometry

The spacing between the distal opening, flux lens, flux target, and guide-target member is not a cosmetic dimension. It is a magnetic design parameter. A small gap increases coupling to the high-permeability member but also increases the risk of shunting the field out of the chamber too early. A large gap preserves air-core behavior but reduces the target effect. The useful region is found by measuring distal B(t), radial divergence, and temperature while varying the gap.

In a compact foot applicator, a distal target gap of a few millimeters is a useful starting point. The lens can be close to the distal Coils because its function is perimeter shaping rather than bulk collection. The target can be placed slightly beyond the distal aperture so that the flux passes through the distal digital region before entering the target. The guide can begin at the target or near it, but the transition should avoid a narrow saturated neck.

The distal target area should generally be larger than the cross-section of a narrow guide strip. This is because the target first intercepts a spatially distributed field emerging from the treatment chamber. If all of that flux is forced immediately into a narrow strip, the strip can saturate. A wider target plate acts as a collector and distributor. It receives flux over a larger area and then feeds the return guide over a more manageable cross-section.

Corners and bends deserve special attention. In an L-shaped guide-target member, the transition between the upright plate and the base plate carries concentrated flux. A sharp bend can still work, but a radiused bend, a wider bend region, an increased local thickness, or a separate overlapping magnetic joint can reduce local saturation. For conductive materials, the bend region can also support eddy-current paths, so a slot pattern or laminated construction can be beneficial in high dB/dt embodiments.

The same tuning applies to axial guide placement. A guide should not be so close to the inner chamber that it steals treatment flux before that flux reaches tissue. It should not be so far away that it has little coupling to the return field. It should not form a closed conductive loop that creates unnecessary eddy-current heating. The correct guide geometry is therefore established by testing both magnetic field and temperature, not by visual symmetry alone.

Active guide tuning adds electrical variables to the mechanical gap variables. The guide-coil ampere-turns, lead time, overlap time, polarity, freewheel interval, high-impedance interval, and reset pulse can each change distal B(t). A useful guide-coil setting is therefore selected by measurement, not by assuming that maximum current gives maximum treatment flux. In some modes the best setting can be zero current with the guide coil open because the passive guide geometry already provides the desired return path.

### Mechanical Integration, Insulation, and User-Facing Surfaces

The flux-enhancement members are preferably integrated into the applicator in a way that does not compromise user safety or mechanical durability. The Coil conductors are ordinarily magnet wire with their own conductor insulation, and the flux guides, lenses, targets, guide-target members, and Flux-Guide Coils are ordinarily spatially separated from the Windings by molded plastic spacing, channels, standoffs, coatings, sleeves, bobbins, or comparable housing structure. The design point is reliable separation, retention, and positioning, not a special insulation scheme.

The user-facing treatment chamber should remain smooth and cleanable. The distal target is preferably outside the foot-contact region or separated from it by the chamber wall. If any high-permeability structure is close to a user-facing surface, the intervening wall should prevent sharp edges, corrosion products, heat concentration, or electrical exposure from reaching the user. The magnetic member should be captured so that repeated insertion and removal of a foot cannot dislodge it.

The presence of high-permeability members should also be considered in mechanical load paths. A user may rest weight on the applicator, especially in a foot device. The Coils should not be crushed or abraded by that load. The chamber support structure preferably transfers load around the Windings and into a housing, base, frame, or underlying surface. This allows the Windings to be selected for magnetic performance rather than mechanical load bearing.

Manufacturing repeatability matters because small changes in gap, alignment, or guide-coil position can affect the distal field. The housing can include molded stops that set the target gap, channels that position axial guides, ribs that hold the L-shaped member against a datum, bobbin features that position the Flux-Guide Coil, and features that prevent rotation or translation. These are not merely packaging details. They make the magnetic circuit and active guide coupling reproducible from unit to unit.

Serviceability is also improved by simple magnetic structures. A single L-shaped steel member can be inspected visually, placed in a housing, and retained by the same housing components that support the applicator core. It avoids complex moving parts, fluid cooling, user adjustments, and high-voltage applicator cables. The simplicity is part of the invention because it allows a meaningful distal flux enhancement to be used in a practical MPTD product.

### Support for Optional Combinations

The disclosed embodiments intentionally support several claimable combinations. A first combination uses a segmented solenoid and increased distal longitudinal turn density. A second combination adds overlapping Windings, commingled Windings, taper-controlled winding support, or segment-length control. A third combination adds a distal flux lens. A fourth combination adds a distal flux target. A fifth combination adds axial flux guides. A sixth combination adds a Flux-Guide Coil for active guide energization. A seventh combination integrates the target and guide into a single L-shaped or U-shaped member. An eighth combination adds temperature-responsive magnetic-flux optimization using temperature sensing, model data, thermal-budget determination, and adaptive pulse-control parameters.

The combinations are not arbitrary. Each solves a different part of the same distal-end problem. Distal turn density supplies more local magnetic drive. Overlapping or commingled Windings smooth or shape the axial ampere-turn distribution and can eliminate winding-free discontinuities that would otherwise produce leakage. The lens shapes the aperture. The target draws the field through the end. The guide returns the field. The Flux-Guide Coil actively controls when and how strongly a guide participates. The integrated member reduces part count and improves magnetic continuity. Temperature-responsive pulse control uses available thermal headroom to increase or optimize distal output, reduce delivery time, or improve total magnetic-flux dosage when the hardware can safely accept it. Together, these structures complement each other.

Because each feature has an independent function, the specification supports embodiments in which one feature is omitted. For example, a small applicator can use an L-shaped guide-target member without separate bar guides. A longer applicator can use solenoid elongation and a flux lens without a distal target if the distal field is already sufficient. A device optimized for maximum simplicity can use only a passive L-shaped member. A device optimized for active return-path control can add one or more Flux-Guide Coils to the L-shaped member, side guides, bottom guide, or other longitudinal ferromagnetic structures. A device optimized for maximum distal field can use elongation, LICD, overlapping Windings, lens, target, guides, active guide coils, tapered commingled winding construction, and temperature-responsive pulse control together.

The preferred commercial direction is the integrated guide-target member combined with distal turn-density shaping, tapered commingled winding construction, and temperature-responsive pulse control. In another preferred direction, the integrated guide-target member or selected side guides are further provided with Flux-Guide Coils so that the controller can actively energize, pre-bias, reset, or disable the external return path according to the selected treatment mode. These combinations provide a strong engineering balance: they address the field problem directly, use ordinary materials, occupy little additional space, and are easy to assemble. The broader disclosure is nevertheless important because the functional architecture is not limited to one exact piece of bent steel, one exact winding process, or one exact guide-coil timing sequence.

### Conclusion of the Detailed Description

The present invention should therefore be understood as a coordinated distal-end flux-management system for a solenoidal MPTD applicator used for pain treatment. The baseline segmented solenoid produces useful Core Flux in a hollow treatment chamber. The finite distal end of that solenoid naturally weakens and disorders the field near the distal digital region. The disclosed enhancements correct that weakness by moving the end effect, increasing distal excitation, smoothing or shaping axial ampere-turn distribution, reducing or eliminating winding-free discontinuities in tapered or commingled winding constructions, shaping the distal perimeter, providing a distal target, returning external flux through intentional high-permeability structures, optionally actively energizing selected external flux guides, and using temperature-responsive control to exploit available thermal headroom for improved flux delivery.

The specification gives the engineer the information needed to build and tune the system: the solenoid geometry, the distal problem, the magnetic-circuit principles, the guide, Flux-Guide Coil, lens, target, U-shaped and L-shaped embodiments, tapered and commingled winding construction, material choices, gap considerations, saturation concerns, eddy-current concerns, temperature-responsive pulse control, model-based thermal-budget control, guide-coil control, magnetic measurement method, and thermal validation method. These features define a set of distal-end enhancements that can be used individually or in selected combinations.

In one aspect, an applicator 100 for a magnetic pulse therapy device has distal ferromagnetic flux enhancement and includes a treatment chamber 225 configured to receive a foot 200 inserted distally into the treatment chamber 225, the insertion defining a distal insertion direction; a solenoid comprising a plurality of windings disposed around at least a portion of the treatment chamber 225, the solenoid having a distal-most winding extent defined by a distal-most longitudinal position occupied by any winding of the plurality of windings; and at least one ferromagnetic distal flux-enhancement structure selected from the group consisting of (a) a flux lens 610, (b) a flux target 1055, (c) an axial flux guide 605, and (d) a combination flux target and flux guide 1745. A distal reference position is the more proximal of (i) a distal-most longitudinal position of the treatment chamber 225 and (ii) the distal-most winding extent, and at least a portion of the ferromagnetic distal flux-enhancement structure is positioned distally of a reference plane perpendicular to the distal insertion direction and located 15 mm proximal of the distal reference position.

In another aspect, an applicator 100 for a magnetic pulse therapy device has distal solenoid elongation and includes a treatment chamber 225 configured to receive a foot 200 inserted distally into the treatment chamber 225, the insertion defining a distal insertion direction, and a solenoid comprising a plurality of windings disposed around at least a portion of the treatment chamber 225, the plurality of windings having a distal-most winding extent defined by a distal-most longitudinal position occupied by any winding of the plurality of windings. The distal-most winding extent is positioned at least 10 mm distal of a Reference Distal Point (RDP), as measured along the distal insertion direction. The RDP is the proximal-most of the following candidate points: (a) if the treatment chamber 225 includes a distal stop configured to limit distal movement of toes of the foot 200, a distal-most point of the distal stop; and (b) for each longitudinal position within a longitudinal extent of the treatment chamber 225, a point located distally of that longitudinal position by a distance D, in millimeters, where D=2.188H−37.1 and H is a chamber height, in millimeters, measured at a longitudinal centerline of the treatment chamber 225 at that longitudinal position.

In some embodiments, the candidate points further include additional candidate points, each additional candidate point being located 150 mm distal of a respective point of engagement at which the treatment chamber 225 is configured to limit insertion of the foot 200 by engaging a medial ankle region, a medial malleolar region, or a lower-leg region, as measured along the distal insertion direction.

In some embodiments, the applicator 100 further includes at least one ferromagnetic distal flux-enhancement structure selected from the group consisting of a flux lens 610, a flux target 1055, an axial flux guide 605, and a combination flux target and flux guide 1745, wherein at least a portion of the ferromagnetic distal flux-enhancement structure is positioned distally of a reference plane perpendicular to the distal insertion direction and located 15 mm proximal of a distal reference position, and wherein the distal reference position is the more proximal of (i) a distal-most longitudinal position of the treatment chamber 225 and (ii) the distal-most winding extent.

In some embodiments, the solenoid comprises a plurality of coils 105, and every turn of at least one coil 105 of the plurality of coils 105 is positioned distally of the RDP.

In another aspect, an applicator 100 having increased distal winding density for a magnetic pulse therapy device includes a treatment chamber 225 configured to receive a foot 200 inserted distally into the treatment chamber 225, the insertion defining a distal insertion direction, and a solenoid comprising one or more coils 105 collectively defining a plurality of turns disposed around at least a portion of the treatment chamber 225. The solenoid includes a distal winding-density region and a proximal reference region, the proximal reference region being proximal to and non-overlapping with the distal winding-density region. The distal winding-density region has a longitudinal length of at least 25 mm, measured along the distal insertion direction, and the proximal reference region has the same longitudinal length as the distal winding-density region. A longitudinal winding density of the distal winding-density region is at least 20% greater than a longitudinal winding density of the proximal reference region, and each longitudinal winding density is determined from turns or portions of turns of the one or more coils 105 that occupy the respective region, measured per unit longitudinal length.

In some embodiments, the applicator 100 further includes at least one ferromagnetic distal flux-enhancement structure selected from the group consisting of a flux lens 610, a flux target 1055, an axial flux guide 605, and a combination flux target and flux guide 1745, wherein the solenoid has a distal-most winding extent defined by a distal-most longitudinal position occupied by any turn of the plurality of turns, wherein a distal reference position is the more proximal of (i) a distal-most longitudinal position of the treatment chamber 225 and (ii) the distal-most winding extent, and wherein at least a portion of the ferromagnetic distal flux-enhancement structure is positioned distally of a reference plane perpendicular to the distal insertion direction and located 15 mm proximal of the distal reference position.

In another aspect, an applicator 100 having a distal ferromagnetic axial flux guide 605 for a magnetic pulse therapy device includes a treatment chamber 225 configured to receive a foot 200 inserted distally into the treatment chamber 225, the insertion defining a distal insertion direction; a solenoid comprising a plurality of windings disposed around at least a portion of the treatment chamber 225, the solenoid having a distal-most winding extent defined by a distal-most longitudinal position occupied by any winding of the plurality of windings; and at least one ferromagnetic axial flux guide 605 positioned outside the treatment chamber 225 and extending longitudinally along at least a portion of an exterior side region of the solenoid, the exterior side region being a lower, medial, lateral, or other exterior side region of the solenoid, the at least one ferromagnetic axial flux guide 605 being arranged to provide a lower-reluctance external return path for magnetic flux between a distal region and a proximal region of the solenoid. A distal reference position is the more proximal of (i) a distal-most longitudinal position of the treatment chamber 225 and (ii) the distal-most winding extent, and a distal portion of the at least one ferromagnetic axial flux guide 605 is positioned distally of a reference plane perpendicular to the distal insertion direction and located 15 mm proximal of the distal reference position.

In some embodiments, the at least one ferromagnetic axial flux guide 605 comprises at least one medial ferromagnetic axial flux guide 605 positioned on a medial exterior side of the solenoid relative to a foot 200 received in the treatment chamber 225 and at least one lateral ferromagnetic axial flux guide 605 positioned on a lateral exterior side of the solenoid relative to the foot 200 received in the treatment chamber 225.

In some embodiments, at least one guide selected from the at least one medial ferromagnetic axial flux guide 605 and the at least one lateral ferromagnetic axial flux guide 605 has a transverse width of 15 mm or less measured perpendicular to the distal insertion direction across the respective guide.

In some embodiments, the applicator 100 further includes a flux-guide coil 2255 comprising at least one winding disposed on, around, or electromagnetically coupled to the at least one ferromagnetic axial flux guide 605, and driver circuitry configured to energize the flux-guide coil 2255.

In some embodiments, the applicator 100 further includes a controller configured, during a treatment protocol, to operate the flux-guide coil 2255 in at least two operating modes selected from active energization before a magnetic pulse produced by the solenoid, active energization during at least part of a magnetic pulse produced by the solenoid, active energization in a first polarity, active energization in a second polarity opposite the first polarity, a de-energized or off state between magnetic pulses, a freewheeling state, a high-impedance state, an active clamping state, and an active discharge or reset state.

In another aspect, an applicator 100 having a distal ferromagnetic flux target 1055 for a magnetic pulse therapy device includes a treatment chamber 225 configured to receive a foot 200 inserted distally into the treatment chamber 225, the insertion defining a distal insertion direction; a solenoid comprising a plurality of windings disposed around at least a portion of the treatment chamber 225, the solenoid having a distal-most winding extent defined by a distal-most longitudinal position occupied by any winding of the plurality of windings; and a ferromagnetic flux target 1055 positioned outside the treatment chamber 225. A distal reference position is the more proximal of (i) a distal-most longitudinal position of the treatment chamber 225 and (ii) the distal-most winding extent, and at least a portion of the ferromagnetic flux target 1055 is positioned distally of a reference plane perpendicular to the distal insertion direction and located 15 mm proximal of the distal reference position.

In some embodiments, the applicator 100 further includes a ferromagnetic flux lens 610, and the ferromagnetic flux target 1055 is positioned distally of the ferromagnetic flux lens 610.

In some embodiments, the ferromagnetic flux target 1055 is part of a ferromagnetic combination flux target and flux guide 1745 comprising a base portion positioned along a lower exterior side region of the solenoid and extending longitudinally along the solenoid, and an upright distal portion forming the ferromagnetic flux target 1055.

In some embodiments, the ferromagnetic flux target 1055 is part of a ferromagnetic combination flux target and flux guide 1435 comprising a first longitudinal guide portion extending along a first exterior side region of the solenoid, a second longitudinal guide portion extending along a second exterior side region of the solenoid, and a distal flux-target portion extending around or across a distal region of the solenoid and magnetically coupling the first and second longitudinal guide portions.

In another aspect, a magnetic pulse therapy device having temperature-responsive magnetic-flux optimization in a foot-receiving solenoid applicator 100 includes a foot-receiving applicator 100 comprising a treatment chamber 225 configured to receive a foot 200 inserted distally into the treatment chamber 225, the insertion defining a distal insertion direction, and a solenoid comprising one or more windings disposed around at least a portion of the treatment chamber 225; at least one temperature sensor 2410 configured to sense a temperature of the foot-receiving applicator 100, the solenoid, or a component thermally coupled to the solenoid; and a controller configured to energize the solenoid during a treatment protocol to produce magnetic pulses in the treatment chamber 225, to receive temperature data from the at least one temperature sensor 2410, and to select a first setting of one or more magnetic-pulse-control parameters based at least in part on the temperature data, each magnetic-pulse-control parameter being selected from pulse timing, pulse duration, pulse repetition frequency, rest interval, selection of one or more windings, winding groups, taps, coils 105, or coil segments 205 to be energized, pulse waveform, forced-return enablement state at an end of a pulse, and, when the applicator 100 includes a flux-guide coil 2255, an operating state of the flux-guide coil 2255. The controller is configured, during the treatment protocol, to update the first setting to a second setting based at least in part on later temperature data received from the at least one temperature sensor 2410 so as to control magnetic flux delivered in the treatment chamber 225 while maintaining the sensed temperature at or below a temperature limit.

In some embodiments, the foot-receiving applicator 100 further comprises at least one ferromagnetic distal flux-enhancement structure selected from the group consisting of a flux lens 610, a flux target 1055, an axial flux guide 605, and a combination flux target and flux guide 1745, wherein the solenoid has a distal-most winding extent defined by a distal-most longitudinal position occupied by any winding of the one or more windings, wherein a distal reference position is the more proximal of (i) a distal-most longitudinal position of the treatment chamber 225 and (ii) the distal-most winding extent, and wherein at least a portion of the ferromagnetic distal flux-enhancement structure is positioned distally of a reference plane perpendicular to the distal insertion direction and located 15 mm proximal of the distal reference position.

In some embodiments, the controller is configured to store or access model data representing at least one relationship among one or more of the magnetic-pulse-control parameters, magnetic flux density delivered in the treatment chamber 225, and heat generation in the foot-receiving applicator 100, and the controller is configured to use the model data to select or update the one or more magnetic-pulse-control parameters to deliver a target magnetic-flux dosage during the treatment protocol while operating within a thermal budget determined at least in part from the temperature data.

In another aspect, an applicator 100 having solenoid coil segments 205 configured to permit winding commingling for a magnetic pulse therapy device includes a treatment chamber 225 configured to receive a foot 200 inserted distally into the treatment chamber 225, the insertion defining a distal insertion direction, and a solenoid comprising a plurality of separately energizable coil segments 205 disposed around at least a portion of the treatment chamber 225, the plurality of separately energizable coil segments 205 including a longitudinally adjacent pair of coil segments 205 arranged along a longitudinal direction of the solenoid, the longitudinally adjacent pair comprising a first coil segment 205 and a second coil segment 205, each of the first and second coil segments 205 comprising respective windings. The first and second coil segments 205 are wound on a shared winding-support structure and are arranged so that, at an interface region between the first and second coil segments 205, windings of the first and second coil segments 205 are permitted to nest, overlap, intermix, or otherwise commingle with one another.

In some embodiments, windings of the first and second coil segments 205 collectively form a winding mass along a superior side region of the treatment chamber 225, the superior side region being configured to face a dorsum of the foot 200 received in the treatment chamber 225, and, across the interface region, the winding mass is continuous along the superior side region without a winding-free discontinuity between adjacent portions of the winding mass, including without a winding-free discontinuity formed by a stair-step transition between axially adjacent winding stacks.

In some embodiments, the interface region includes an actual commingling zone in which windings of the first coil segment 205 and windings of the second coil segment 205 occupy overlapping longitudinal positions, the actual commingling zone has a longitudinal extent of at least 3 mm measured along the longitudinal direction of the solenoid, and, within the actual commingling zone, at least one winding portion of one of the first and second coil segments 205 is positioned at least 3 mm distal of at least one winding portion of the other of the first and second coil segments 205.

In some embodiments, the applicator 100 does not include a first bobbin carrying the first coil segment 205 and a separate second bobbin carrying the second coil segment 205.

In some embodiments, the plurality of separately energizable coil segments 205 comprises at least four coil segments 205 wound on the shared winding-support structure and arranged along the longitudinal direction of the solenoid.

In some embodiments, at least three longitudinally adjacent pairs among the at least four coil segments 205 each include a respective actual commingling zone in which windings of both coil segments 205 of the respective longitudinally adjacent pair occupy overlapping longitudinal positions, each respective actual commingling zone having a longitudinal extent of at least 3 mm measured along the longitudinal direction of the solenoid.

In some embodiments, within the actual commingling zone, windings of the first and second coil segments 205 are interleaved through a thickness of a winding mass formed by the windings of the first and second coil segments 205 and measured outward from the treatment chamber 225, such that a winding portion of one of the first and second coil segments 205 is positioned between winding portions of the other of the first and second coil segments 205 through the thickness of the winding mass.

In some embodiments, the shared winding-support structure includes a superior winding-support portion positioned between a superior boundary of the treatment chamber 225 and at least part of the solenoid, the superior winding-support portion carries windings of at least one of the plurality of separately energizable coil segments 205 and extends along a taper-measurement extent of at least 100 mm measured along the distal insertion direction, and, over the taper-measurement extent, a chamber height measured at a longitudinal centerline of the treatment chamber 225 between an inferior boundary of the treatment chamber 225 and the superior boundary of the treatment chamber 225 is at least 30 mm throughout the taper-measurement extent and increases by at least 40 mm from a distal end of the taper-measurement extent to a proximal end of the taper-measurement extent.

In some embodiments, in a longitudinal cross-section through the longitudinal centerline of the treatment chamber 225, the superior boundary of the treatment chamber 225 defines a curved profile over at least 50 mm of the taper-measurement extent, and a local rate of chamber-height increase along the curved profile does not decrease when moving proximally along the curved profile.

### PLANAR PLATE APPLICATOR EMBODIMENTS

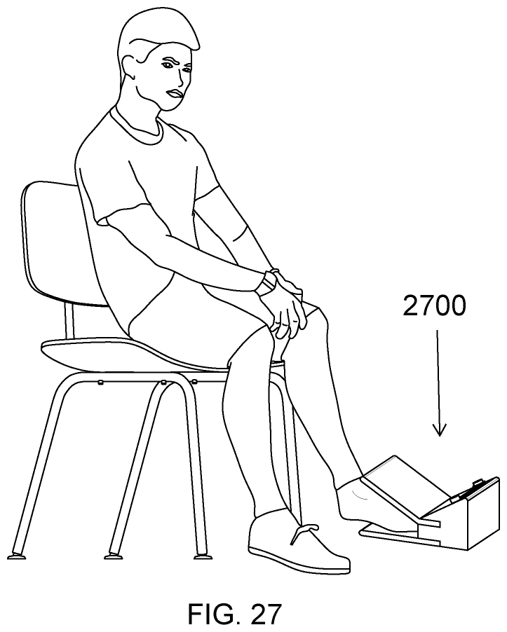

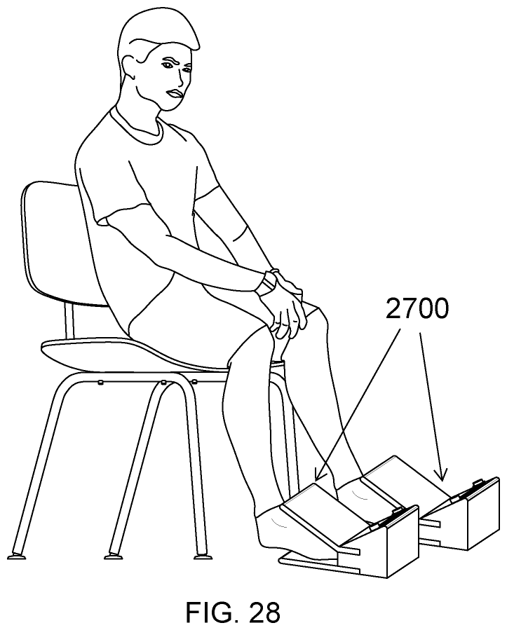

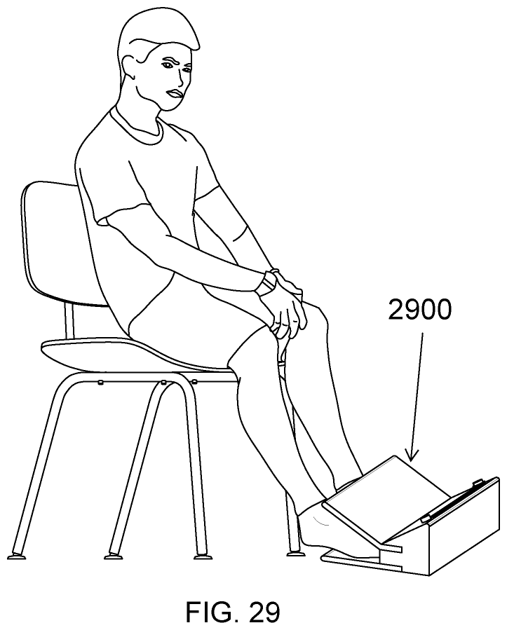

The following paragraphs describe planar plate applicator embodiments with reference to FIGS. 27-42. These embodiments may be practiced independently of, or in combination with, the solenoid applicator embodiments described above.

The present invention introduces a novel class of magnetic pulse therapy applicators termed a “planar plate” applicator. Unlike conventional coil/loop configurations or solenoid designs, a planar plate applicator introduces a new geometry wherein therapeutic magnetic flux is delivered between two flat, facing, magnetically permeable plates that serve as flux distribution surfaces. These two plates are magnetically coupled via a high-permeability core, forming an integrated flux circuit that is excited by one or more electromagnetic coils wound around the central core, analogous to a traditional electromagnet.

In operation, current through the excitation coil generates magnetic flux that enters the core and flows outward from the poles of the electromagnet into the respective plates. The high-permeability material of the plates facilitates broad lateral dispersion of the flux within each plate, allowing it to spread uniformly across the plate. The flux then exits each plate and traverses the air gap between them, entering the opposing plate in a substantially coherent and parallel path. The magnetic return path is completed through the core, resulting in a tightly coupled, closed-loop magnetic circuit that confines the majority of the flux within the active treatment volume between the plates.

This configuration yields a unique advantage: the magnetic field between the plates is highly uniform, coherent, and directionally aligned, similar to the flux observed along the core axis of a solenoid. However, in contrast to a solenoid, which typically treats cylindrical or tubular regions, the planar plate applicator defines a treatment zone with flat geometry—well suited for extremities such as feet or hands. Unlike loop-based applicators that emit multi-directional, disorganized flux with substantial off-target loss, the planar plate applicator ensures that nearly all generated flux passes through the intended treatment volume, minimizing inefficiencies and maximizing therapeutic flux density at target tissue depth.

Another key benefit of the planar plate design is the predictability of the field distribution. The geometry inherently avoids interference patterns, hotspots, or null regions that can arise from irregular field summation in loop applications. The uniform field enables uniform stimulation across the entire treated area, reducing the risk of overstimulation in localized zones and eliminating underexposed regions within the treatment volume. This makes it particularly suitable for protocols that require consistent dose delivery across a surface.

The planar plate applicator combines the core-directed efficiency of a solenoid with the broad-area coverage of loop systems, but without their respective limitations. Its flat form factor, strong internal coupling, and uniform flux distribution represent a significant advancement in magnetic pulse therapy, enabling highly efficient and spatially controlled treatment while maintaining design simplicity and manufacturability.

A further advantage of the planar plate applicator is its exceptional ease of use for patients in a home or clinical setting. While solenoid applicators are generally user-friendly—often requiring only that the user slide a limb, such as a foot, into the central bore of the coil—the planar plate design simplifies the process even further. The patient need only place the affected limb or body part onto a treatment surface, where it is positioned between two fixed, facing magnetic plates. This flat, open geometry eliminates the need for alignment within a confined coil and facilitates quick, accurate positioning without special training or adjustment. As a result, planar plate applicators are particularly well-suited for unsupervised or at-home therapeutic use, especially in populations with limited mobility.

A planar plate applicator 2700, 2900, 3600 consists fundamentally of two magnetically permeable plates 3020, 3030, 3810, 3820 positioned in opposition to one another, defining a treatment zone between them. These plates 3020, 3030, 3810, 3820 may be configured in a strictly parallel arrangement or oriented at a specific angle relative to each other, depending on the desired field-geometry or ergonomic considerations for patient placement. Each plate 3020, 3030, 3810, 3820 is magnetically coupled to one of the two poles of a common high-permeability core 3230, 3830 which serves as the magnetic return path and functional backbone of the system 2700, 2900, 3600.

The core 3230, 3830 itself forms the central element of a conventional electromagnet, around which one or more electromagnetic coils 3310, 4010 are wound. These coils typically consist of multiple turns of conductive wire, selected to achieve the desired inductance, current handling, and magnetic field strength. The winding configuration—such as single-layer, multi-layer, or segmented—may be optimized for thermal performance, space constraints, or driving waveform characteristics. When energized, the coils generate magnetic flux that flows into the core 3230, 3830 then into the attached plates 3020, 3030, 3810, 3820, enabling controlled delivery of pulsed electromagnetic fields across the volume between the plates.

The specific configuration of the electromagnetic coils used in the planar plate applicator 2700, 2900, 3600 is not unique to this invention and may vary according to standard engineering practices. Coil design—including parameters such as number of turns, wire gauge, core material, and winding topology—can be selected based on the intended electrical characteristics such as voltage, current, inductance, and desired pulse profile. These parameters may be adapted to accommodate a range of commercially available or custom-designed pulse generators. For example, a single high-voltage, high-amperage coil may be employed for use with traditional capacitor-discharge pulse systems, while in other implementations, the coil may take the form of a segmented solenoid as disclosed in U.S. Pat. No. '891, enabling operation at lower voltages with reduced electromagnetic interference and improved safety.

It is important to note that the present invention is not directed to the design of the coil 3310, 4010 itself, but rather to the novel method of magnetic flux distribution achieved through the use of the planar plate geometry. The innovation lies in how the magnetic field, once generated by the excitation coil and directed through the core 3230, 3830 is dispersed laterally and uniformly into the plates 3020, 3030, 3810, 3820 and across the air gap between them. This flux geometry results in a highly efficient, uniform, and coherent treatment field, distinguishing the invention from loop and solenoid applicators regardless of the electrical or structural details of the driving coil.

One of the critical aspects of the planar plate applicator 2700, 2900, 3600 is the magnetic coupling between the electromagnet core and the two opposing plates. For the applicator to function effectively, this magnetic connection must exhibit high permeability and minimal magnetic reluctance to ensure efficient flux transfer. Even a very small air gap—less than 1 mm, or in many cases less than 0.1 mm—can introduce significant magnetic reluctance, substantially impeding the ability of flux to pass from the core into the plates. This resistance to magnetic flow reduces the total field strength delivered into the treatment region and degrades overall applicator performance.

Therefore, the physical and magnetic connection between the core and each plate must be designed to minimize or eliminate any discontinuity in the magnetic path. While it is possible to mechanically attach the plates to the core via fillet welding, adhesives, or mechanical fasteners, such approaches typically result in imperfect interfaces with high reluctance, especially when non-magnetic bonding materials or thermal stress effects disrupt continuity. Fillet welds or mechanical press fits, although structurally robust, often fail to provide a magnetically optimal interface.

The second key factor in the design of a successful planar plate applicator 2700, 2900, 3600 is the management of magnetic saturation. During operation, magnetic pulse therapy devices typically generate very high-intensity pulses, often reaching magnetic flux densities of B=2 tesla or greater within the core. This intense flux must be transferred into the planar plates and then across the treatment zone without reaching saturation levels in any part of the magnetic circuit. If saturation occurs—either in the core or in the plates—the material's permeability collapses, causing flux to divert unpredictably, decreasing field strength in the treatment zone and compromising the uniformity and efficiency of the applicator.

Preventing saturation requires careful material selection and appropriate dimensional design. The first approach is to utilize high-permeability materials throughout the magnetic path. Pure iron, for example, exhibits high magnetic permeability and a relatively high saturation point, making it ideal for both the core and plates. High-permeability materials ensure that magnetic flux is channeled efficiently away from the coil and into the plates with minimal energy loss or field distortion.

The second approach is to ensure that both the core and plates have sufficient cross-sectional area to carry the total flux without exceeding their saturation limits. Even when lower-permeability materials such as mild steel (e.g., AISI 1018) are used due to cost, availability, or fabrication considerations, saturation can be avoided by increasing the material thickness to distribute the flux over a larger volume. This compensates for the reduced permeability and prevents localized saturation. In all cases, magnetic modeling and empirical verification are necessary to ensure that the chosen geometry and material combination maintains linear magnetic behavior over the full range of operational pulse intensities.

Another important design consideration for the planar plate applicator 2700, 2900, 3600 is the shape and size of the plates themselves. Once magnetic flux enters each plate from the core, it disperses laterally across the plate's surface area. The extent of this dispersion is governed by the plate's geometry and the material's magnetic permeability. As the flux spreads, the local magnetic flux density (B) decreases proportionally with increasing surface area. Thus, the larger the plate, the more the flux is diluted, reducing the effective field strength delivered across the treatment zone.

To maintain therapeutically effective flux densities—particularly in systems operating near saturation thresholds—it is critical to size the plates appropriately. The ideal plate size is one that closely matches the cross-sectional profile of the intended treatment target. For example, if the applicator 2700, 2900, 3600 is intended for use on a human foot, the plates should be shaped and dimensioned to conform to the side or bottom projection of a foot, possibly with a margin of extension to accommodate positional variation. Oversized plates, while increasing coverage, may lower field strength below effective therapeutic thresholds unless driven by significantly more powerful coils, which in turn increases cost, power requirements, and heat generation. Therefore, optimal performance is achieved by carefully matching plate dimensions to the anatomical target, balancing coverage area with flux density to preserve field uniformity and efficacy.

The spacing between the planar plates is a critical design parameter that directly impacts both magnetic efficiency and field uniformity. As the distance between the two plates increases, the magnetic circuit must span a larger air gap, which introduces substantial magnetic reluctance. Air, having extremely low magnetic permeability relative to ferromagnetic materials, acts as a bottleneck in the magnetic path. This increased reluctance causes a sharp reduction in magnetic flux density in the region between the plates. In many practical implementations, this reduction follows a nonlinear relationship—often approximating an inverse square or inverse cubic dependency on distance—meaning that even small increases in plate separation can drastically reduce effective B-field levels in the treatment zone.

In addition to reducing field strength, excessive spacing between the plates also compromises flux confinement. As the air gap grows, a greater portion of the magnetic flux begins to diverge from the intended path and instead loops through surrounding air, forming field lines that resemble those of a conventional axial electromagnet. This shift in field geometry results in a less coherent, less efficient, and more diffuse treatment field. The uniform, parallel flux lines characteristic of a well-designed planar plate applicator 2700, 2900, 3600 are replaced by arcing, radial field lines that not only reduce efficiency but may also produce uneven or poorly directed stimulation. To preserve the benefits of coherent, directed flux and minimize flux leakage or distortion, the spacing between plates should be minimized and optimized specifically for the anatomical thickness of the target body part, such as a foot, hand, or limb segment, while allowing adequate clearance for comfort and positioning.

At shorter magnetic pulse durations—typically in the range of tens to hundreds of microseconds—the ability of magnetic flux to distribute uniformly across the entire volume of each planar plate becomes limited due to eddy current effects and magnetic diffusion lag. In this regime, flux does not have sufficient time to fully permeate the lateral extent of the plate material, resulting in non-uniform flux distribution and reduced field effectiveness in portions of the treatment zone. This effect is especially pronounced in thinner plates, where the limited cross-sectional area constrains the magnetic path and exacerbates localized saturation or flux crowding near the core interface.

A practical and effective method for mitigating this limitation is to increase the thickness of the planar plates. Thicker plates provide a larger magnetic conduction path and a greater internal volume through which flux can redistribute more evenly, even under high dB/dt conditions associated with short-duration pulses. By increasing thickness, the plates behave more like magnetic reservoirs, buffering and diffusing incoming flux to ensure a more consistent field across the active treatment surface. This approach preserves the spatial uniformity and therapeutic integrity of the flux between the plates, allowing the applicator 2700, 2900, 3600 to maintain its advantages in coherence and efficiency, even when driven by high-speed pulse generators.

An important and non-obvious design feature of planar plate applicators is the ability to intentionally orient the plates at non-parallel angles relative to one another for advantage. This geometrical modification can be leveraged to create controlled gradients in magnetic flux density across the treatment zone. Because the magnetic flux between the plates is concentrated across the shortest distance, areas where the plates are closer together will naturally exhibit higher magnetic flux density, while areas with a wider gap will experience proportionally lower flux density due to increased magnetic reluctance and field dispersion.

This principle is particularly advantageous in anatomical applications where the treatment target has a non-uniform shape, such as the human foot. When treating the foot with one plate placed below the plantar surface and another above the dorsum, angling the top plate to match the natural slope of the dorsum—typically around 25 degrees—enables a more tailored and therapeutically effective flux distribution. In this configuration, the plate separation is smallest near the distal end (toes), resulting in higher localized flux density, and gradually increases toward the proximal end (inner-ankle), where lower flux density is appropriate. This graduated field intensity can enhance therapeutic targeting by delivering greater stimulation to nerve-rich distal regions while avoiding overstimulation of the more proximal structures. Thus, intentional plate angling offers a means of spatial flux modulation without increasing circuit complexity or power demands.

### EXEMPLARY EMBODIMENT

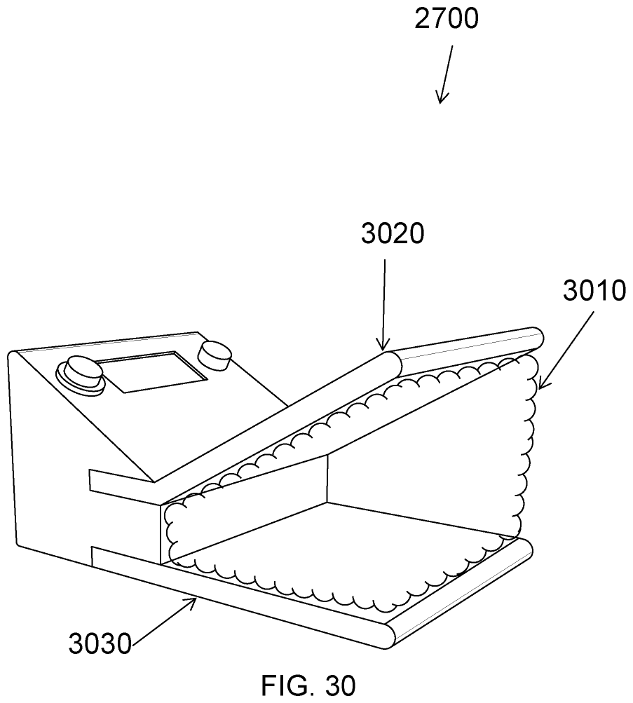

FIG. 30 illustrates one exemplary embodiment of the planar plate applicator 2700 specifically configured for the treatment of peripheral neuropathy in the foot. In this embodiment, a patient inserts the foot into the treatment zone 3010 such that one planar plate 3020 is positioned above the dorsum (top) of the foot and a second planar plate 3030 is positioned below the plantar (bottom) surface. This orientation ensures that the magnetic flux travels coherently and uniformly from one plate to the other, passing directly through the foot tissues in the vertical direction. The spatial configuration and gap 3010 are optimized to closely conform to the anatomical shape of a human foot, thereby maximizing therapeutic field coverage and minimizing flux leakage. The cloud callout 3010 is an approximation of the treatment zone for illustrative purposes and does not represent the limitations of the magnetic flux coverage of an actual device.

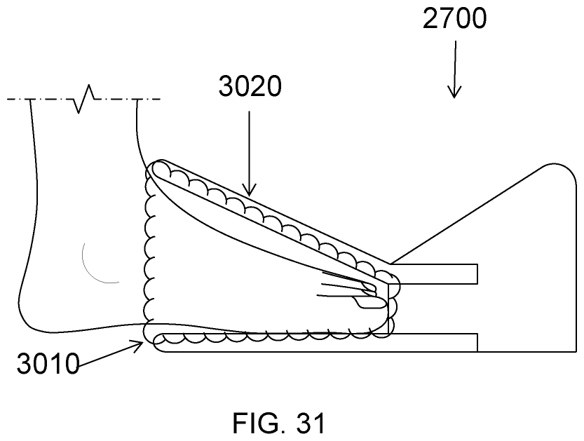

FIG. 31 provides a sectional view of the same applicator 2700 configuration with the foot in place, clearly showing the alignment of the foot within the active treatment volume 3010. This view demonstrates how the angled upper 3020 plate follows the natural slope of the foot dorsum to produce a flux density gradient favoring the distal regions, which are often most affected by neuropathy.

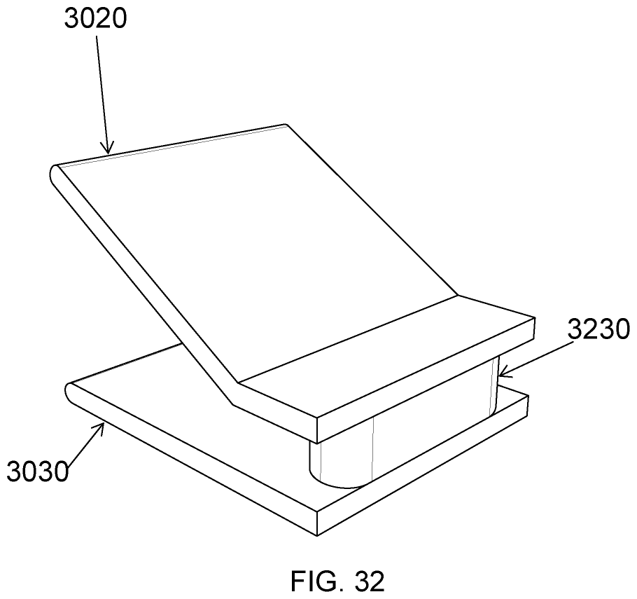

FIG. 32 depicts the structural magnetic assembly in isolation, with the housing, electronics, and coils removed for clarity. This figure shows the mechanical and magnetic architecture of the two plates 3020, 3030 and their connection to the central core 3230, forming a continuous low-reluctance magnetic circuit.

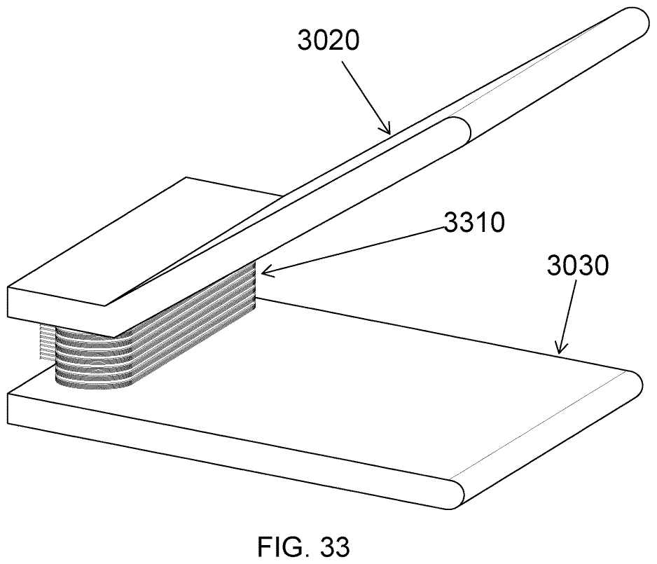

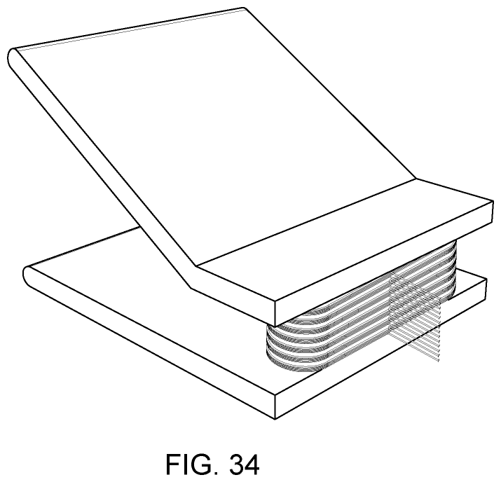

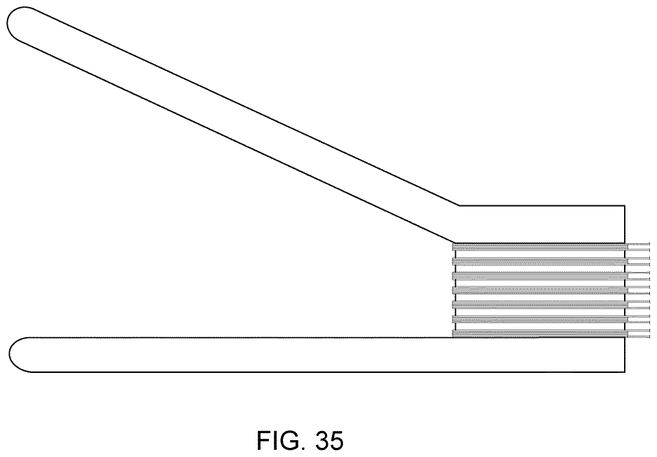

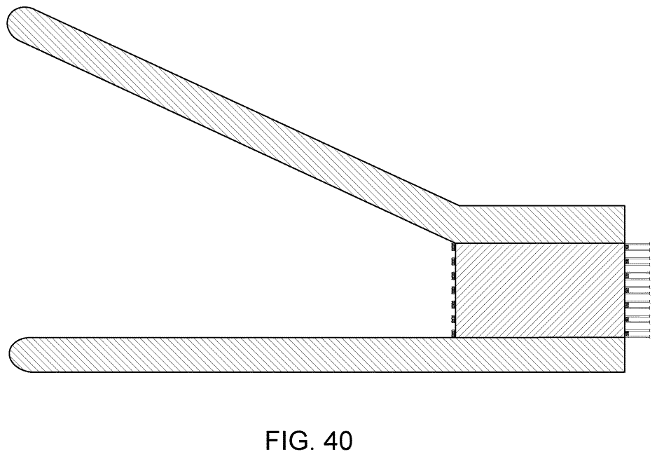

FIG. 33, FIG. 34, FIG. 35 and FIG. 40 build upon FIG. 32 by illustrating the full magnetic assembly with the electromagnetic coils 3310 wound around the core 3230. The coils are arranged concentrically and are electrically connected to an external pulse generator. When energized, the coils induce magnetic flux in the core 3230, which is then distributed into the plates 3020, 3030 and across the foot. This configuration ensures efficient flux transfer, minimal saturation, and uniform field delivery for therapeutic efficacy.

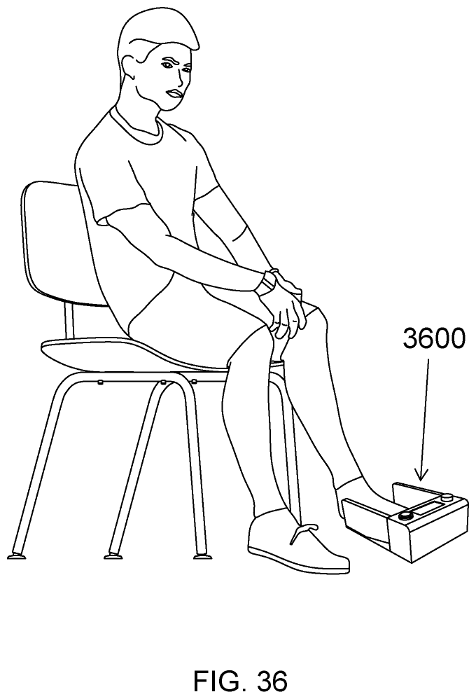

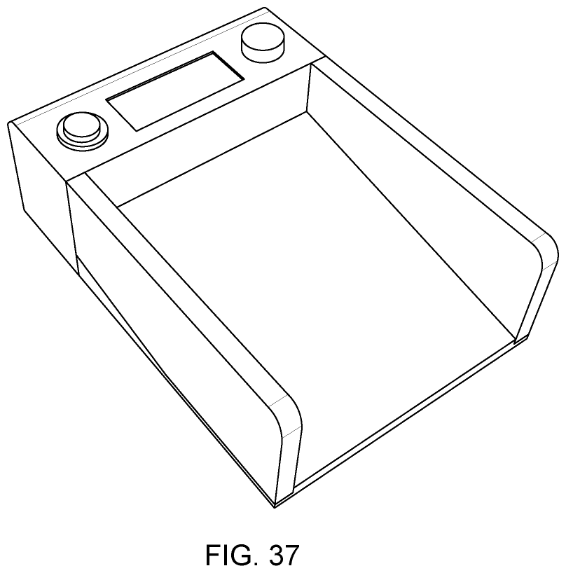

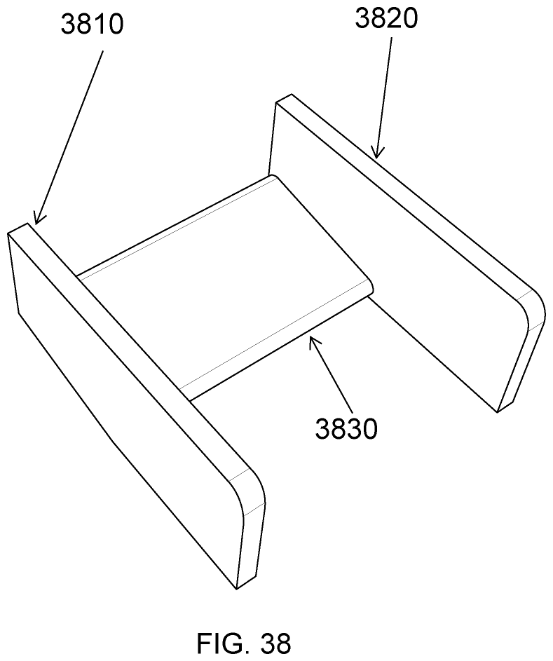

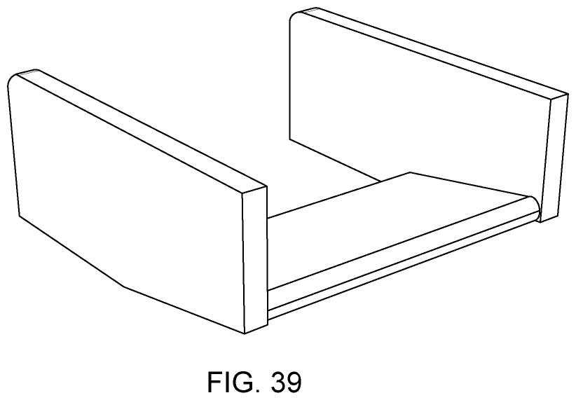

FIG. 36, FIG. 37, FIG. 38, and FIG. 39 illustrate another exemplary embodiment of a planar plate applicator 3600 wherein the upper and lower planar plates 3020, 3030 from earlier embodiments have been replaced by side plates 3810, 3830.

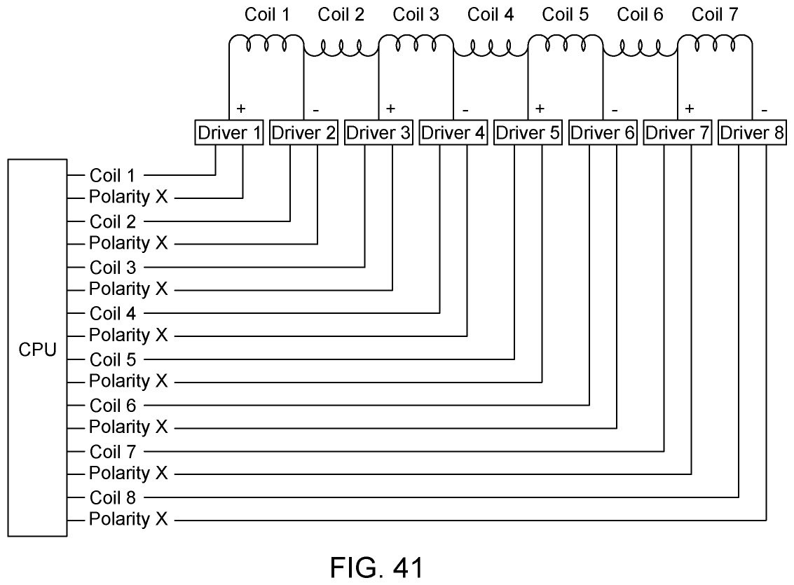

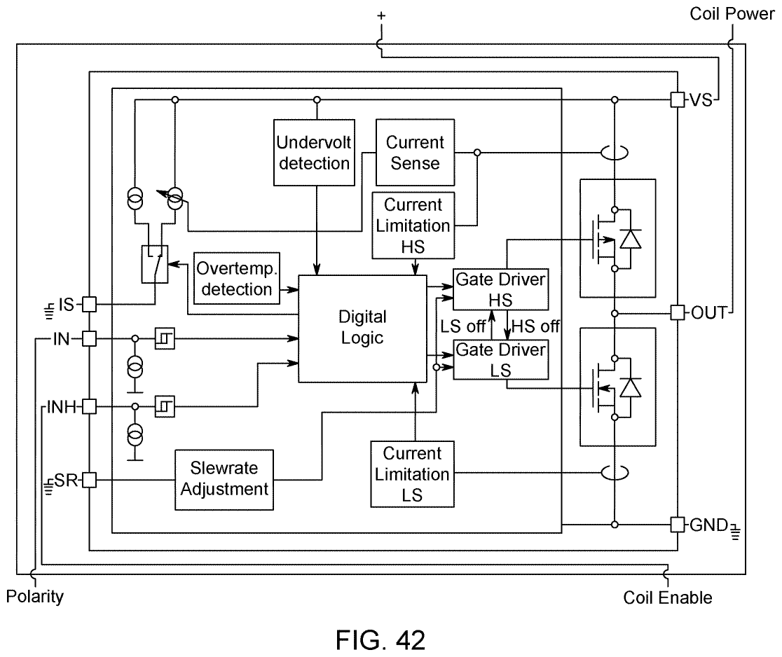

FIG. 41 and FIG. 42 are exemplary schematics for controlling a planar plate applicator 2700, 2900, 3600.

Successful prototype models of the planar plate applicator have been constructed using AISI 1018 low-carbon steel, a readily available and machinable ferromagnetic material with moderate magnetic permeability. In these models, both the upper and lower planar plates were fabricated from ½-inch thick AISI 1018 steel, while the central electromagnet core 3230 was constructed from 1-inch thick AISI 1018 steel to provide greater magnetic cross-sectional area and reduce the likelihood of core saturation.

To achieve magnetic continuity between the core 3230 and each plate 3020, 3030, full penetration welds were employed at the junctions. While this welding method does not provide the same magnetic performance as precision-machined, high-permeability monolithic connections, it proved sufficient for functional operation. The full penetration welds ensured mechanical integrity and created a continuous metallic path for flux transfer, enabling successful delivery of coherent magnetic fields between the two plates. Despite the moderate permeability of AISI 1018, the use of increased thickness in both the core 3230 and the plates effectively compensated for material limitations, avoiding saturation and allowing therapeutic flux densities to be achieved across the treatment zone.

This configuration was evaluated in a controlled laboratory setting and demonstrated the ability to generate therapeutically effective magnetic flux densities across the targeted treatment region. Measurements taken within the treatment zone showed that the flux density at the distal end of the applicator—corresponding to the region of the toes—ranged from approximately 35 to 50 millitesla (mT), depending on the coil geometry and power settings used; both higher and lower flux densities are achievable with appropriate adjustments. At the proximal end, near the ankle, flux density correspondingly measured between 10 to 15 mT. This graded field strength is particularly advantageous for the treatment of peripheral neuropathy, as the distal regions of the foot—such as the toes and forefoot—are typically the most affected by nerve damage and benefit most from higher-intensity stimulation. The gradual decrease in field intensity toward the proximal end helps avoid overstimulation of less-affected areas while maintaining a coherent and efficient field profile across the treatment volume. This spatial flux distribution aligns well with therapeutic requirements and reinforces the clinical utility of the planar plate applicator design.

While the previously described welded AISI 1018 steel configuration is suitable for rapid prototyping and functional validation, it is labor-intensive to fabricate and relies on a material that is magnetically sub-optimal. For production-scale manufacturing, a different approach used may be sintering. Sintering is an established metallurgical process ideally suited for creating high-precision, magnetically efficient components in large volumes with consistent quality.

In the sintering process, finely powdered metal—typically a high-permeability alloy or pure iron—is compacted into a precisely shaped mold under extremely high pressure. This pressure causes the particles to mechanically bond into a rigid, pre-formed shape known as a “green part.” The green part is then subjected to a high-temperature furnace cycle, typically in a controlled atmosphere. During this phase, the metal particles undergo solid-state diffusion, which fuses the particles at their boundaries without melting the entire structure. The result is a dense, solid metal component with excellent mechanical integrity and tailored magnetic properties.

Sintering offers several key advantages for this application: (1) it enables production of complex geometries with tight dimensional tolerances; (2) it eliminates the need for high-reluctance welded joints; and (3) it allows for the use of optimized materials with very high magnetic permeability and saturation thresholds. These factors collectively improve the magnetic efficiency, reduce material losses, and enhance the manufacturability and consistency of the planar plate applicator. As such, sintering is one method for fabricating the plates and core elements in a production environment.

This invention discloses a novel type of magnetic pulse therapy applicator, termed a planar plate applicator, which introduces a new category of field delivery distinct from traditional loop coils and solenoids. The core structural element comprises two ferromagnetic plates positioned to face each other, either in a parallel or angular configuration. These plates are magnetically coupled to the two poles of an electromagnetic core. The core itself is a solid high-permeability structure, around which one or more electrical coils are wound in accordance with conventional electromagnetic design practices. When energized, the coil structure generates a magnetic field that exits one pole of the electromagnet, enters one plate, and travels across the inter-plate gap to the opposite plate, completing the magnetic circuit through the other pole of the core.

The effectiveness of this architecture depends critically on low-reluctance magnetic coupling between the core and the planar plates. This is achieved by selecting materials with high magnetic permeability and avoiding interfaces that introduce excessive reluctance, such as poor welds or loose mechanical bonds. For optimal performance, the connections between the core and the plates must be continuous and magnetically conductive. The plates themselves must be of sufficient thickness to accommodate high magnetic pulse intensities without saturation and to allow uniform flux distribution across their surfaces. Materials such as pure iron or specialized soft magnetic alloys are used for their high saturation thresholds and low coercivity.

Unlike typical bipolar or axial electromagnets, where magnetic flux tends to disperse unpredictably into surrounding air or materials, the planar plate applicator forms a controlled and efficient magnetic circuit. Because the plates are positioned closer to each other than to any alternative return paths, magnetic flux is channeled predominantly and predictably between the two plates. This results in a treatment field that is coherent, aligned, and spatially uniform across the entire area between the plates. The design effectively minimizes stray flux and maximizes field utilization, making it especially suitable for medical and therapeutic applications where consistency and precision of flux exposure are critical.

Flux density between the plates can be precisely modulated by adjusting the inter-plate spacing. A narrower gap produces higher flux density, while a wider gap results in reduced intensity. This principle is applied advantageously in various disclosed embodiments for treating peripheral neuropathy of the foot. In some embodiments, the plates are arranged in a V-shaped configuration, with the distal end (towards the toes) having a smaller gap than the proximal end (near the ankle). This results in a gradient magnetic field where the highest flux densities occur in the forefoot, which is typically the most affected area in peripheral neuropathy patients, while lower intensities are delivered to less-affected proximal regions.

This architecture offers the advantages of solenoidal flux coherence without requiring the enclosing geometry of a traditional solenoid. At the same time, it avoids the field inefficiencies and hot spots commonly associated with loop-style applicators. By concentrating flux between two plates and tailoring field strength through geometry, the planar plate applicator delivers a high level of therapeutic precision, manufacturability, and user convenience—representing a significant advancement in the field of magnetic pulse therapy.

1. An applicator for a magnetic pulse therapy device, the applicator having distal ferromagnetic flux enhancement and comprising:
a treatment chamber configured to receive a foot inserted distally into the treatment chamber, the insertion defining a distal insertion direction;
a solenoid comprising a plurality of windings disposed around at least a portion of the treatment chamber, the solenoid having a distal-most winding extent defined by a distal-most longitudinal position occupied by any winding of the plurality of windings; and
at least one ferromagnetic distal flux-enhancement structure selected from:
(a) a flux lens;
(b) a flux target;
(c) an axial flux guide; and
(d) a combination flux target and flux guide;
wherein a distal reference position is the more proximal of (i) a distal-most longitudinal position of the treatment chamber and (ii) the distal-most winding extent; and
wherein at least a portion of the ferromagnetic distal flux-enhancement structure is positioned distally of a reference plane perpendicular to the distal insertion direction and located 15 mm proximal of the distal reference position.

2. An applicator for a magnetic pulse therapy device, the applicator having distal solenoid elongation and comprising:
a treatment chamber configured to receive a foot inserted distally into the treatment chamber, the insertion defining a distal insertion direction; and
a solenoid comprising a plurality of windings disposed around at least a portion of the treatment chamber, the plurality of windings having a distal-most winding extent defined by a distal-most longitudinal position occupied by any winding of the plurality of windings;
wherein the distal-most winding extent is positioned at least 10 mm distal of a Reference Distal Point (RDP), as measured along the distal insertion direction;
wherein the RDP is the proximal-most of the following candidate points:
(a) if the treatment chamber includes a distal stop configured to limit distal movement of toes of the foot, a distal-most point of the distal stop; and
(b) for each longitudinal position within a longitudinal extent of the treatment chamber, a point located distally of that longitudinal position by a distance D, in millimeters, where D=2.188H−37.1 and H is a chamber height, in millimeters, measured at a longitudinal centerline of the treatment chamber at that longitudinal position.

3. The applicator of claim 2, wherein the RDP further includes additional candidate points, each additional candidate point being located 150 mm distal of a respective point of engagement at which the treatment chamber is configured to limit insertion of the foot by engaging a medial ankle region, a medial malleolar region, or a lower-leg region, as measured along the distal insertion direction.

4. The applicator of claim 2, further comprising at least one ferromagnetic distal flux-enhancement structure selected from a flux lens, a flux target, an axial flux guide, and a combination flux target and flux guide, wherein at least a portion of the ferromagnetic distal flux-enhancement structure is positioned distally of a reference plane perpendicular to the distal insertion direction and located 15 mm proximal of a distal reference position, and wherein the distal reference position is the more proximal of (i) a distal-most longitudinal position of the treatment chamber and (ii) the distal-most winding extent.

5. The applicator of claim 2, wherein the solenoid comprises a plurality of coils, and wherein every turn of at least one coil of the plurality of coils is positioned distally of the RDP.

6. An applicator having increased distal winding density for a magnetic pulse therapy device, the applicator comprising:
a treatment chamber configured to receive a foot inserted distally into the treatment chamber, the insertion defining a distal insertion direction; and
a solenoid comprising one or more coils collectively defining a plurality of turns disposed around at least a portion of the treatment chamber;
wherein the solenoid includes a distal winding-density region and a proximal reference region, the proximal reference region being proximal to and non-overlapping with the distal winding-density region;
wherein the distal winding-density region has a longitudinal length of at least 25 mm, measured along the distal insertion direction, and the proximal reference region has the same longitudinal length as the distal winding-density region;
wherein a longitudinal winding density of the distal winding-density region is at least 20% greater than a longitudinal winding density of the proximal reference region; and
wherein each longitudinal winding density is determined from turns or portions of turns of the one or more coils that occupy the respective region, measured per unit longitudinal length.

7. The applicator of claim 6, further comprising at least one ferromagnetic distal flux-enhancement structure selected from a flux lens, a flux target, an axial flux guide, and a combination flux target and flux guide, wherein the solenoid has a distal-most winding extent defined by a distal-most longitudinal position occupied by any turn of the plurality of turns, wherein a distal reference position is the more proximal of (i) a distal-most longitudinal position of the treatment chamber and (ii) the distal-most winding extent, and wherein at least a portion of the ferromagnetic distal flux-enhancement structure is positioned distally of a reference plane perpendicular to the distal insertion direction and located 15 mm proximal of the distal reference position.

8. An applicator having a distal ferromagnetic axial flux guide for a magnetic pulse therapy device, the applicator comprising:
a treatment chamber configured to receive a foot inserted distally into the treatment chamber, the insertion defining a distal insertion direction;
a solenoid comprising a plurality of windings disposed around at least a portion of the treatment chamber, the solenoid having a distal-most winding extent defined by a distal-most longitudinal position occupied by any winding of the plurality of windings; and
at least one ferromagnetic axial flux guide positioned outside the treatment chamber and extending longitudinally along at least a portion of an exterior side region of the solenoid, the exterior side region being a lower, medial, lateral, or other exterior side region of the solenoid, the at least one ferromagnetic axial flux guide being arranged to provide a lower-reluctance external return path for magnetic flux between a distal region and a proximal region of the solenoid;
wherein a distal reference position is the more proximal of:
(i) a distal-most longitudinal position of the treatment chamber and
(ii) the distal-most winding extent; and
wherein a distal portion of the at least one ferromagnetic axial flux guide is positioned distally of a reference plane perpendicular to the distal insertion direction and located 15 mm proximal of the distal reference position.

9. The applicator of claim 8, wherein the at least one ferromagnetic axial flux guide comprises at least one medial ferromagnetic axial flux guide positioned on a medial exterior side of the solenoid relative to a foot received in the treatment chamber and at least one lateral ferromagnetic axial flux guide positioned on a lateral exterior side of the solenoid relative to the foot received in the treatment chamber.

10. The applicator of claim 9, wherein at least one guide selected from the at least one medial ferromagnetic axial flux guide and the at least one lateral ferromagnetic axial flux guide has a transverse width of 15 mm or less measured perpendicular to the distal insertion direction across the respective guide.

11. The applicator of claim 8, further comprising a flux-guide coil comprising at least one winding disposed on, around, or electromagnetically coupled to the at least one ferromagnetic axial flux guide, and driver circuitry configured to energize the flux-guide coil.

12. The applicator of claim 11, further comprising a controller configured, during a treatment protocol, to operate the flux-guide coil in at least two operating modes selected from active energization before a magnetic pulse produced by the solenoid, active energization during at least part of a magnetic pulse produced by the solenoid, active energization in a first polarity, active energization in a second polarity opposite the first polarity, a de-energized or off state between magnetic pulses, a freewheeling state, a high-impedance state, an active clamping state, and an active discharge or reset state.

13. An applicator having a distal ferromagnetic flux target for a magnetic pulse therapy device, the applicator comprising:
a treatment chamber configured to receive a foot inserted distally into the treatment chamber, the insertion defining a distal insertion direction;
a solenoid comprising a plurality of windings disposed around at least a portion of the treatment chamber, the solenoid having a distal-most winding extent defined by a distal-most longitudinal position occupied by any winding of the plurality of windings; and
a ferromagnetic flux target positioned outside the treatment chamber;
wherein a distal reference position is the more proximal of:
(i) a distal-most longitudinal position of the treatment chamber and
(ii) (ii) the distal-most winding extent; and
wherein at least a portion of the ferromagnetic flux target is positioned distally of a
reference plane perpendicular to the distal insertion direction and located 15 mm proximal of the distal reference position.

14. The applicator of claim 13, further comprising a ferromagnetic flux lens, wherein the ferromagnetic flux target is positioned distally of the ferromagnetic flux lens.

15. The applicator of claim 13, wherein the ferromagnetic flux target is part of a ferromagnetic combination flux target and flux guide, the ferromagnetic combination flux target and flux guide comprising a base portion positioned along a lower exterior side region of the solenoid and extending longitudinally along the solenoid, and an upright distal portion forming the ferromagnetic flux target.

16. The applicator of claim 13, wherein the ferromagnetic flux target is part of a ferromagnetic combination flux target and flux guide, the ferromagnetic combination flux target and flux guide comprising a first longitudinal guide portion extending along a first exterior side region of the solenoid, a second longitudinal guide portion extending along a second exterior side region of the solenoid, and a distal flux-target portion extending around or across a distal region of the solenoid and magnetically coupling the first and second longitudinal guide portions.

17. A magnetic pulse therapy device having temperature-responsive magnetic-flux optimization in a foot-receiving solenoid applicator, comprising:
a foot-receiving applicator comprising a treatment chamber configured to receive a foot inserted distally into the treatment chamber, the insertion defining a distal insertion direction, and a solenoid comprising one or more windings disposed around at least a portion of the treatment chamber;
at least one temperature sensor configured to sense a temperature of the foot-receiving applicator, the solenoid, or a component thermally coupled to the solenoid; and
a controller configured to energize the solenoid during a treatment protocol to produce magnetic pulses in the treatment chamber, to receive temperature data from the at least one temperature sensor, and to select a first setting of one or more magnetic-pulse-control parameters based at least in part on the temperature data, each magnetic-pulse-control parameter being selected from pulse timing, pulse duration, pulse repetition frequency, rest interval, selection of one or more windings, winding groups, taps, coils, or coil segments to be energized, pulse waveform, forced-return enablement state at an end of a pulse, and, when the applicator includes a flux-guide coil, an operating state of the flux-guide coil;
wherein the controller is configured, during the treatment protocol, to update the first setting to a second setting based at least in part on later temperature data received from the at least one temperature sensor so as to control magnetic flux delivered in the treatment chamber while maintaining the sensed temperature at or below a temperature limit.

18. The magnetic pulse therapy device of claim 17, wherein the foot-receiving applicator further comprises at least one ferromagnetic distal flux-enhancement structure selected from a flux lens, a flux target, an axial flux guide, and a combination flux target and flux guide, wherein the solenoid has a distal-most winding extent defined by a distal-most longitudinal position occupied by any winding of the one or more windings, wherein a distal reference position is the more proximal of (i) a distal-most longitudinal position of the treatment chamber and (ii) the distal-most winding extent, and wherein at least a portion of the ferromagnetic distal flux-enhancement structure is positioned distally of a reference plane perpendicular to the distal insertion direction and located 15 mm proximal of the distal reference position.

19. The magnetic pulse therapy device of claim 17, wherein the controller is configured to store or access model data representing at least one relationship among one or more of the magnetic-pulse-control parameters, magnetic flux density delivered in the treatment chamber, and heat generation in the foot-receiving applicator, and wherein the controller is configured to use the model data to select or update the one or more magnetic-pulse-control parameters to deliver a target magnetic-flux dosage during the treatment protocol while operating within a thermal budget determined at least in part from the temperature data.

20. An applicator having solenoid coil segments configured to permit winding commingling for a magnetic pulse therapy device, the applicator comprising:
a treatment chamber configured to receive a foot inserted distally into the treatment chamber, the insertion defining a distal insertion direction; and
a solenoid comprising a plurality of separately energizable coil segments disposed around at least a portion of the treatment chamber, the plurality of separately energizable coil segments including a longitudinally adjacent pair of coil segments arranged along a longitudinal direction of the solenoid, the longitudinally adjacent pair comprising a first coil segment and a second coil segment, each of the first and second coil segments comprising respective windings;
wherein the first and second coil segments are wound on a shared winding-support structure and are arranged so that, at an interface region between the first and second coil segments, windings of the first and second coil segments are permitted to nest, overlap, intermix, or otherwise commingle with one another.

21. The applicator of claim 20, wherein windings of the first and second coil segments collectively form a winding mass along a superior side region of the treatment chamber, the superior side region being configured to face a dorsum of the foot received in the treatment chamber, and wherein, across the interface region, the winding mass is continuous along the superior side region without a winding-free discontinuity between adjacent portions of the winding mass, including without a winding-free discontinuity formed by a stair-step transition between axially adjacent winding stacks.

22. The applicator of claim 20, wherein the interface region includes an actual commingling zone in which windings of the first coil segment and windings of the second coil segment occupy overlapping longitudinal positions, wherein the actual commingling zone has a longitudinal extent of at least 3 mm measured along the longitudinal direction of the solenoid, and wherein, within the actual commingling zone, at least one winding portion of one of the first and second coil segments is positioned at least 3 mm distal of at least one winding portion of the other of the first and second coil segments.

23. The applicator of claim 20, wherein the applicator does not include a first bobbin carrying the first coil segment and a separate second bobbin carrying the second coil segment.

24. The applicator of claim 20, wherein the plurality of separately energizable coil segments comprises at least four coil segments wound on the shared winding-support structure and arranged along the longitudinal direction of the solenoid.

25. The applicator of claim 24, wherein at least three longitudinally adjacent pairs among the at least four coil segments each include a respective actual commingling zone in which windings of both coil segments of the respective longitudinally adjacent pair occupy overlapping longitudinal positions, each respective actual commingling zone having a longitudinal extent of at least 3 mm measured along the longitudinal direction of the solenoid.

26. The applicator of claim 22, wherein, within the actual commingling zone, windings of the first and second coil segments are interleaved through a thickness of a winding mass formed by the windings of the first and second coil segments and measured outward from the treatment chamber, such that a winding portion of one of the first and second coil segments is positioned between winding portions of the other of the first and second coil segments through the thickness of the winding mass.

27. The applicator of claim 21, wherein the shared winding-support structure includes a superior winding-support portion positioned between a superior boundary of the treatment chamber and at least part of the solenoid, wherein the superior winding-support portion carries windings of at least one of the plurality of separately energizable coil segments and extends along a taper-measurement extent of at least 100 mm measured along the distal insertion direction, and wherein, over the taper-measurement extent, a chamber height measured at a longitudinal centerline of the treatment chamber between an inferior boundary of the treatment chamber and the superior boundary of the treatment chamber is at least 30 mm throughout the taper-measurement extent and increases by at least 40 mm from a distal end of the taper-measurement extent to a proximal end of the taper-measurement extent.

28. The applicator of claim 27, wherein, in a longitudinal cross-section through the longitudinal centerline of the treatment chamber, the superior boundary of the treatment chamber defines a curved profile over at least 50 mm of the taper-measurement extent, and wherein a local rate of chamber-height increase along the curved profile does not decrease when moving proximally along the curved profile.
